(19) United States
(12) Patent Application Publication
(10) Pub. No.: US 2002/0198135 A1
(43) Pub. Date: Dec. 26, 2002

(54) Compositions for release of radiosensitizers, and methods of making and using the same

(76) Inventors: Wenbin Dang, Ellicott City, MD (US); Kam W. Leong, Ellicott City, MD (US); Jeffery A. Williams, Baltimore, MD (US)

Correspondence Address: FOLEY HOAG LLP, PATENT GROUP, WORLD TRADE CENTER WEST, 155 SEAPORT BOULEVARD, BOSTON, MA, 02110-2600, US

(21) Appl. No.: 09/976,283

(22) Filed: Oct. 12, 2001

Related U.S. Application Data
(60) Provisional application No. 60/239,807, filed on Oct. 12, 2000.

(51) Int. Cl. A61K 51/00; A61K 31/785

(52) U.S. Cl. 514/1; 424/78.31; 600/1

(57) ABSTRACT

The present invention relates to compositions comprising a biocompatible polymer with phosphorous linkages and a radiosensitizer, and methods of making and using the same.

### RELATED APPLICATION INFORMATION

This application claims the benefit of priority to Provisional Patent Application No. 60/239,807, filed Oct. 12, 2000, which application is hereby incorporated by reference in its entirety.

### INTRODUCTION

A variety of tumors are resistant to standard therapies of surgery, radiation, and systemic chemotherapy. Often the initial therapy is localized surgery to remove as much of the tumor as possible followed by radiation treatments as necessary. Many patients, however, have tumor regrowth within the resected area or contiguous to it (within about 1-3 cm), even after the subsequent radiation treatments.

Radiosensitizers have been used in part to enhance the cytotoxic effect of post-surgical radiation. However, after systemic administration, the clearance of these radiosensitizers from the blood has often been rapid, toxicity has often been high, and the effectiveness has often been low. In addition, the blood-brain barrier clearly limits the delivery of many drugs for the treatment of brain tumors.

Therefore, both the need for treatment and prevention of the recurrence of tumors and the problems encountered with systemic delivery of radiosensitizers have emphasized the need for local delivery of radiosensitizers to predetermined areas of the body. In addition, releasing radiosensitizers directly into the brain tumor area has the possible advantages of circumventing the blood-brain barrier, diminishing systemic exposure to toxic effects, and greatly increasing the therapeutic benefit per unit of drug being administered. Also, intratumoral delivery of radiosensitizer and prolonged delivery of radiosensitizers in a depot form may also prove advantageous.

Sustained release compositions could potentially provide for a sustained, controlled, constant localized release for longer periods of time than can be achieved by other modes of administration. These compositions typically consist of a polymeric matrix or liposome from which drug is released by diffusion and/or degradation of the matrix. The release pattern is usually principally determined by the matrix material, as well as by the percent loading, method of manufacture, type of drug being administered and type of device, for example, microsphere.

In part, the present invention relates to polymer compositions containing a radiosensitizer. In certain embodiments, the polymer comprises a phosphorus linkages in the polymer backbone of a biocompatible and biodegradable polymer, and encapsulation of a radiosensitizer, such a halogenated pyrimidine radiosensitizer, results in release of the radiosensitizer in vivo and in vitro.

### SUMMARY OF THE INVENTION

The present invention is directed in part to polymer compositions having phosphorous-based linkages in which a radiosensitizer is encapsulated. In part, it has been found that such compositions, in combination with electromagnetic radiation, may be used to treat neoplasms and unwanted cell proliferation successfully.

In one aspect, the present invention contemplates biodegradable and biocompatible polymers having phosphorous-based linkages in which a radiosensitizer is encapsulated. In certain embodiments, a large percentage of the subject composition may be a radiosensitizer. For example, the radiosensitizer may comprise 10 to 50% or more of the subject composition, e.g., at least 20%, at least 25%, at least 30%, or more of the composition. Any of the foregoing compositions may include one or more therapeutic agents and other materials in addition to the foregoing radiosensitizer.

In one aspect, the subject polymers may be biocompatible, biodegradable or both. In certain embodiments, the subject polymers contain phosphate, phosphonate and phosphite linkages. In other embodiments, the monomeric units of the present invention have the structures described in the claims appended below, which are hereby incorporated by reference in their entirety into this Summary. In the subject polymers, the chemical structure of certain of the monomeric units may be varied to achieve a variety of desirable physical or chemical characteristics, including for example, release profiles or handling characteristics of the resulting polymer composition.

In certain embodiments, other materials may be encapsulated in the subject polymer in addition to a radiosensitizer to alter the physical and chemical properties of the resulting polymer, including for example, the release profile of the resulting polymer composition for the radiosensitizer. Examples of such materials include biocompatible plasticizers, delivery agents, fillers and the like.

In certain of the foregoing examples, the polymer compositions encapsulating the radiosensitizer remain a flowable gel at room temperature which may be administered by syringe or cannula. In other embodiments, the subject compositions are in the form of microspheres. In still other embodiments, the subject compositions are in the form of nanospheres.

In certain embodiments, administration of the subject polymers results in sustained release of an encapsulated radiosensitizer for a period of time and in an amount that is not possible with other modes of administration. In certain embodiments, any therapeutic agent encapsulated in a subject polymer, e.g., a radiosensitizer, may be controllably released upon administration to a patient. In conjunction with treatment with electromagnetic radiation, such radiosensitizer released in a controlled manner from the subject polymers may be used to treat a variety of diseases and conditions, including neoplastic growths. In certain embodiments, treatment of patient with a subject composition encapsulating a radiosensitizer results in more effective treatment, as measured by a variety of metrics know to those of skill in the art, such as therapeutic index for a treatment regimen, reduction in tumor volume or mass, increased survival rates, increased remission times, than using electromagnetic radiation alone or in combination with the same radiosensitizer administered by other means.

In other embodiments, this invention contemplates a kit including subject compositions, and optionally instructions for their use. Uses for such kits include, for example, therapeutic applications. For example, in one embodiment, such kits include polymer matrices encapsulating a radiosensitizer for use with electromagnetic radiation after administration of such matrices to a patient.

The present invention provides a number of methods of making and using the subject compositions. In part, the subject invention is directed to preparation of the polymeric formulations comprising an radiosensitizer, such as 5-iododeoxyuridine (“IUdR”). Examples of such methods include those disclosed in the claims appended below, which are hereby incorporated by reference in their entirety into this Summary.

In another aspect, the present invention is directed to methods of using the subject polymer compositions for prophylactic or therapeutic treatment. In certain instances, the subject compositions may be used to prevent the recurrence of tumors after surgery to remove the tumor. In certain embodiments, use of the subject compositions, which release in a sustained manner an radiosensitizer allow for different treatment regimens than are possible with other modes of administration of such therapeutic agent.

In another aspect, the subject polymers may be used in the manufacture of a medicament for any number of uses, including for example treating any disease or other treatable condition of a patient. In still other aspects, the present invention is directed to a method for formulating polymers of the present invention in a pharmaceutically acceptable carrier.

The compositions of the present invention, and methods of using the same, have a variety of potentially desirable features, some of which may or may not be present in certain embodiments of the invention. Such features include: (i) the subject compositions may possess sufficient biocompatibility for particular treatments or uses; (ii) a single dose may be sufficient to achieve the desired therapeutically beneficial response through sustained release of the substances incorporated therein; (iii) targeting moieties may be incorporated into the subject compositions for potential targeting of therapeutic agents; (iv) sustained release of an radiosensitizer from a biocompatible, biodegradable polymer composition; (v) novel treatment regimens for treatment of tumors using the subject compositions for sustained delivery of an radiosensitizer; (vi) high levels of loading (by weight), e.g. greater than 10% and up to 50% or more, of an radiosensitizer in the subject polymers; (vi) bioavailability of incorporated materials, including radiosensitizers, may be improved because of protection attributable to subject compositions from serum nuclease degradation and other undesirable reactions that may occur in vivo; (vii) therapeutic agents and other materials may be co-encapsulated in the polymeric formulations; and (vii) the use of the subject polymer matrices may allow targeting on a macro scale of any substance incorporated therein, e.g., physical localization of any radiosensitizer to the brain.

These embodiments of the present invention, other embodiments, and their features and characteristics will be apparent from the description, drawings, and claims that follow.

### BRIEF DESCRIPTION OF THE DRAWINGS

FIG. 1 depicts a SEM showing the morphology of size-reduced IUdR by spray drying.

FIG. 2 depicts a SEM showing the morphology of size-reduced IUdR by precipitation.

FIGS. 3(a) and 3(b) depict SEM and X-Ray data for raw material IUdR.

FIGS. 4(a) and 4(b) depict SEM and X-Ray data for 20% IUdR/P(D,L-APG-EOP) microspheres prepared by spray drying emulsion.

FIG. 5 depicts in vitro release of IUdR from 20% IUdR/P(D,L-APG-EOP) microspheres prepared by spray drying emulsion.

FIGS. 6(a) and 6(b) depict SEM and X-Ray data for 50% IUdR/P(D,L-APG-EOP) microspheres prepared by spray drying emulsion.

FIG. 7 depicts in vitro release of IUdR from 50% IUdR/P(D,L-APG-EOP) microspheres prepared by spray drying emulsion.

FIGS. 8(a) and 8(b) depict SEM and X-Ray data for 15% IUdR/P(D,L-APG-EOP) microspheres prepared by spray drying dispersion.

FIGS. 9(a) and 9(b) depict SEM and X-Ray data for 23% IUdR/P(D,L-APG-EOP) microspheres prepared by solvent dilution method.

FIG. 10 depicts in vitro release of IUdR from 23% IUdR/P(D,L-APG-EOP) microspheres prepared by dilution method.

FIG. 11 depicts the SEM of a 20% IUdR/P(D,L-APG-EOP) rod.

FIG. 12 depicts in vitro release of IUdR from 20% IUdR/P(D,L-APG-EOP) rod.

FIG. 13 depicts a 50% IUdR/P(D,L-APG-EOP) rod.

FIG. 14 depicts in vitro release of IUdR from 50% IUdR/P(D,L-APG-EOP) rod.

FIGS. 15(a) and 15(b) depict SEM and X-Ray data for 20% IUdR/P(D,L-APG-EOP) microparticles.

FIG. 16 depicts in vitro release of IUdR from 20% IUdR/P(D,L-APG-EOP) microparticles.

FIGS. 17(a) and 17(b) depict SEM and X-Ray data for 50% IUdR/P(D,L-APG-EOP) microparticles.

FIG. 18 depicts in vitro release of IUdR from 50% IUdR/P(D,L-APG-EOP) microparticles.

FIG. 19 depicts in vitro release of IUdR from 25% IUdR/P(trans-CHDM/HOP) paste.

FIGS. 20(a) and 20(b) depict SEM and X-Ray data for 20% IUdR/P(BHET-EOP/TC) film.

FIG. 21 depicts in vitro release of IUdR from 20% IUdR/P(BHET-EOP/TC) film.

FIGS. 22(a) and 22(b) depict SEM and X-Ray data for 50% IUdR/P(BHET-EOP/TC) film.

FIG. 23 depicts in vitro release of IUdR from 50% IUdR/P(BHET-EOP/TC) film.

FIG. 24. Volume of xenograft in flank of mice receiving external beam irradiation in conjunction with 16.7% (by weight) IUdR/P(trans-CHDM/HOP) paste.

FIG. 25. Volume of xenograft in flank of mice receiving ULDR and HDR (both as defined below) in conjunction with 16.7% (by weight) IUdR/P(trans-CHDM/HOP) paste, as described in Example 29.

FIGS. 26 and 27 show the results of Example 28 using D,L-PL(PG)EOP loaded with IUdR, with FIG. 26 showing microspheres and FIG. 27 showing rods of the subject compositions.

### DETAILED DESCRIPTION OF THE INVENTION

1. Overview

The present invention relates to pharmaceutical compositions for the delivery of radiosensitizers, such as IUdR, for the treatment of neoplasms upon radiation (both alone and in conjunction with other treatment regiments, such as surgery). In certain embodiments, biodegradable, biocompatible polymers may be used to allow for sustained release of an encapsulated radiosensitizer. The present invention also relates to methods of administering such pharmaceutical compositions, e.g., as part of a treatment regimen.

In certain aspects, the subject pharmaceutical compositions, upon contact with body fluids including blood, spinal fluid, lymph or the like, release the encapsulated radiosensitizer over a sustained or extended period (as compared to the release from an isotonic saline solution). Such a system may result in prolonged delivery (over, for example, 8 to 800 hours, preferably 24 to 480 or more hours) of effective amounts (e.g., 0.0001 mg/kg/hour to 10 mg/kg/hour) of the drug. This dosage form may be administered as is necessary depending on the subject being treated, the severity of the affliction, the judgment of the prescribing physician, and the like.

2. Definitions

For convenience, before further description of the present invention, certain terms employed in the specification, examples, and appended claims are collected here. These definitions should be read in light of the remainder of the disclosure and understood as by a person of skill in the art. Also, the terms “including” (and variants thereof), “such as”, “e.g.” as used in this specification are non-limiting and are for illustrative purposes only.

“Radiosensitizer” is an art-recognized term and is defined as a therapeutic agent that, upon administration in a therapeutically effective amount, promotes the treatment of one or more diseases or conditions that are treatable with electromagnetic radiation. Diseases that are treatable with electromagnetic radiation include, for example, neoplastic diseases, benign and malignant tumors, cancerous cells, restenosis, atherosclerotic plaque, neovascular lesions, e.g., moles and birthmarks, and the like. In general, radiosensitizers are intended to be used in conjunction with electromagnetic radiation as part of a prophylactic or therapeutic treatment. Electromagnetic radiation treatment of other diseases so treatable, but which are not listed herein, are also contemplated by the present invention. Examples of radiosensitizers are set forth below in the next section. In certain embodiments, the radiosensitizer used is a halogenated pyrimidine such as BUdR, IUdR, FUdR, IPdR and the like, and in still other embodiments, the radiosensitizer used is IUdR. A subclass of radiosensitizers are those radiosensitizers which do not have an acceptable therapeutic index for treatment unless used in conjunction with electromagnetic radiation, which are hereafter referred to as “type I radiosensitizers”.

“Electromagnetic radiation” as used in this specification includes, but is not limited to, radiation having the wavelength of 10⁻²⁰ to 10 meters. Particular embodiments of electromagnetic radiation of the present invention employ the electromagnetic radiation of: gamma-radiation (10⁻²⁰ to 10⁻¹³ m), x-ray radiation (10⁻¹¹ to 10⁻⁹ m), ultraviolet light (10 nm to 400 nm), visible light (400 nm to 700 nm), infrared radiation (700 nm to 1.0 mm), and microwave radiation (1 mm to 30 cm).

The term “access device” is an art-recognized term and includes any medical device adapted for gaining or maintaining access to an anatomic area. Such devices are familiar to artisans in the medical and surgical fields. An access device may be a needle, a catheter, a cannula, a trocar, a tubing, a shunt, a drain, or an endoscope such as a laparoscope, cystoscope, sigmoidoscope, or any other endoscope adapted for use in an anatomic area affected by a prostate cancer, or any other medical device suitable for entering or remaining positioned within the preselected anatomic area.

The terms “biocompatible polymer” and “biocompatibility” when used in relation to polymers are art-recognized. For example, biocompatible polymers include polymers that are neither themselves toxic to the host (e.g., an animal or human), nor degrade (if the polymer degrades) at a rate that produces monomeric or oligomeric subunits or other byproducts at toxic concentrations in the host. In certain embodiments of the present invention, biodegradation generally involves degradation of the polymer in an organism, e.g., into its monomeric subunits, which may be known to be effectively non-toxic. Intermediate oligomeric products resulting from such degradation may have different toxicological properties, however, or biodegradation may involve oxidation or other biochemical reactions that generate molecules other than monomeric subunits of the polymer. Consequently, in certain embodiments, toxicology of a biodegradable polymer intended for in vivo use, such as implantation or injection into a patient, may be determined after one or more toxicity analyses. It is not necessary that any subject composition have a purity of 100% to be deemed biocompatible; indeed, it is only necessary that the subject compositions be biocompatible as set forth above. Hence, a subject composition may comprise polymers comprising 99%, 98%, 97%, 96%, 95%, 90%, 85%, 80%, 75% or even less of biocompatible polymers, e.g., including polymers and other materials and excipients described herein, and still be biocompatible.

To determine whether a polymer or other material is biocompatible, it may be necessary to conduct a toxicity analysis. Such assays are well known in the art. One example of such an assay may be performed with live carcinoma cells, such as GT3TKB tumor cells, in the following manner: the sample is degraded in 1M NaOH at 37° C. until complete degradation is observed. The solution is then neutralized with 1M HCl. About 200 μL of various concentrations of the degraded sample products are placed in 96-well tissue culture plates and seeded with human gastric carcinoma cells (GT3TKB) at 10⁴/well density. The degraded sample products are incubated with the GT3TKB cells for 48 hours. The results of the assay may be plotted as % relative growth vs. concentration of degraded sample in the tissue-culture well. In addition, polymers and formulations of the present invention may also be evaluated by well-known in vivo tests, such as subcutaneous implantations in rats to confirm that they do not cause significant levels of irritation or inflammation at the subcutaneous implantation sites.

The term “biodegradable” is art-recognized, and includes polymers, compositions and formulations, such as those described herein, that are intended to degrade during use. Biodegradable polymers typically differ from non-biodegradable polymers in that the former may be degraded during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to side chain or that connects a side chain to the polymer backbone. For example, a therapeutic agent or other chemical moiety attached as a side chain to the polymer backbone may be released by biodegradation. In certain embodiments, one or the other or both generally types of biodegradation may occur during use of a polymer. As used herein, the term “biodegradation” encompasses both general types of biodegradation.

The degradation rate of a biodegradable polymer often depends in part on a variety of factors, including the chemical identity of the linkage responsible for any degradation, the molecular weight, crystallinity, biostability, and degree of cross-linking of such polymer, the physical characteristics of the implant, shape and size, and the mode and location of administration. For example, the greater the molecular weight, the higher the degree of crystallinity, and/or the greater the biostability, the biodegradation of any biodegradable polymer is usually slower. The term “biodegradable” is intended to cover materials and processes also termed “bioerodible”.

In certain embodiments, if the biodegradable polymer also has a therapeutic agent or other material associated with it, the biodegradation rate of such polymer may be characterized by a release rate of such materials. In such circumstances, the biodegradation rate may depend on not only the chemical identity and physical characteristics of the polymer, but also on the identity of any such material incorporated therein.

In certain embodiments, polymeric formulations of the present invention biodegrade within a period that is acceptable in the desired application. In certain embodiments, such as in vivo therapy, such degradation occurs in a period usually less than about five years, one year, six months, three months, one month, fifteen days, five days, three days, or even one day on exposure to a physiological solution with a pH between 6 and 8 having a temperature of between 25 and 37° C. In other embodiments, the polymer degrades in a period of between about one hour and several weeks, depending on the desired application.

When used with respect to a radiosensitizer or other material, the term “sustained release” is art-recognized. For example, a subject composition which releases a substance over time may exhibit sustained release characteristics, in contrast to a bolus type administration in which the entire amount of the substance is made biologically available at one time. For example, in particular embodiments, upon contact with body fluids including blood, spinal fluid, lymph or the like, the polymer matrices (formulated as provided herein and otherwise as known to one of skill in the art) may undergo gradual degradation (e.g., through hydrolysis) with concomitant release of any material incorporated therein, e.g., a radiosensitizer such as IUdR, for a sustained or extended period (as compared to the release from a bolus). This release may result in prolonged delivery of therapeutically effective amounts of any incorporated therapeutic agent. Sustained release will vary in certain embodiments as described in greater detail below.

The term “delivery agent” is an art-recognized term, and includes molecules that facilitate the intracellular delivery of a therapeutic agent or other material. Examples of delivery agents include: sterols (e.g., cholesterol) and lipids (e.g., a cationic lipid, virosome or liposome).

The term “drug delivery device” is an art-recognized term and refers to any medical device suitable for the application of a drug or therapeutic agent to the targeted organ or anatomic region. The term includes, without limitation, those formulations of the compositions of the present invention that release the radiosensitizer into the surrounding tissues. The term further includes those devices that transport or accomplish the instillation of the compositions of the present invention towards the targeted organ or anatomic region, even if the device itself is not formulated to include the composition. As an example, a needle or a catheter through which the composition is inserted into the body cavity is understood to be a drug delivery device. As a further example, a stent or a shunt or a catheter that has the composition included in its substance or coated on its surface is understood to be a drug delivery device.

The term “microspheres” is art-recognized, and includes substantially spherical colloidal structures, e.g., formed from biocompatible polymers such as subject compositions, having a size ranging from about one or greater up to about 1000 microns. In general, “microcapsules”, also an art-recognized term, may be distinguished from microspheres, because microcapsules are generally covered by a substance of some type, such as a polymeric formulation. The term “microparticles” is art-recognized, and includes microspheres and microcapsules, as well as structures that may not be readily placed into either of the above two categories, all with deimensions on avreage of less than 1000 microns. If the structures are less than about one micron in diameter, then the corresponding art-recognized terms “nanosphere,” “nanocapsule,” and “nanoparticle” may be utilized. In certain embodiments, the nanospheres, nancapsules and nanoparticles have an average diameter of about 500, 200, 100, 50 or 10 nm.

A composition comprising microspheres may include particles of a range of particle sizes. In certain embodiments, the particle size distribution may be uniform, e.g., within less than about a 20% standard deviation of the median volume diameter, and in other embodiments, still more uniform or within about 10% of the median volume diameter.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrastemal injection and infusion.

The term “treating” is an art-recognized term which includes curing as well as ameliorating at least one symptom of any condition or disease. Treating includes preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder or condition, e.g., causing regression of the disease, disorder and/or condition. Further, treating the disease or condition includes ameliorating at least one symptom of the particular disease or condition, even if the underlying pathophysiology is not affected.

The term “fluid” is art-recognized to refer to a non-solid state of matter in which the atoms or molecules are free to move in relation to each other, as in a gas or liquid. If unconstrained upon application, a fluid material may flow to assume the shape of the space available to it, covering for example, the cavity created by excision of a tumor. A fluid material may be inserted or injected into a limited portion of a space and then may flow to enter a larger portion of the space or its entirety. Such a material may be termed “flowable.” This term is art-recognized and includes, for example, liquid compositions that are capable of being sprayed into a site; injected with a manually operated syringe fitted with, for example, a 23-gauge needle; or delivered through a catheter. Also included in the term “flowable” are those highly viscous, “gel-like” materials at room temperature that may be delivered to the desired site by pouring, squeezing from a tube, or being injected with any one of the commercially available injection devices that provide injection pressures sufficient to propel highly viscous materials through a delivery system such as a needle or a catheter. When the polymer used is itself flowable, a composition comprising it need not include a biocompatible solvent to allow its dispersion within a body cavity. Rather, the flowable polymer may be delivered into the body cavity using a delivery system that relies upon the native flowability of the material for its application to the desired tissue surfaces. For example, if flowable, a composition comprising polymers according to the present invention it can be injected to form, after injection, a temporary biomechanical barrier to coat or encapsulate internal organs or tissues, or it can be used to produce coatings for solid implantable devices. In certain instances, flowable subject compositions has the ability to assume, over time, the shape of the space containing it at body temperature.

Viscosity is understood herein as it is recognized in the art to be the internal friction of a fluid or the resistance to flow exhibited by a fluid material when subjected to deformation. The degree of viscosity of the polymer can be adjusted by the molecular weight of the polymer, as well as by mixing different isomers of the polymer backbone; other methods for altering the physical characteristics of a specific polymer will be evident to practitioners of ordinary skill with no more than routine experimentation. The molecular weight of the polymer used in the composition of the invention can vary widely, depending on whether a rigid solid state (usually higher molecular weights) desirable, or whether a fluid state (usually lower molecular weights) is desired.

The phrase “pharmaceutically acceptable” is art-recognized. In certain embodiments, the term includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, excipient, solvent or encapsulating material, involved in carrying or transporting any subject composition, from one organ, or portion of the body, to another organ, or portion of the body. Each carrier must be “acceptable” in the sense of being compatible with the other ingredients of the composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically acceptable salts” is art-recognized, and includes relatively non-toxic, inorganic and organic acid addition salts of compositions of the present invention, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

A “patient,” “subject,” or “host” to be treated by the subject method may mean either a human or non-human animal, such as primates, mammals, and vertebrates.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The term “preventing,” when used in relation to a condition, such as a local recurrence, a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

The phrases “systemic administration,” “administered systemically,” “peripheral administration” and “administered peripherally” are art-recognized, and include the administration of a subject composition or other material at a site remote from the disease being treated. Administration of an agent directly into, onto or in the vicinity of a lesion of the disease being treated, even if the agent is subsequently distributed systemically, may be termed “local” or “topical” or “regional” administration, other than directly into the central nervous system, e.g., by subcutaneous administration, such that it enters the patient's system and, thus, is subject to metabolism and other like processes.

The phrase “therapeutically effective amount” is an art-recognized term. In certain embodiments, the term refers to an amount of the therapeutic agent that, when incorporated into a polymer of the present invention, produces some desired effect at a reasonable benefit/risk ratio applicable to any medical treatment. In certain embodiments, the term refers to that amount necessary or sufficient to eliminate, reduce or maintain (e.g., prevent the spread of) a tumor or other target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation.

In certain embodiments, a therapeutically effective amount of a radiosensitizer, such as IUdR, for in vivo use will likely depend on a number of factors, including: the rate of release of the agent from the polymer matrix, which will depend in part on the chemical and physical characteristics of the polymer; the identity of the agent; the mode and method of administration; the treatment regimen of electromagnetic radiation to be used in conjunction with the polymer composition; and any other materials incorporated in the polymer matrix in addition to the radiosensitizer.

The term “ED₅₀” is art-recognized. In certain embodiments, ED₅₀ means the dose of a drug which produces 50% of its maximum response or effect, or alternatively, the dose which produces a pre-determined response in 50% of test subjects or preparations. The term “LD₅₀” is art-recognized. In certain embodiments, LD₅₀ means the dose of a drug which is lethal in 50% of test subjects. The term “therapeutic index” is an art-recognized term which refers to the therapeutic index of a drug, defined as LD₅₀/ED₅₀. The same terminology may be used in referring to treatment with electromagnetic radiation. For example, in certain embodiments, ED₅₀ means the amount of radiation that results in a 50% growth inhibition of a neoplasm or other tumor.

The terms “incorporated” and “encapsulated” are art-recognized when used in reference to a therapeutic agent, or other material and a polymeric composition, such as a composition of the present invention. In certain embodiments, these terms include incorporating, formulating or otherwise including such agent into a composition which allows for sustained release of such agent in the desired application. The terms may contemplate any manner by which a therapeutic agent or other material is incorporated into a polymer matrix, including for example: attached to a monomer of such polymer (by covalent or other binding interaction) and having such monomer be part of the polymerization to give a polymeric formulation, distributed throughout the polymeric matrix, appended to the surface of the polymeric matrix (by covalent or other binding interactions), encapsulated inside the polymeric matrix, etc. The term “co-incorporation” or “co-encapsulation” refers to the incorporation of a therapeutic agent or other material and at least one other therapeutic agent or other material in a subject composition.

More specifically, the physical form in which any therapeutic agent or other material is encapsulated in polymers may vary with the particular embodiment. For example, a therapeutic agent or other material may be first encapsulated in a microsphere and then combined with the polymer in such a way that at least a portion of the microsphere structure is maintained. Alternatively, a therapeutic agent or other material may be sufficiently immiscible in the polymer of the invention that it is dispersed as small droplets, rather than being dissolved, in the polymer. Any form of encapsulation or incorporation is contemplated by the present invention, in so much as the sustained release of any encapsulated therapeutic agent or other material determines whether the form of encapsulation is sufficiently acceptable for any particular use.

The term “biocompatible plasticizer” is art-recognized, and includes materials which are soluble or dispersible in the compositions of the present invention, which increase the flexibility of the polymer matrix, and which, in the amounts employed, are biocompatible. Suitable plasticizers are well known in the art and include those disclosed in U.S. Pat. Nos. 2,784,127 and 4,444,933. Specific plasticizers include, by way of example, acetyl tri-n-butyl citrate (c. 20 weight percent or less), acetyl trihexyl citrate (c. 20 weight percent or less), butyl benzyl phthalate, dibutyl phthalate, dioctylphthalate, n-butyryl tri-n-hexyl citrate, diethylene glycol dibenzoate (c. 20 weight percent or less) and the like.

“Small molecule” is an art-recognized term. In certain embodiments, this term refers to a molecule which has a molecular weight of less than about 2000 amu, or less than about 1000 amu, and even less than about 500 amu.

The term “aliphatic” is an art-recognized term and includes linear, branched, and cyclic alkanes, alkenes, or alkynes. In certain embodiments, aliphatic groups in the present invention are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Moreover, the term “alkyl” (or “lower alkyl”) includes both “unsubstituted alkyls” and “substituted alkyls”, the latter of which refers to alkyl moieties having substituents replacing a hydrogen on one or more carbons of the hydrocarbon backbone. Such substituents may include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls may be further substituted with alkyls, alkenyls, alkoxys, alkylthios, aminoalkyls, carbonyl-substituted alkyls, —CF₃, —CN, and the like.

The term “aralkyl” is art-recognized, and includes alkyl groups substituted with an aryl group (e.g., an aromatic or heteroaromatic group).

The terms “alkenyl” and “alkynyl” are art-recognized, and include unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to ten carbons, alternatively from one to about six carbon atoms in its backbone structure. Likewise, “lower alkenyl” and “lower alkynyl” have similar chain lengths.

The term “heteroatom” is art-recognized, and includes an atom of any element other than carbon or hydrogen. Illustrative heteroatoms include boron, nitrogen, oxygen, phosphorus, sulfur and selenium, and alternatively oxygen, nitrogen or sulfur.

The term “aryl” is art-recognized, and includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and apply to 1,2-, 1,3- and 1,4-disubstituted benzenes, respectively. For example, the names 1,2-dimethylbenzene and ortho-dimethylbenzene are synonymous.

The terms “heterocyclyl” and “heterocyclic group” are art-recognized, and include 3- to about 10-membered ring structures, such as 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” and “polycyclic group” are art-recognized, and include structures with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms, e.g., three or more atoms are common to both rings, are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art recognized and includes an aromatic or non-aromatic ring in which each atom of the ring is carbon. The flowing art-recognized terms have the following meanings: “nitro” means —NO₂; the term “halogen” designates —F, —Cl, —Br or —I; the term “sulfhydryl” means —SH; the term “hydroxyl” means —OH; and the term “sulfonyl” means —SO₂⁻.

The terms “amine” and “amino” are art-recognized and include both unsubstituted and substituted amines, e.g., a moiety that may be represented by the general formulas:

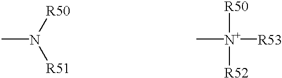

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and includes a moiety that may be represented by the general formula:

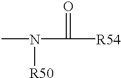

wherein R50 is as defined above, and R54 represents a hydrogen, an alkyl, an alkenyl or —(CH2)ₘ—R61, where m and R61 are as defined above.

The term “amido” is art-recognized as an amino-substituted carbonyl and includes a moiety that may be represented by the general formula:

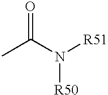

wherein R50 and R51 are as defined above. Certain embodiments of the amide in the present invention will not include imides which may be unstable.

The term “alkylthio” is art-recognized and includes an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” is art-recognized and includes such moieties as may be represented by the general formulas:

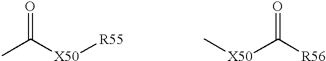

wherein X50 is a bond or represents an oxygen or a sulfur, and R55 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiocarbonyl” group. Where X50 is a sulfur and R55 or R56 is not hydrogen, the formula represents a “thioester.” Where X50 is a sulfur and R55 is hydrogen, the formula represents a “thiocarboxylic acid.” Where X50 is a sulfur and R56 is hydrogen, the formula represents a “thioformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art-recognized and include an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O-(CH₂)ₘ—R61, where m and R61 are described above.

The term “sulfonate” is art-recognized and includes a moiety that may be represented by the general formula:

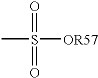

in which R57 is an electron pair, hydrogen, alkyl, cycloalkyl, or aryl.

The term “sulfate” is art-recognized and includes a moiety that may be represented by the general formula:

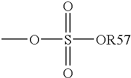

in which R57 is as defined above.

The term “sulfonamido” is art-recognized and includes a moiety that may be represented by the general formula:

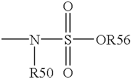

in which R50 and R56 are as defined above.

The term “sulfamoyl” is art-recognized and includes a moiety that may be represented by the general formula:

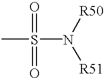

in which R50 and R51 are as defined above.

The term “sulfonyl” is art-recognized and includes a moiety that may be represented by the general formula:

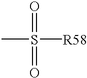

in which R58 is one of the following: hydrogen, alkyl, alkenyl, alkynyl, cycloalkyl, heterocyclyl, aryl or heteroaryl.

The term “sulfoxido” is art-recognized and includes a moiety that may be represented by the general formula:

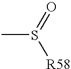

in which R58 is defined above.

The term “phosphoramidite” is art-recognized and includes moieties represented by the general formulas:

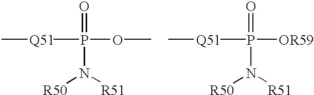

wherein Q51, R50, R51 and R59 are as defined above.

The term “phosphonamidite” is art-recognized and includes moieties represented by the general formulas:

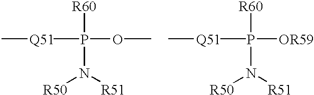

wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, etc., when it occurs more than once in any structure, is intended to be independent of its definition elsewhere in the same structure unless otherwise indicated expressly or by the context.

The term “selenoalkyl” is art-recognized and includes an alkyl group having a substituted seleno group attached thereto. Exemplary “selenoethers” which may be substituted on the alkyl are selected from one of —Se-alkyl, —Se-alkenyl, —Se-alkynyl, and —Se-(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms are art-recognized and represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain monomeric subunits of the present invention may exist in particular geometric or stereoisomeric forms. In addition, polymers and other compositions of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

For purposes of this invention, the chemical elements are identified in accordance with the Periodic Table of the Elements, CAS version, Handbook of Chemistry and Physics, 67th Ed., 1986-87, inside cover. The term “hydrocarbon” is art recognized and includes all permissible compounds having at least one hydrogen and one carbon atom. For example, permissible hydrocarbons include acyclic and cyclic, branched and unbranched, carbocyclic and heterocyclic, aromatic and nonaromatic organic compounds that may be substituted or unsubstituted.

The phrase “protecting group” is art-recognized and includes temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed. Greene et al., Protective Groups in Organic Synthesis 2^{nd} ed., Wiley, New York, (1991).

The phrase “hydroxyl-protecting group” is art-recognized and includes those groups intended to protect a hydroxyl group against undesirable reactions during synthetic procedures and includes, for example, benzyl or other suitable esters or ethers groups known in the art.

The term “electron-withdrawing group” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, March, Advanced Organic Chemistry 251-59, McGraw Hill Book Company, New York, (1977). The Hammett constant values are generally negative for electron donating groups (σ(P)=−0.66 for NH₂) and positive for electron withdrawing groups (σ(P)=0.78 for a nitro group), σ(P) indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

Contemplated equivalents of the polymers, subunits and other compositions described above include such materials which otherwise correspond thereto, and which have the same general properties thereof (e.g., biocompatible, radiosensitizers), wherein one or more simple variations of substituents are made which do not adversely affect the efficacy of such molecule to achieve its intended purpose. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

3. Exemplary Subject Compositions, and Methods of Making and Using the Same

A. Radiosensitizers

Generally, radiosensitizers are known to increase the sensitivity of cancerous cells or other unwanted cells or tissue to the effects of electromagnetic radiation. This section presents examples of such radiosensitizers and some of their possible uses, categorized in a general fashion by the type of electromagnetic radiation with which each particular type of radiosensitizer is often employed. These examples are not intended to limit the potential uses of the specified radiosensitizers, including with what type of electromagnetic radiation any of them may be used as part of a prophylactic or therapeutic treatment. Also, radiosensitizers are one category of therapeutic agents, which are addressed in greater detail below.

While not wishing to be bound by any particular theory, and without limiting any embodiment of the invention to a particular mechanism, several mechanisms for the mode of action of radiosensitizers have been proposed in the literature. For example, hypoxic cell radiosensitizers, such as 2-nitroimidazole compounds and benzotriazine dioxide compounds, are believed to promote the re-oxygenation of hypoxic tissue and/or catalyze the generation of damaging oxygen radicals. Hypoxic, i.e., oxygen deficient, cells are relatively resistant to killing by radiation. To achieve the same proportion of cell kill, about three times the radiation dose is required for hypoxic cells as compared to the radiation dose required for well-oxygenated cells. Overcoming this resistance of hypoxic cells has been investigated as a means of improving the efficacy of ionizing radiation, and have involved the development of hypoxic cell radiosensitizers.

Multiple mechanisms have been proposed to explain hypoxic resistance to radiation therapy and chemotherapy, and these proposed mechanisms involve kinetic, metabolic and physical factors. For example, hypoxic cells frequently are noncycling and therefore are refractory to proliferation-dependent cytotoxic drugs. In addition, the cell may be in a metabolically compromised state and unable to concentrate and activate potentially-effective agents. The distance between a cell and blood vessels may also be greater than the diffusion distance of many chemotherapeutic agents. Other mechanisms may also be involved.

In contrast, non-hypoxic cell radiosensitizers, such as halogenated pyrimidines, are capable of being preferentially incorporated into the DNA of cancer cells thereby promoting the radiation-induced disruption of DNA molecules and/or preventing the normal DNA repair mechanisms. Various other potential mechanisms of action have also been hypothesized for radiosensitizers in the treatment of different diseases.

Radiosensitizers that are activated by the electromagnetic radiation of x-rays are currently used in many cancer treatment protocols. Examples of radiosensitizers activated by x-rays include the following: metronidazole, misonidazole, desmethylmisonidazole, pimonidazole, etanidazole, nimorazole, mitomycin C, RSU 1069, SR 4233, E09, RB 6145, nicotinamide, 5-bromodeoxyuridine (BUdR), BUdR/Broxine (made by Neopharm), 5-iododeoxyuridine (IUdR), bromodeoxycytidine, fluorodeoxyuridine (FUdR), hydroxyurea, cisplatin and analogs, and derivatives of these compounds. The foregoing examples include therapeutic agents that are either hypoxic and non-hypoxic cell radiosensitizers (or both).

Photodynamic therapy (PDT) uses visible light as the electromagnetic radiation. Examples of radiosensitizers used with PDT include the following: hematoporphyrin derivatives, benzoporphyrin derivatives, NPe6, tin etioporphyrin SnET2, pheophorbide-a, bacteriochlorophyll-a, naphthalocyanines, phthalocyanines (such as zinc phthalocyanine), and analogs and derivatives of the above. In general, PDT procedures function selectively to eradicate diseased tissue in the immediate area of the light source by generating singlet oxygen and activated molecules which damage tissue in that immediate area. Selectivity is believed to be attained through the preferential retention of the photosensitizer in rapidly metabolizing tissue such as tumors.

Additional examples of particular radiosensitizers include metoclopramide, sensamide or neusensamide (manufactured by Oxigene); profiromycin (made by Vion); RSR13 (made by Allos); Thymitaq (made by Agouron); lobenguane (manufactured by Nycomed); gadolinium texaphrin (made by Pharmacyclics); IPdR (made by Sparta); CR2412 (made by Cell Therapeutic); L1X (made by Terrapin); and the like.

Other examples of radiosensitizers include those chemical moieties that inhibit PARP (as defined below), or “PARP inhibitors”. It is believed that inhibition of the formation of poly(ADP-ribose) impairs the cellular recovery from DNA damage associated with electromagnetic radiation, and in particular, gamma irradiation. Poly(ADP-ribosyl)ation is a post-translational modification of nuclear proteins catalyzed by poly(ADP-ribose)polymerase (“PARP”), an enzyme that uses NAD+ as substrate. The binding of PARP to DNA single-strand or double-strand breaks leads to enzyme activation.

Examples of useful PARP radiosensitizers include categories of compounds that inhibit PARP, such as benzamides, benzamide derivatives, phenanthridones, isoquinolines, dihydroisoquinolines, dihydroxyisoquinolines, isoquinolinones, quinazolines, quinazolinones, naphthalimides and hydroxybenzamides. When, the radiosensitizer is a PARP inhibitor, it is occasionally selected from the group consisting of isoquinolines, dihydroisoquinolines, dihydroxyisoquinolines, isoquinazolinones, naphthalamides. Alternatively, when the radiosensitizer is a PARP inhibitor, it is an isoquinolinone.

Others examples of useful PARP inhibitors include: benzoic acid, 3-aminobenzamide, 4-aminobenzamide, 3-acetamidobenzamide, 3-chlorobenzamide, 3-hydroxybenzamide, 3-methylbenzamide, 3-methoxybenzamide, benzoyleneurea, 6-amino-1,2-benzopyrone, trp-P-1(3-amino-1,4-dimethyl-5H-pyrido[4,3-b]indole), 1-hydroxyisoquinoline, 1,5-dihydroxyisoquinoline, 3,4-dihydro-5-[4-(1 -piperidinyl)-butox]-1 (2H)-isoquinolinone, juglone (a natural quinone), luminol, 1,8-naphthalimide, 4-amino-1,8-naphthalimide, N-hydroxynaphthalimide sodium salt, 1(2H)-phthalazinone, phthalhydrazide, 6(5H)-phenanthridinone, 2-nitro-6(5H)-phenanthridinone, 4-hydroxyquinazoline, 2-methyl-4(3H)-quinozoline, 2-methyl-4(3H)-quinazolinone, 2-mercapto-4(3H)-quinazolinone and chlorthenoxazin.

Many of the radiosensitizers are known and, thus, may be synthesized by known methods from starting materials that are known, may be available commercially, or may be prepared by methods used to prepare corresponding compounds in the literature.

B. Polymers

A variety of polymers having phosphate linkages may be used in the subject invention. Both non-biodegradable and biodegradable polymers may be used in the subject invention, although biodegradable polymers are preferred. As discussed below, the choice of polymer will depend in part on a variety of physical and chemical characteristics of such polymer and the use to which such polymer may be put.

Exemplary phosphorus linkages in such polymers include, without limitation, phosphonamidite, phosphoramidite, phosphorodiamidate, phosphomonoester, phosphodiester, phosphotriester, phosphonate, phosphonate ester, phosphorothioate, thiophosphate ester, phosphinate or phosphite. Any of the subject polymers may be provided as copolymers, terpolymers, etc. Certain of such polymers may be biodegradable, biocompatible or both.

The structure of certain of the foregoing polymers having phosphorus linkages may be identified as follows. The term “polymer having phosphorous-based linkages” is used herein to refer to polymers in which the following substructure is present at least a multiplicity of times in the backbone of such polymer:

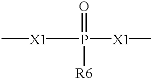

wherein, independently for each occurrence of such substructure:

X1, each independently, represents —O— or —N(R5)-;

R5 represents —H, aryl, alkenyl or alkyl; and

R6 is any non-interfering substituent,

wherein such substructure is responsible in part for biodegradability properties, if any, observed for such polymer in vitro or in vivo. In certain embodiments, R6 may represent an alkyl, aralkyl, alkoxy, alkylthio, or alkylamino group.

In certain embodiments, such a biodegradable polymer is non-naturally occurring, i.e., a man-made product with no natural source. In other embodiments, R6 is not —OH or halogen, e.g., is an alkyl, aralkyl, aryl, alkoxyl, aryloxy, or aralkyloxy. In still other embodiments, the two X1 moieties in such substructure are the same. For general guidance, when reference is made to the “polymer backbone chain” or the like of a polymer, with reference to the above structure, such polymer backbone chain comprises the motif [—X1-P—X1-]. In other polymers, the polymer backbone chain may vary as recognized by one of skill in the art.

By way of example, but not limitation, a number of representative polymers having phosphorus linkages are described in greater detail below. In certain embodiments, a polymer includes one or more monomeric units of Formula V:

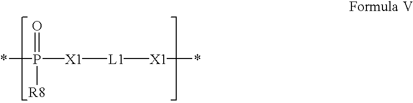

wherein, independently for each occurrence of such unit:

X1, each independently, represents —O— or —N(R7)-;

R7 represents —H, aryl, alkenyl or alkyl;

L1 is described below;

R8 represents, for example, —H, alkyl, —O-alkyl, —O-cycloalkyl, aryl, —O-aryl, heterocycle, —O-heterocycle, —N(R9)R10 and other examples presented below;

R9 and R10, each independently, represent a hydrogen, an alkyl, an alkenyl, —(CH2)ₘ—R11, or R9 and R10, taken together with the N atom to which they are attached complete a heterocycle having from 4 to about 8 atoms in the ring structure;

m represents an integer in the range of 0-10, preferably 0-6; and

R1 represents —H, alkyl, aryl, cycloalkyl, cycloalkenyl, heterocycle or polycycle.

L1 may be any chemical moiety as long as it does not materially interfere with the polymerization, biocompatibility or biodegradation (or any combination of those three properties) of the polymer, wherein a “material interference” or “non-interfering substituent” is understood to mean: (i) for synthesis of the polymer by polymerization, an inability to prepare the subject polymer by methods known in the art or taught herein; (ii) for biocompatability, a reduction in the biocompatability of the subject polymer so as to make such a polymer impraticable for in vivo use; and (iii) for biodegradation, a reduction in the biodegradation of the subject polymer so as to make such polymer impracticable for biodegradation.

In certain embodiments, L1 is an organic moiety, such as a divalent branched or straight chain or cyclic aliphatic group or divalent aryl group, with in certain embodiments, from 1 to about 20 carbon atoms. In certain embodiments, L1 represents a moiety between about 2 and 20 atoms selected from carbon, oxygen, sulfur, and nitrogen, wherein at least 60% of the atoms are carbon. In certain embodiments, L1 may be an alkylene group, such as methylene, ethylene, 1,2-dimethylethylene, n-propylene, isopropylene, 2,2-dimethylpropylene, n-pentylene, n-hexylene, n-heptylene; an alkenylene group such as ethenylene, propenylene, 2-(3-propenyl)-dodecylene; and an alkynylene group such as ethynylene, proynylene, 1-(4-butynyl)-3-methyldecylene; and the like. Such unsaturated aliphatic groups may be used to cross-link certain embodiments of the present invention.

Further, L1 may be a cycloaliphatic group, such as cyclopentylene, 2-methylcyclopentylene, cyclohexylene, cyclohexylenedimethylene, cyclohexenylene and the like. L1 may also be a divalent aryl group, such as phenylene, benzylene, naphthalene, phenanthrenylene and the like. Further, L1 may be a divalent heterocyclic group, such as pyrrolylene, furanylene, thiophenylene, alkylyene-pyrrolylene-alkylene, pyridinylene, pyrimidinylene and the like.

Other examples of L1 may include any of the polymers listed above, including the biodegradable polymers listed above, and in particular polylactide, polyglycolide, polycaprolactone, polycarbonate, polyethylene terephthalate, polyanhydride and polyorthoester, and polymers of ethylene glycol, propylene glycol and the like. Embodiments containing such polymers for L1 may impart a variety of desired physical and chemical properties.

The foregoing, as with other moieties described herein, may be substituted with a non-interfering substituent, for example, a hydroxy-, halogen-, or nitrogen-substituted moiety.

R8 represents hydrogen, alkyl, cycloakyl, —O-alkyl, —O-cycloalkyl, aryl, —O-aryl, heterocycle, —O-heterocycle, or —N(R9)R10. Examples of possible alkyl R8 groups include methyl, ethyl, n-propyl, i-propyl, n-butyl, tert-butyl, —C₈H₁₇ and the like groups; and alkyl substituted with a non-interfering substituent, such as hydroxy, halogen, alkoxy or nitro; corresponding alkoxy groups.

When R8 is aryl or the corresponding aryloxy group, it typically contains from about 5 to about 14 carbon atoms, or about 5 to about 12 carbon atoms, and optionally, may contain one or more rings that are fused to each other. Examples of particularly suitable aromatic groups include phenyl, phenoxy, naphthyl, anthracenyl, phenanthrenyl and the like.

When R8 is heterocyclic or heterocycloxy, it typically contains from about 5 to about 14 ring atoms, alternatively from about 5 to about 12 ring atoms, and one or more heteroatoms. Examples of suitable heterocyclic groups include furan, thiophene, pyrrole, isopyrrole, 3-isopyrrole, pyrazole, 2-isoimidazole, 1,2,3-triazole, 1,2,4-triazole, oxazole, thiazole, isothiazole, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-dioxazole, 1,2,4-dioxazole, 1,3,2-dioxazole, 1,3,4-dioxazole, 1,2,5-oxatriazole, 1,2-pyran, 1,4-pyran, 1,2-pyrone, 1,4-pyrone, 1,2-dioxin, 1,3-dioxin, pyridine, N-alkyl pyridinium, pyridazine, pyrimidine, pyrazine, 1,3,5-triazine, 1,2,4-triazine, 1,2,3-triazine, 1,2-oxazine, 1,3-oxazine, 1,4-oxazine, o-isoxazine, p-isoxazine, 1,2,5-oxathiazine, 1,2,6-oxathiazine, 1,4,2-oxadiazine, 1,3,5-oxadiazine, azepine, oxepin, thiepin, indene, isoindene, benzofuran, isobenzofuran, thionaphthene, isothionaphthene, indole, indolenine, 2-isobenzazole, isoindazole, indoxazine, benzoxazole, anthranil, 1,2-benzopyran, 1,2-benzopyrone, 1,4-benzopyrone, 2,1 -benzopyrone, 2,3 -benzopyrone, quinoline, isoquinoline, 12, -benzodiazine, 1,3 -benzodiazine, naphthyridine, pyrido-[3,4-b]-pyridine, pyrido-[3,2-b]-pyridine, pyrido-[4,3-b]-pyridine, 1,3,2-benzoxazine, 1,4,2-benzoxazine, 2,3,1-benzoxazine, 3,1,4-benzoxazine, 1,2-benzisoxazine, 1,4-benzisoxazine, carbazole, xanthrene, acridine, purine, and the like. In certain embodiments, when R8 is heterocyclic or heterocycloxy, it is selected from the group consisting of furan, pyridine, N-alkylpyridine, 1,2,3- and 1,2,4-triazoles, indene, anthracene and purine rings.

In certain embodiments, R8 is an alkyl group, an alkoxy group, a phenyl group, a phenoxy group, a heterocycloxy group, or an ethoxy group.

In still other embodiments, R8, such as an alkyl, may be conjugated to a bioactive substance to form a pendant drug delivery system.

In certain embodiments, the number of monomeric units in Formula V and other subject formulas that make up the subject polymers ranges over a wide range, e.g., from about 5 to 25,000 or more, but generally from about 100 to 5000, or 10,000. Alternatively, in other embodiments, the number of monomeric units may be about 10, 25, 50, 75, 100, 150, 200, 300 or 5400.

In Formula V and other formulas herein, “*” represents other monomeric units of the subject polymer, which may be the same or different from the unit depicted in the formula in question, or a chain terminating group, by which the polymer terminates. Examples of such chain terminating groups include monoflnctional alcohols and amines.

In another aspect, the polymeric compositions of the present invention include one or more recurring monomeric units represented in general Formula VI:

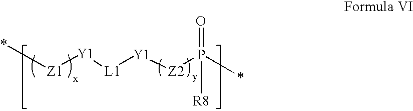

wherein Z1 and Z2, respectively, for each independent occurrence is:

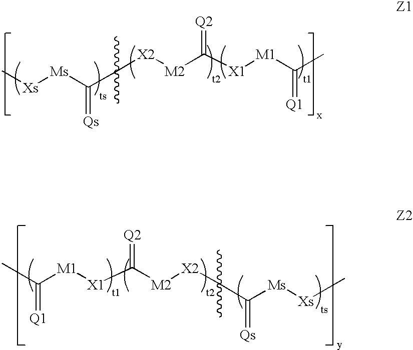

wherein, independently for each occurrence set forth above:

Q1, Q2 . . . Qs, each independently, represent O or N(R1);

X1, X2 . . . Xs, each independently, represent —O— or —N(R1);

the sum of t1, t2 . . . ts is an integer and at least one or more;

Y1 represents —O—, —S— or —N(R7)-;

x and y are each independently integers from 1 to about 1000 or more;

L1 and M1, M2 . . . Ms each independently, represent the moieties discussed below; and

the other moieties are as defined above.

M1, M2 . . . Ms (collectively, M) in Formula VI are each independently any chemical moiety that does not materially interfere with the polymerization, biocompatibility or biodegradation (or any combination of those three properties) of the subject polymer. For certain embodiments, M in the formula are each independently: (i) a branched or straight chain aliphatic or aryl group having from 1 to about 50 carbon atoms, or (ii) a branched or straight chain, oxa-, thia-, or aza-aliphatic group having from 1 to about 50 carbon atoms, both optionally substituted. In certain embodiments, the number of such carbon atoms does not exceed 20. In other embodiments, M may be any divalent aliphatic moiety having from 1 to about 20 carbon atoms, including therein from 1 to about 7 carbon atoms.

M may include an aromatic or heteroaromatic moiety, optionally with non-interfering substituents. In certain embodiments, none of the atoms (usually but not always C) that form the cyclic ring that gives rise to the aromatic moiety are part of the polymer backbone chain.

Specifically, when M is a branched or straight chain aliphatic group having from 1 to about 20 carbon atoms, it may be, for example, an alkylene group such as methylene, ethylene, 1-methylethylene, 1,2-dimethylethylene, n-propylene, trimethylene, isopropylene, 2,2-dimethylpropylene, n-pentylene, n-hexylene, n-heptylene, n-octylene, n-nonylene, n-decylene, n-undecylene, n-dodecylene, and the like; an alkenylene group such as n-propenylene, 2-vinylpropylene, n-butenylene, 3-thexylbutylene, n-pentenylene, 4-(3-propenyl)hexylene, n-octenylene, 1-(4-butenyl)-3-methyldecylene, 2-(3-propenyl)dodecylene, hexadecenylene and the like; an alkynylene group, such as ethynylene, propynylene, 3-(2-ethynyl)pentylene, n-hexynylene, 2-(2-propynyl)decylene, and the like; or any alkylene, alkenylene or alkynylene group, including those listed above, substituted with a materially non-interfering substituent, for example, a hydroxy, halogen or nitrogen group, such as 2-chloro-n-decylene, 1-hydroxy-3-ethenylbutylene, 2-propyl-6-nitro-10-dodecynylene, and the like. Other M of the present invention include —(CH₂)₃—, —(CH₂)₅— and (CH₂)₂OCH₂—.

When M is a branched or straight chain oxaaliphatic group having from 1 to about 20 carbon atoms, it may be, for example, a divalent alkoxylene group, such as ethoxylene, 2-methylethoxylene, propoxylene, butoxylene, pentoxylene, dodecyloxylene, hexadecyloxylene, and the like. When M is a branched or straight chain oxaaliphatic group, it may have the formula —(CH₂)ₐ—O—(CH₂)_{b}— wherein each of a and b, independently, is about 1 to about 7.

When M is a branched or straight chain oxaaliphatic group having from 1 to about 20 carbon atoms, it may also be, for example, a dioxaalkylene group such as dioxymethylene, dioxyethylene, 1,3-dioxypropylene, 2-methoxy-1,3-dioxypropylene, 1,3-dioxy-2-methylpropylene, dioxy-n-pentylene, dioxy-n-octadecylene, methoxylene-methoxylene, ethoxylene-methoxylene, ethoxylene-ethoxylene, ethoxylene-l-propoxylene, butoxylene-n-propoxylene, pentadecyloxylene-methoxylene, and the like. When M is a branched or straight chain, dioxyaliphatic group, it may have the formula —(CH₂)ₐ—O—(CH₂)_{b}—O—(CH₂)_{c}—, wherein each of a, b, and c is independently from 1 to about 7.

When M is a branched or straight chain thiaaliphatic group, the group may be any of the preceding oxaaliphatic groups wherein the oxygen atoms are replaced by sulfur atoms.

When M is a branched or straight chain, aza-aliphatic group having from 1 to about 20 carbon atoms, it may be a divalent group such as —CH₂NH—, —(CH₂)₂N—, —CH₂(C₂H₅)N—, -n-C₄H₉NH—, -t-C₄H₉NH—, —CH₂(C₃H₇)N—, —C₂H₅(C₂H₅)N—, —CH₂(C₈H₁₇)N—, —(CH₂)₂NHCH₂—, —(CH₂)₂NCH₂—, —CH(C₂H₅)NCH₂CH₂—, —n—C₄H₉NHCH₂—, —t—C₄H₉NHCH₂CH₂—, —CH₂(C₃H₇)N(CH₂)₄—, —C₂H₅(C₂H₅)NCH₂—, —CH₂(C₈H₁₇)NCH₂CH₂—, and the like. When M is a branched or straight chain, amino-aliphatic group, it may have the formula —(CH₂)ₐNR1- or —(CH₂)ₐN(R1)(CH₂)_{b}- where R1 is —H, aryl, alkenyl or alkyl and each of a and b is independently from about 1 to about 7.

x and y of Formula VI each independently represent integers in the range of about 1 to about 1000, e.g., about 1, about 10, about 20, about 50, about 100, about 250, about 500, about 750, about 1000, etc.

For Formula VI, the average molar ratio of (x or y):L1, assuming ts is equal to one, may vary greatly, typically between about 75:1 and about 2:1. In certain embodiments, the average molar ratio of (x or y):L1, when ts is equal to one, is about 10:1 to about 4:1, and preferably about 5:1. The molar ratio of x:y may also vary; typically, such ratio is about 1. Other possible embodiments may have ratios of 0.1, 0.25, 0.5, 0.75, 1.5, 2, 3, 4, 10 and the like.

A number of different polymer structures are contemplated by Formula VI. For example, in certain polymers exemplified by Formula VI, when the sum of t1, t2 . . . ts equals one for each of Z1 and Z2 and Q, M and X for each subunit ts are the same, then Formula VI becomes the following Formula VIa:

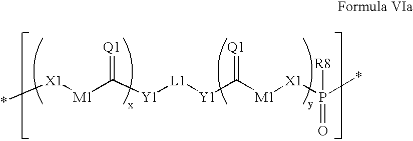

In certain embodiments of Formula VIa (and other subject formulas), x and y may be even integers.

The above Formula VI (and all of the subject formulae and polymers) encompass a variety of different polymer structures, including block copolymers, random copolymers, random terpolymers and segmented block copolymers and terpolymers. Additional structures for Z of subject monomeric units are set forth below, which exemplify in part the variety of structures contemplated by the present invention:

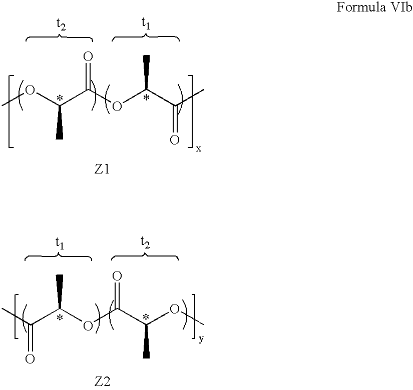

In Formula VIb (and other formulas described below), there may be more ts subunits depicted of the same molecular identity of those depicted in the formulas. For example, in Formula VIb, subunits t₁, and t₂ may be repeated in a sequence, e.g., alternating, in blocks (which may themselves repeat), or in any other pattern or random arrangement. Each subunit may repeat any number of times, and one subunit (e.g., t₁) may occur with substantially the same frequency, more often, or less often than another subunit (e.g., t₂), such that both subunits may be present in approximately the same amount, or in differing amounts, which may differ slightly or be highly disparate, e.g., one subunit is present nearly to the exclusion of the other. In certain embodiments, the chiral centers of each subunit may be the same or different and may be arranged in an orderly fashion or in a random sequence in each of Z1 and Z2.

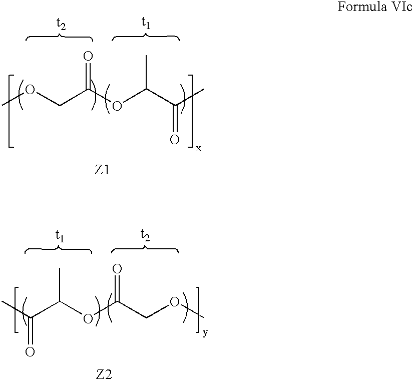

In certain embodiments of Formula VIc, the sum of the number of ts subunits in each of Z1 and Z2 is an even integer. As in other examples of Z1 and Z2, such as described above for Formula VIb, the ts subunits may be distributed randomly or in an ordered arrangement in each of Z1 or Z2.

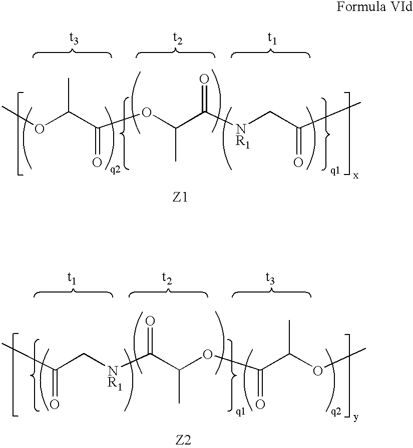

In Formula VId, the subunit q1 is comprised of two ts subunits, which may be repeated and arranged as described above for Formula VIb. In certain embodiments, q2 is an even integer, and in other embodiments, the subunits q1 and q2 may be distributed randomly or in an ordered pattern in each of Z1 and Z2. For example, subunits q1 and q2 may be repeated in a sequence, e.g., alternating, in blocks (which may themselves repeat), or in any other pattern or random arrangement. Each subunit may repeat any number of times, and one subunit (e.g., q₁) may occur with substantially the same frequency, more often, or less often than another subunit (e.g., q₂), such that both subunits may be present in approximately the same amount, or in differing amounts, which may differ slightly or be highly disparate, e.g., one subunit is present nearly to the exclusion of the other.

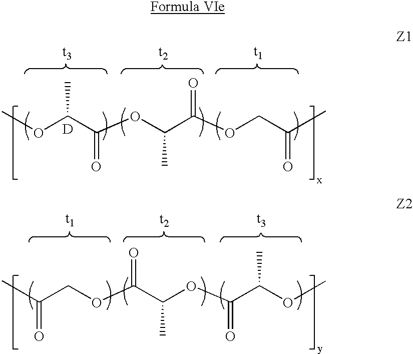

In certain embodiments of Formula VIe, the sum of the ts subunits for each of Z1 and Z2 is an even integer. In other embodiments, the each of the subunits t1, t₂, and t₃ may be distributed randomly or in an ordered arrangement in each of Z1 and Z2. For example, in Formula VIe, subunits t₁, t₂, and t₃ may be repeated in a sequence, e.g., alternating, in blocks (which may themselves repeat), or in any other pattern or random arrangement. Each subunit may repeat any number of times, and one subunit (e.g., t₁) may occur with substantially the same frequency, more often, or less often than another subunit (e.g., t₃), such that the three subunits may be present in approximately the same amount, or in differing amounts, which may differ slightly or be highly disparate, e.g., two subunits are present nearly to the exclusion of the third.

In certain embodiments of Formula VI, in which Q, M and X for each subunit are the same, Q1 represents O, M represents a lower alkylene group, and X1 represents O or S, preferably O. For example, M may represent —CH(CH₃)— to result in a polymer of Formula VI having a structure represented in Formula VIf:

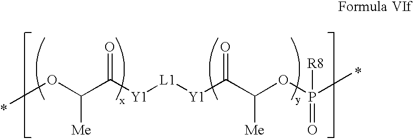

In certain embodiments of Formula VIf, as further described in the Exemplification below, L1 represents a lower alkylene chain, such as ethylene, propylene, etc. In certain embodiments, all Y1's represent O. In certain embodiments, R8 represents —O-lower alkyl, such as —OEt.

In certain embodiments of polymers depicted by Formula VI, the chirality of each subunit is identical, whereas in other embodiments, the chirality is different. By way of example but not limitation, in Formula VIb above, if the chiral centers of all of the subunits are D-enantiomers or L-enantiomers, then the monomeric unit is effectively equivalent to D-lactic acid or L-lactic acid, respectively, thereby giving rise to a region similar to poly(D-lactic acid) or poly-(L-lactic acid), respectively. Conversely, if the two subunits in Formula VIb are comprised of alternating D- and L-enantiomers (e.g., one unit of D-enantiomer, one unit of L-enantiomer, etc.), then the resulting polymeric region is analogous to poly(meso-lactic acid) (i.e., a polymer formed by polymerization of meso-lactide).

Finally, in certain embodiments of the monomeric units set forth in Formula VI, in which the entire polymer may or may not be composed of such units, the following moieties for Y1, L1, R8 Qs, Xs and Ms may be used (with a variety of different x and y being possible):

| Abbreviation | All Y1's | L1 | R8 |
|---|---|---|---|
| L-PL(EG)EOP | O | —CH₂CH₂— | —OCH₂CH₃ |
| L-PL(EG)HOP | O | —CH₂CH₂— | —O(CH₂)₅CH₃ |
| D,L-PL(EG)EOP* | O | —CH₂CH₂— | —OCH₂CH₃ |
| D,L-PL(PG)EOP* | O | —CH₂(CH₃)CH₂— | —OCH₂CH₃ |
| D-PL(PG)EOP | O | —CH₂(CH₃)CH₂— | —OCH₂CH₃ |
| L-PL(PG)EOP | O | —CH₂(CH₃)CH₂— | —OCH₂CH₃ |
| D,L-PL(HD)EOP* | O | 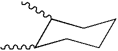 | —OCH₂CH₃ |
| D,L-PL(PG)HOP* | O | —CH₂(CH₃)CH₂— | —O(CH₂)₅CH₃ |
| D,L-PL(PG)EP* | O | —CH₂(CH₃)CH₂— | —CH₂CH₃ |

| | All | All | | |
|---|---|---|---|---|
| Abbreviation | Qs | Xs | M1 | M2 |
| L-PL(EG)EOP | O | O | —CH(CH₃)-(L) | N/A |
| L-PL(EG)HOP | O | O | —CH(CH₃)-(L) | N/A |
| D,L-PL(EG)EOP* | O | O | —CH(CH₃)-(L or D) | —CH(CH₃)-(D |
| | | | | or L) |
| D,L-PL(PG)EOP* | O | O | —CH(CH₃)-(L or D) | —CH(CH₃)-(D |
| | | | | or L) |
| D-PL(PG)EOP | O | O | —CH(CH₃)-(D) | N/A |
| L-PL(PG)EOP | O | O | —CH(CH₃)-(L) | N/A |
| D,L-PL(HD)EOP* | O | O | —CH(CH₃)-(L or D) | —CH(CH₃)-(L |
| | | | | or D) |
| D,L-PL(PG)HOP* | O | O | —CH(CH₃)-(L or D) | —CH(CH₃)-(L |
| | | | | or D) |
| D,L-PL(PG)EP* | O | O | —CH(CH₃)-(L or D) | —CH(CH₃)-(L |
| | | | | or D) |

*For D,L-PL(EG)EOP, D,L-PL(PG)EOP, D,L-PL(HD)EOP, D,L-PL(PG)HOP, and D,L-PL(PG)EP, if the chiral carbon of M1 has configuration L, then M2 will have configuration D, and vice-versa. The order of the chiral centers in each subunit M1 and M2 for each Z1 and Z2 will be in random order.

In addition to the particular chiral version of the subject polymers described in the above table, polymers in which the chirality of Ms varies in each subunit M in the subject polymers are also possible. For instance, referring to D,L-PL(EG)EOP by example, a random order of D and L, in varying amounts, are possible for this polymer. In contrast, the table sets forth one such example in which a D and L chiral M are always adjacent, in equal amounts, but that need not always be the case.

In another embodiment of the present invention, the polymeric compositions of the present invention include one or more recurring monomeric units represented in general Formula VII:

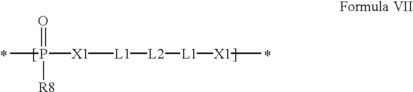

wherein, independently for each occurrence:

L2 is a divalent organic group as described in greater detail below; and

the other moieties are as defined as above.

In Formula VII, L2 may be a divalent, branched or straight chain aliphatic group, a cycloaliphatic group, or a group of the formula:

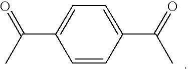

Specific examples of particular divalent, branched or straight chain aliphatic groups include an alkylene group with 1 to 7 carbon atoms, such as 2-methylpropylene or ethylene. Specific examples of cycloaliphatic groups include cycloalkylene groups, such as cyclopentylene, 2-methylcyclopentylene, cyclohexylene and 2-chloro-cyclohexylene; cycloalkenylene groups, such as cyclohexenylene; and cycloalkylene groups having fused or bridged additional ring structures, such as tetralinylene, decalinylene and norpinanylene; or the like.

In certain embodiments of the monomeric units set forth in Formula VII, in which the entire polymer may or may not be composed of such units, the following moieties for X1, L1 and R8 may be used:

| Abbreviation | All X1 | All L1 | L2 | R8 |
|---|---|---|---|---|
| P(trans-CHDM/HOP) | O | —CH₂— | 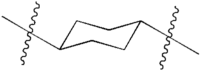 | —O(CH₂)₅CH₃ |
| | | | trans-1,4-cyclohexyl | |
| P(cis- and trans-CHDM/HOP) | O | —CH₂— | mixture of trans-1,4-cyclohexyl and | —O(CH₂)₅CH₃ |
| | | | 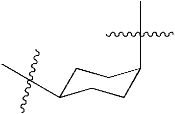 | |
| | | | cis-1,4-cyclohexyl | |
| P(trans-CHDM/BOP) | O | —CH₂— | trans-1,4-cyclohexyl | —O(CH₂)₃CH₃ |
| P(trans-CHDM/EOP) | O | —CH₂— | trans-1,4-cyclohexyl | —OCH₂CH₃ |

In another embodiment of the present invention, the polymeric compositions of the present invention include one or more recurring monomeric units represented in general Formula VIII:

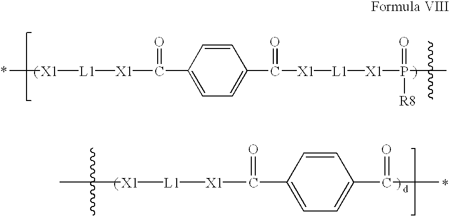

wherein, independently for each occurrence, d is equal to one or more, and optionally two, x is equal to or greater than one, and all of the other moieties are as defined above. In certain embodiments of Formula VIII, each of L1 independently may be an alkylene group, a cycloaliphatic group, a phenylene group or a divalent group of the formula:

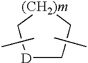

wherein D is O, N or S and m is 0 to 3. Alternatively, L1 is a branched or straight chain alkylyene group having from 1 to 7 carbon atoms, such as a methylene, ethylene, n-propylene, 2-methylpropylene, 2,2′-dimethylpropylene group and the like.

In certain embodiments of the monomeric units set forth in Formula VIII, in which the entire polymer may or may not be composed of such units, the following moieties for X1, L1 and R8 may be used (with a variety of different x possible for each example and with d preferably equal to two):

| Abbreviation | All X1 | All L1 | R8 |
|---|---|---|---|
| P(BHET-EOP/TC) | O | -CH₂CH₂- | -OCH₂CH₃ |
| P(BHDPT-EOP/TC) | O | -CH₂CH(CH₃)₂CH₂- | -OCH₂CH₃ |
| P(BHDPT-HOP/TC) | O | -CH₂CH(CH₃)₂CH₂- | -OC₆H₁₃ |
| P(BHPT-EOP/TC) | O | -CH₂CH₂CH₂- | -OCH₂CH₃ |
| P(BNMPT-EOP/TC) | O | CH₂CH₂(CH₃)CH₂- | -OCH₂CH₃ |

In Formula VIII, the aryl groups represented therein may be substituted with a non-interfering substituent, for example, a hydroxy-, halogen-, or nitrogen-substituted moiety.

Other phosphorus containing polymers which may be adapted for use in the subject invention, and methods of making the same, are described in the art, including those described in U.S. Pat. Nos. 5,256,765 and 5,194,581; PCT publications WO 98/44020, WO 98/44021, and WO 98/48859; and U.S. applications Ser. Nos. 09/053,649, 09/053,648 and 09/070,204. For all of the above-identified groups, non-interfering substituents may also be present.

In certain embodiments, the polymers are comprised almost entirely, if not entirely, of the same subunit. Alternatively, in other embodiments, the polymers may be copolymers, in which different subunits and/or other monomeric units are incorporated into the polymer. In certain instances, the polymers are random copolymers, in which the different subunits and/or other monomeric units are distributed randomly throughout the polymer chain. For example, the polymer having units of Formula V may consist of effectively only one type of such subunit, or alternatively two or more types of such subunits. In addition, the polymer may contain monomeric units other than those subunits represented by Formula V.

In other embodiments, the different types of monomeric units, be they one or more subunits depicted by the subject formulas or other monomeric units, are distributed randomly throughout the chain. In part, the term “random” is intended to refer to the situation in which the particular distribution or incorporation of monomeric units in a polymer that has more than one type of monomeric units is not directed or controlled directly by the synthetic protocol, but instead results from features inherent to the polymer system, such as the reactivity, amounts of subunits and other characteristics of the synthetic reaction or other methods of manufacture, processing or treatment.

In certain embodiments, the subject polymers may be cross-linked. For example, substituents of the polymeric chain, may be selected to permit additional inter-chain cross-linking by covalent or electrostatic (including hydrogen-binding or the formation of salt bridges), e.g., by the use of a organic residue appropriately substituted.

The ratio of different subunits in any polymer as described above may vary. For example, in certain embodiments, polymers may be composed almost entirely, if not entirely, of a single monomeric element, such as a subunit depicted in Formula V. Alternatively, in other instances, the polymers are effectively composed of two different subunits, in which the percentage of each subunit may vary from less than 1:99 to more than 99:1, or alternatively 10:90, 15:85, 25:75, 40:60, 50:50, 60:40, 75:25, 85:15, 90:10 or the like. For example, in some instances, a polymer may be composed of two different subunits that may be both represented by the generic Formula V, but which differ in their chemical identity. In certain embodiments, the polymers may have just a few percent, or even less (for example, about 5, 2.5, 1, 0.5, 0.1%) of the subunits having phosphorous-based linkages. In other embodiments, in which three or more different monomeric units are present, the present invention contemplates a range of mixtures like those taught for the two-component systems.

In certain embodiments, the polymeric chains of the subject compositions, e.g., which include repetitive elements shown in any of the subject formulas, have molecular weights ranging from about 2000 or less to about 1,000,000 or more daltons, or alternatively about 10,000, 20,000, 30,000, 40,000, or 50,000 daltons, more particularly at least about 100,000 daltons, and even more specifically at least about 250,000 daltons or even at least 500,000 daltons. Number-average molecular weight (Mn) may also vary widely, but generally fall in the range of about 1,000 to about 200,000 daltons, preferably from about 1,000 to about 100,000 daltons and, even more preferably, from about 1,000 to about 50,000 daltons. Most preferably, Mn varies between about 8,000 and 45,000 daltons. Within a given sample of a subject polymer, a wide range of molecular weights may be present. For example, molecules within the sample may have molecular weights which differ by a factor of 2, 5, 10, 20, 50, 100, or more, or which differ from the average molecular weight by a factor of 2, 5, 10, 20, 50, 100, or more.

One method to determine molecular weight is by gel permeation chromatography (“GPC”), e.g., mixed bed columns, CH₂Cl₂ solvent, light scattering detector, and off-line dn/dc. Other methods are known in the art.

In certain embodiments, the intrinsic viscosities of the polymers generally vary from about 0.01 to about 2.0 dL/g in chloroform at 40° C., alternatively from about 0.01 to about 1.0 dL/g and, occasionally, from about 0.01 to about 0.5 dL/g.

The glass transition temperature (Tg) of the subject polymers may vary widely, and depend on a variety of factors, such as the degree of branching in the polymer components, the relative proportion of phosphorous-containing monomer used to make the polymer, and the like. When the article of the invention is a rigid solid, the Tg is often within the range of from about −10° C. to about 80° C., particularly between about 0 and 50° C. and, even more particularly between about 25° C. to about 35° C. In other embodiments, the Tg is preferably low enough to keep the composition of the invention flowable at body temperature. Then, the glass transition temperature of the polymer used in the invention is usually about 0 to about 37° C., or alternatively from about 0 to about 25° C.

In certain embodiments, substituents of the phosphorus atom, such as R8 in the above formulas, and other components of the subject polymers may permit additional inter-chain cross-linking by covalent or electrostatic interactions (including, for example, hydrogen-binding or the formation of salt bridges) by having a side chain of either of them appropriately substituted as discussed in greater detail below.

In other embodiments, the polymer composition of the invention may be a flexible or flowable material. When the polymer used is itself flowable, the polymer composition of the invention, even when viscous, need not include a biocompatible solvent to be flowable, although trace or residual amounts of biocompatible solvents may still be present.

A flowable polymer composition may be especially suitable for instillation within an irregular body cavity or space, such as that resulting from removal of a neoplastic growth. A flowable material is often capable of assuming the shape of the contours of such space so that it can be applied in certain regions initially and flow therefrom to coat the tissue. A flowable polymer may be particularly adapted for instillation through a needle, catheter or other delivery device such as a laparascope, since its flowable characteristics allow it to reach surfaces that extend beyond the immediate reach of the delivery device. Physical properties of polymers may be adjusted to achieve a desirable state of fluidity or flowability by modification of their chemical components and crosslinking, using methods familiar to practitioners of ordinary skill in the art.

A flexible polymer may be used in the fabrication of a solid article. Flexibility involves having the capacity to be repeatedly bent and restored to its original shape. Solid articles made from flexible polymers are adapted for placement in body cavities where they will encounter the motion of adjacent organs or body walls. A flexible solid article can thus be sufficiently deformed by a motile organ structure that it does not cause tissue damage. Flexibility is particularly advantageous where a solid article might be dislodged from its original position and thereby encounter an unanticipated moving structure; flexibility may allow the solid article to bend out of the way of the moving structure instead of injuring it. Physical properties of polymers may be adjusted to attain a desirable degree of flexibility by modification of the chemical components and crosslinking thereof, using methods familiar to practitioners of ordinary skill in the art.

While it is possible that the subject polymer or the biologically active agent may be dissolved in a small quantity of a solvent that is non-toxic to more efficiently produce an amorphous, monolithic distribution or a fine dispersion of the biologically active agent in the flexible or flowable composition, it is an advantage of the invention that, in a preferred embodiment, no solvent is needed to form a flowable composition. Moreover, the use of solvents is preferably avoided because, once a polymer composition containing solvent is placed totally or partially within the body, the solvent dissipates or diffuses away from the polymer and must be processed and eliminated by the body, placing an extra burden on the body's clearance ability at a time when the illness (and/or other treatments for the illness) may have already deleteriously affected it.

However, when a solvent is used to facilitate mixing or to maintain the flowability of the polymer composition of the invention, it should be non-toxic, otherwise biocompatible, and should be used in relatively small amounts. Solvents that are toxic clearly should not be used in any material to be placed even partially within a living body. Such a solvent also must not cause substantial tissue irritation or necrosis at the site of administration.

Examples of suitable biocompatible solvents, when used, include N-methyl-2-pyrrolidone, 2-pyrrolidone, ethanol, propylene glycol, acetone, methyl acetate, ethyl acetate, methyl ethyl ketone, dimethylformamide, dimethyl sulfoxide, tetrahydrofuran, caprolactam, dimethyl-sulfoxide, oleic acid, or 1-dodecylazacycloheptan-2-one. Preferred solvents include N-methyl-2-pyrrolidone, 2-pyrrolidone, dimethyl sulfoxide, and acetone because of their solvating ability and their biocompatibility.

Microspheres of the subjetc compositions may be manufactured by incorporating the drug into the polymer matrix by either dissolving or suspending the drug into polymer solution and the mixture will be subsequently dried by techniques familiar to those skill in the arts to form microspheres. These techniques include but not limited to spray drying, coating, various emulsion methods and supercritical fluid processing. The microspheres may be mixed with a pharmaceutically acceptable diluent prior to the administration for injection. They may also be directly applied to the desired site, such as a surgical wound or cavity, by various delivery systems including pouring and spraying. The microspheres may also be mixed with pharmaceutically acceptable ingredients to create ointment or cream for topical applications.

C. Therapeutic Compositions

The radiosensitizers of the present invention are used in amounts that are therapeutically effective, which varies widely depending largely on the particular radiosensitizer being used. The amount of radiosensitizer incorporated into the composition also depends upon the desired release profile, the concentration of the agent required for a biological effect, and the length of time that the agent should be released for treatment. In certain embodiments, the agent may be blended with the polymer matrix of the invention at different loading levels, preferably at room temperature and without the need for an organic solvent. In other embodiments, the compositions of the present invention may be formulated as microspheres or pressed as rods.

There is no critical upper limit on the amount of radiosensitizer incorporated except for that of an acceptable solution or dispersion viscosity to maintain the physical characteristics desired for the composition. The lower limit of the radiosensitizer incorporated into the subject polymer is dependent upon the activity of the agent and the length of time needed for treatment. Thus, the amount of the radiosensitizer should not be so small that it fails to produce the desired physiological effect, nor so large that the radiosensitizer is released in an uncontrollable manner. Typically, within these limits, amounts of the radiosensitizer from about 5% up to about 60% may be incorporated into the subject compositions. However, lesser amounts may be used to achieve efficacious levels of treatment for radiosensitizer that are particularly potent.

In addition, the polymer composition of the invention may comprise blends of the polymer of the invention with other biocompatible polymers or copolymers, so long as the additional polymers or copolymers do not interfere undesirably with the biodegradable or mechanical characteristics of the composition. Blends of the polymer of the invention with such other polymers may offer even greater flexibility in designing the precise release profile desired for targeted drug delivery or the precise rate of biodegradability desired. Examples of such additional biocompatible polymers include other poly(phosphoesters), poly(carbonates), poly(esters), poly(orthoesters), poly(amides), poly(urethanes), poly(imino-carbonates), and poly(anhydrides).

For delivery of an radiosensitizer or some other biologically active substance, the agent or substance is added to the polymer composition. A variety of methods are known in the art for encapsulating a biologically active substance in a polymer. For example, the agent or substance may be dissolved to form a homogeneous solution of reasonably constant concentration in the polymer composition, or it may be dispersed to form a suspension or dispersion within the polymer composition at a desired level of “loading” (grams of biologically active substance per grams of total composition including the biologically active substance, usually expressed as a percentage).

In part, a subject polymer composition of the present invention includes both: (a) a radiosensitizer, and (b) a biocompatible polymer, optionally biodegradable, such as one having the recurring monomeric units shown in one of the foregoing formulas, or any other biocompatible polymer mentioned above or known in the art.

In addition to radiosensitizer, the subject compositions may contain a “drug”, “therapeutic agent”, “medicament” or “bioactive substance”, which are biologically, physiologically, or pharmacologically active substances that act locally or systemically in the human or animal body. For example, a subject composition may include an augmenting agent, as discussed above. Various forms of the medicaments or biologically active materials may be used which are capable of being released from the polymer matrix into adjacent tissues or fluids. They may be acidic, basic, or salts. They may be neutral molecules, polar molecules, or molecular complexes capable of hydrogen bonding. They may be in the form of ethers, esters, amides and the like, which are biologically activated when injected into the human or animal body. An radiosensitizer is also an example of a “bioactive substance.”

Any additional bioactive substance in a subject composition may vary widely with the purpose for the composition. The term bioactive agent includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

Plasticizers and stabilizing agents known in the art may be incorporated in polymers of the present invention. In certain embodiments, additives such as plasticizers and stabilizing agents are selected for their biocompatibility.

A composition of this invention may further contain one or more adjuvant substances, such as fillers, thickening agents or the like. In other embodiments, materials that serve as adjuvants may be associated with the polymer matrix. Such additional materials may affect the characteristics of the polymer matrix that results. For example, fillers, such as bovine serum albumin (BSA) or mouse serum albumin (MSA), may be associated with the polymer matrix. In certain embodiments, the amount of filler may range from about 0.1 to about 50% or more by weight of the polymer matrix, or about 2.5, 5, 10, 25, 40 percent. Incorporation of such fillers may affect the biodegradation of the polymeric material and/or the sustained release rate of any encapsulated substance. Other fillers known to those of skill in the art, such as carbohydrates, sugars, starches, saccharides, celluoses and polysaccharides, including mannitose and sucrose, may be used in certain embodiments in the present invention.

In other embodiments, spheronization enhancers facilitate the production of subject polymeric matrices that are generally spherical in shape. Substances such as zein, microcrystalline cellulose or microcrystalline cellulose co-processed with sodium carboxymethyl cellulose may confer plasticity to the subject compositions as well as implant strength and integrity. In particular embodiments, during spheronization, extrudates that are rigid, but not plastic, result in the formation of dumbbell shaped implants and/or a high proportion of fines, and extrudates that are plastic, but not rigid, tend to agglomerate and form excessively large implants. In such embodiments, a balance between rigidity and plasticity is desirable. The percent of spheronization enhancer in a formulation depends on the other excipient characteristics and is typically in the range of 10-90% (w/w).

In certain embodiments, a subject composition includes an excipient. A particular excipient may be selected based on its melting point, solubility in a selected solvent (e.g., a solvent which dissolves the polymer and/or the radiosensitizer), and the resulting characteristics of the microparticles. A list of exemplary excipients include ethyl cellulose, cholesterol, potassium stearate, docusate, mannitol, NaCl, benzoic acid, tartaric acid, sorbic acid, PEG 20,000 (and other forms of PEG), zinc stearate and magnesium stearate.

Buffers, acids and bases may be incorporated in the subject compositions to adjust their pH. Agents to increase the diffusion distance of agents released from the polymer matrix may also be included.

Disintegrants are substances which, in the presence of liquid, promote the disruption of the subject compositions. Disintegrants are most often used in implants, in which the function of the disintegrant is to counteract or neutralize the effect of any binding materials used in the subject formulation. In general, the mechanism of disintegration involves moisture absorption and swelling by an insoluble material. Examples of disintegrants include croscarmellose sodium and crospovidone which, in certain embodiments, may be incorporated into the polymeric matrices in the range of about 1-20% of total matrix weight. In other cases, soluble fillers such as sugars (mannitol and lactose) can also be added to facilitate disintegration of the subject composition upon use.

Other materials may be used to advantage to control the desired release rate of a therapeutic agent for a particular treatment protocol. For example, if the sustained release is too slow for a particular application, a pore-forming agent may be added to generate additional pores in the matrix. Any biocompatible water-soluble material may be used as the pore-forming agent. They may be capable of dissolving, diffusing or dispersing out of the formed polymer system whereupon pores and microporous channels are generated in the system. The amount of pore-forming agent (and size of dispersed particles of such pore-forming agent, if appropriate) within the composition should affect the size and number of the pores in the polymer system.

Pore-forming agents include any pharmaceutically acceptable organic or inorganic substance that is substantially miscible in water and body fluids and will dissipate from the forming and formed matrix into aqueous medium or body fluids or water-immiscible substances that rapidly degrade to water-soluble substances. Suitable pore-forming agents include, for example, sugars such as sucrose and dextrose, salts such as sodium chloride and sodium carbonate, and polymers such as hydroxylpropylcellulose, carboxymethylcellulose, polyethylene glycol, and polyvinylpyrrolidone. The size and extent of the pores may be varied over a wide range by changing the molecular weight and percentage of pore-forming agent incorporated into the polymer system.

The charge, lipophilicity or hydrophilicity of any subject polymeric matrix may be modified by attaching in some fashion an appropriate compound to the surface of the matrix. For example, surfactants may be used to enhance wettability of poorly soluble or hydrophobic compositions. Examples of suitable surfactants include dextran, polysorbates and sodium lauryl sulfate. In general, surfactants are used in low concentrations, generally less than about 5%.

Binders are adhesive materials that may be incorporated in polymeric formulations to bind and maintain matrix integrity. Binders may be added as dry powder or as solution. Sugars and natural and synthetic polymers may act as binders. Materials added specifically as binders are generally included in the range of about 0.5%-15% w/w of the matrix formulation. Certain materials, such as microcrystalline cellulose, also used as a spheronization enhancer, also have additional binding properties.

Various coatings may be applied to modify the properties of the matrices. Three exemplary types of coatings are seal, gloss and enteric coatings. Other types of coatings having various dissolution or erosion properties may be used to further modify subject matrices behavior, and such coatings are readily known to one of ordinary skill in the art.

The present compositions may additionally contain one or more optional additives such as fibrous reinforcement, colorants, perfumes, rubber modifiers, modifying agents, etc. In practice, each of these optional additives should be compatible with the resulting polymer and its intended use. Examples of suitable fibrous reinforcement include PGA microfibrils, collagen microfibrils, cellulosic microfibrils, and olefinic microfibrils. The amount of each of these optional additives employed in the composition is an amount necessary to achieve the desired effect.

D. Physical Structures

The subject polymers may be formed in a variety of shapes. For example, in certain embodiments, subject polymer matrices may be presented in the form of microparticles or nanoparticles. Such particles may be prepared by a variety of methods known in the art, including for example, solvent evaporation, spray drying or double emulsion methods.

The shape of microparticles and nanoparticles may be determined by scanning electron microscopy. Spherically shaped nanoparticles are used in certain embodiments for circulation through the bloodstream. If desired, the particles may be fabricated using known techniques into other shapes that are more useful for a specific application.

In addition to intracellular delivery of a therapeutic agent, it also possible that particles of the subject compositions, such as microparticles or nanoparticles, may undergo endocytosis, thereby obtaining access to the cell. The frequency of such an endocytosis process will likely depend on the size of any particle.

In certain embodiments, solid articles useful in defining shape and providing rigidity and structural strength to the polymeric matrices may be used. For example, an polymer may be formed on a mesh or other weave for implantation.

The mechanical properties of the polymer may be important for the processability of making molded or pressed articles for implantation. For example, the glass transition temperature may vary widely but must be sufficiently lower than the temperature of decomposition to accommodate conventional fabrication techniques, such as compression molding, extrusion or injection molding.

E. Biodegradability and Release Characteristics

In certain embodiments, the polymers and blends of the present invention, upon contact with body fluids, undergo gradual degradation. The life of a biodegradable polymer in vivo depends, among other things, upon its molecular weight, crystallinity, biostability, and the degree of crosslinking. In general, the greater the molecular weight, the higher the degree of crystallinity, and the greater the biostability, the slower biodegradation will be.

If a subject polymer matrix is formulated with an radiosensitizer or other material, release of such an agent or other material for a sustained or extended period as compared to the release from an isotonic saline solution generally results. Such release profile may result in prolonged delivery (over, say 1 to about 2,000 hours, or alternatively about 2 to about 800 hours) of effective amounts (e.g., about 0.0001 mg/kg/hour to about 10 mg/kg/hour) of the radiosensitizer or any other material associated with the polymer.

A variety of factors may affect the desired rate of hydrolysis of polymers of the subject invention, the desired softness and flexibility of the resulting solid matrix, rate and extent of bioactive material release. Some of such factors include: the selection of the various substituent groups, such as the phosphate group making up the linkage in the polymer backbone (or analogs thereof), the enantiomeric or diastereomeric purity of the monomeric subunits, homogeneity of subunits found in the polymer, and the length of the polymer. For instance, the present invention contemplates heteropolymers with varying linkages, and/or the inclusion of other monomeric elements in the polymer, in order to control, for example, the rate of biodegradation of the matrix.

To illustrate further, a wide range of degradation rates may be obtained by adjusting the hydrophobicities of the backbones or side chains of the polymers while still maintaining sufficient biodegradability for the use intended for any such polymer. Such a result may be achieved by varying the various functional groups of the polymer. For example, the combination of a hydrophobic backbone and a hydrophilic linkage produces heterogeneous degradation because cleavage is encouraged whereas water penetration is resisted. In another example, it is expected that use of substituent on phosphate in the polymers of the present invention that is lipophilic, hydrophobic or bulky group would slow the rate of degradation. For example, it is expected that conversion of the phosphate side chain to a more lipophilic, more hydrophobic or more sterically bulky group would slow down the rate of biodegradation. Thus, release is usually faster from polymer compositions with a small aliphatic group side chain than with a bulky aromatic side chain.

One protocol generally accepted in the field that may be used to determine the release rate of any therapeutic agent or other material loaded in the polymer matrices of the present invention involves degradation of any such matrix in a 0.1 M PBS solution (pH 7.4) at 37° C., an assay known in the art. For purposes of the present invention, the term “PBS protocol” is used herein to refer to such protocol.

In certain instances, the release rates of different polymer systems of the present invention may be compared by subjecting them to such a protocol. In certain instances, it may be necessary to process polymeric systems in the same fashion to allow direct and relatively accurate comparisons of different systems to be made. For example, the present invention teaches several different means of formulating the polymeric matrices of the present invention. Such comparisons may indicate that any one polymeric system releases incorporated material at a rate from about 2 or less to about 1000 or more times faster than another polymeric system. Alternatively, a comparison may reveal a rate difference of about 3, 5, 7, 10, 25, 50, 100, 250, 500 or 750. Even higher rate differences are contemplated by the present invention and release rate protocols.

In certain embodiments, when formulated in a certain manner, the release rate for polymer systems of the present invention may present as mono- or bi-phasic. Release of any material incorporated into the polymer matrix, which is often provided as a microsphere, may be characterized in certain instances by an initial increased release rate, which may release from about 5 to about 50% or more of any incorporated material, or alternatively about 10, 15, 20, 25, 30 or 40%, followed by a release rate of lesser magnitude.

The release rate of any incorporated material may also be characterized by the amount of such material released per day per mg of polymer matrix. For example, in certain embodiments, the release rate may vary from about 1 ng or less of any incorporated material per day per mg of polymeric system to about 5000 or more ng/day.mg. Alternatively, the release rate may be about 10, 25, 50, 75, 100, 125, 150, 175, 200, 250, 300, 350, 400, 450, 500, 600, 700, 800 or 900 ng/day.mg. In still other embodiments, the release rate of any incorporated material may be 10,000 ng/day.mg or even higher. In certain instances, materials incorporated and characterized by such release rate protocols may include therapeutic agents, fillers, and other substances.

In another aspect, the rate of release of any material from any polymer matrix of the present invention may be presented as the half-life of such material in the such matrix.

In addition to the embodiment involving protocols for in vitro determination of release rates, in vivo protocols, whereby in certain instances release rates for polymeric systems may be determined in vivo, are also contemplated by the present invention. Other assays useful for determining the release of any material from the polymers of the present system are known in the art.

For certain of the subject compositions, the identity of the neoplasm or unwanted cell proliferation to be treated will affect the choice of composition to use, its loading level and release rate. For example, it is often desirable to tailor the rate of release to the rate of division of the tumor cells. For certain brain tumors, the rate of division of the proliferative cells is approximately every one to two weeks. Therefore, for the cells to be in the presence of the radiosensitizer for five cell cycles, the agent must be released over a 6 to 12 week time period. In contrast, the division time for small cell lung cancer is much less, and the desirable exposure period would therefore be correspondingly less.

F. Delivery Systems

In its simplest form, a biodegradable delivery system for an radiosensitizer consists of a dispersion of such a therapeutic agent in a polymer matrix. In other embodiments, an article is used for implantation, injection, or otherwise placed totally or partially within the body, the article comprising the subject compositions. It may be particularly particularly important that such an article result in minimal tissue irritation when applied to, implanted in or injected into vascularized tissue, hypovascularized post-operative tissue or tissue exposed to previous radiation. In certain embodiments, a solid, flowable or fluid article comprising the composition of the invention is inserted within an anatomic area by implantation, injection, endoscopy or otherwise being placed within an anatomic area of the subject being treated with a radiosensitizer.

As a structural medical device, the polymer compositions of the inventions provide a wide variety of physical forms having specific chemical, physical and mechanical properties suitable for insertion into an anatomic area.

The subject compositions may be delivered in a number of ways known to those of skill in the art that may increase their usefulness in different circumstances. For example, controlled release of radiosensitizers directly into the brain tumor area has the possible advantage of circumventing the blood-brain barrier. Likewise, intratumoral delivery of radiosensitizer and prolonged delivery of radiosensitizers in a depot form may also prove advantageous for treating different conditions or diseases, for reducing side effects, etc.

Drug delivery articles may be prepared in several ways. The polymer may be melt processed using conventional extrusion or injection molding techniques, or these products can be prepared by dissolving in an appropriate solvent, followed by formation of the device, and subsequent removal of the solvent by evaporation or extraction, e.g., by spray drying. By these methods, the polymers may be formed into articles of almost any size or shape desired, for example, implantable solid discs or wafers or injectable rods, microspheres, or other microparticles. Typical medical articles also include such as implants as laminates for degradable fabric or coatings to be placed on other implant devices.

In one embodiment, certain polymer compositions of the subject invention may be used to form a soft, drug-delivery “depot” that can be administered as a liquid, for example, by injection, but which remains sufficiently viscous to maintain the drug within the localized area around the injection site. By using a polymer composition in flowable form, even the need to make an incision can be eliminated. In any event, the flexible or flowable delivery “depot” will adjust to the shape of the space it occupies within the body with a minimum of trauma to surrounding tissues.

When the polymer composition of the invention is flexible or flowable, it may be placed anywhere within the body, including into a body cavity. It may be inserted into the body cavity through any of the access devices routinely used in the art to enter such cavities, for example, indwelling or acutely-inserted catheters, needles, chest tubes, peritoneal dialysis catheters and the like. A flowable or fluid polymer may be adapted for mixing with the exudate found within the body cavity with the diagnosis of cancer. A flowable or fluid polymer may be instilled in body cavities during surgery on organs therein to prevent subsequent tumors when there is a high risk for their development. A polymer according to the present invention may also be incorporated in access devices so that the radiosensitizer is released into the body cavity within which the access device resides, thereby preventing or treating the development of a neoplasm. The polymer composition of the invention may also be used to produce coatings for other solid implantable devices.

Once a system or implant article is in place, it should remain in at least partial contact with a biological fluid, such as blood, tissue fluid, fluid within body cavities, cerebrospinal fluid or secretions from organ surfaces or mucous membranes, and the like.

G. Methods of Use of Subject Compositions Encapsulating Radiosensitizers

Upon administration of a subject composition to a patient or some other appropriate use, additional procedures may typically be envisioned. For example, in many embodiments, radiosensitizers may be administered either during a surgical procedure or in conjunction with some other interventional procedure. Depending upon the site and nature of the neoplasm, the patient often is treated with radiotherapy. When directed to a malignant neoplasm, radiotherapy is intended to kill residual neoplastic cells, thus diminishing the probability of recurring tumor and improving patient outcome. When directed to a benign neoplasm or vascular malformation, radiotherapy is intended to control the neoplastic process without having to resort to actual surgical procedures. In either situation, radiosensitizers may increase the efficacy of radiotherapy in controlling neoplasms, or may permit a smaller dose to be administered, thereby diminishing the incidence and severity of radiation aftereffects.

For example, in certain instances when the disease being treated involves a solid tumor, one of the first treatment steps may be surgical removal of the tumor. Despite such removal, residual tumor cells remaining at the site of excision may proliferate, thereby forming a recurrent tumor at the site. In addition, the tumor may be infiltrative thereby making it difficult if not impossible to remove it surgically. The problem of incomplete resection is particularly significant in those anatomic areas where a classical en bloc resection would remove or damage vital structures, e.g., in the central nervous system or in the head and neck regions. Administering post-operative radiation is common in those cases where tumor resection is believed to be macroscopically or microscopically incomplete. The administration of radiosensitizer compositions according to the present invention may be performed at the time of the resectional surgery when the possibility of post-operative radiation becomes apparent, i.e., in those cases where the extent of the tumor appears greater than what can be surgically removed. Alternatively, the administration of radiosensitizer compositions may be performed in the early post-operative period after the pathology report has indicated incomplete resection, before the commencement of radiotherapy. In these situations, formulations of radiosensitizer compositions may be prepared that are adapted for instillation within the surgical site using any of the medical or surgical methods familiar to practitioners in these fields.

Furthermore, there may be locoregional spread of the disease, with involvement of the lymph node systems draining the tumor site. Administration of radiosensitizers, either directly at the time of surgery or as a separate procedure, for example during lymphangiography, may enhance the effectiveness of locoregional radiation in controlling the spread of the disease.

Physical properties of a subject composition encapsulating a radiosensitizer may be selected by skilled artisans based on the particular anatomy of the region to be treated. For example, a breast cancer patient who has undergone local tumor excision (e.g., “lumpectomy”) to be followed by radiation treatment may benefit from placement within the excision cavity of a polymeric substance in the form of a gel that can gradually infiltrate the tissues surrounding the excision cavity, the areas most likely to be affected by residual tumor. As another example, a patient with a gynecological malignancy that has spread within the pelvis or the peritoneal cavity may be a candidate for post-operative radiation, a treatment whose efficacy may be enhanced by the intraoperative administration of a radiosensitizer composition according to the present invention, delivered as a spray or liquid to be administered topically, or a gel to occupy dead space left behind by the extirpative surgery. If more durable filling of dead space is desirable following tumor extirpation, a more solid composition may be delivered, formed in a shape that allows it to occupy the dead space effectively and atraumatically. Such a composition may have the additional properties of holding open body lumens (i.e., stenting them) during the course of radiation treatment and subsequent tissue healing. Other formulations may be provided that can be administered at a distance from the target area, for example within a blood vessel, lymphatic or duct, with the intent that the substance flow or be carried to the target. In these cases, the target can thereafter be subjected to radiation.

As know to those of skill in the art, different combinations or radiotherapy may be used, such as HDR and ULDR, which have different effects on tissue. For example, ULDR is believed to reduce repair of damage to DNA, whereas HDR is believed to have different affects depending on the phase of growth of a tumor cell. In certain subject methods, the use of more than one type of radiation provides the most effective treatment when used in combination with the subject compositions.

If external beam radiotherapy is the source of electromagnetic radiation, radiotherapy treatment could begin after surgery. The subject biodegradable composition administered to the patient would release radiosensitizer and thus ideally improve the efficacy of radiotherapy. In longer courses of radiotherapy, the controlled release of radiosensitizer achievable in certain embodiments of the present invention would allow the combination of radiotherapy and treatment with the radiosensitizer to proceed. In certain cases, radiotherapy treatment by external beam radiation is administered to a patient in advance of surgery or as an alternative to surgery. In those cases where radiotherapy precedes surgery, it is desirable that the future operative site sustain as little radiation damage as possible. Radiosensitizers may increase the efficacy of the delivered radiation in controlling the neoplasm. Radiosensitizers furthermore may reduce the amount of radiation required to achieve the desired pre-operative reduction of tumor bulk.

Certain categories of neoplasms are treated with radiation alone, involving no surgery. In each category, cases may exist where administration of radiosensitizers according to the present invention may make radiotherapy more effective in standard doses, or sufficiently effective in reduced doses that radiation-related complications may be diminished. Some of these neoplasms treated with radiation alone are very small ones, especially in the head and neck region, where radiation is viewed as a cosmetically preferable treatment modality over surgery. Some of these tumors are very extensive, where surgery offers no chance of cure and where radiation is considered to be a less traumatic step towards palliation. For example, some advanced cancers of the lung, and the head and neck fall in this category. A third category of neoplasms treated with radiation alone are those located in anatomic regions with limited or difficult surgical access. In these cases, the surgical approach to the tumor and the surgical resection thereof are sufficiently dangerous or mutilating that the alternative of radiation is preferred.

Alternatively, instead of an external beam of electromagnetic radiation, radiotherapy-brachytherapy may be used, in which small radioactive seeds are placed at the neoplasm site (and elsewhere as appropriate). Subject compositions encapsulating a radiosensitizer would be administered either before or after the radioactive seeds were administered. The combination of the seeds and the polymer matrix would allow treatment by the radiosensitizer and electromagnetic radiation to proceed.

Sometimes tumors are physically located in parts of the body that make surgical removal difficult or impossible. Non-limiting examples would include tumors in deep-seated areas of the brain, mediastinal tumors proximal to the aorta and/or the heart, and neck tumors proximal to the carotid artery. In these and other situations, it is medically desirable to administer the composition containing the radiosensitizer in a non-invasive manner.

In certain embodiments, interventional medicine may be used to deliver a subject composition encapsulating a radiosensitizer to deep-seated areas of the body with the help of radiological imaging. Upon imaging the body site of interest using technology such as x-ray, fluoroscopy, ultrasound, computer aided tomograph scans, and MRI, a needle, a catheter, a trochar or any other device may be used to access the site of interest and administer the subject polymer at the site. Subsequent treatment with electromagnetic radiation would utilize the therapeutic properties of any radiosensitizer released from the subject matrix.

In addition to the embodiments described above related to treatment of neoplasm, other non-limiting uses for the present invention may be contemplated. For example, recognizing the beneficial effects of local radiation in controlling restenosis after angioplasty, local delivery of radiosensitizing compositions to the vessel wall may be performed in conjunction with an angioplasty procedure, followed by the delivery of electromagnetic radiation thereto. Or, for example, microspheres may be delivered angiographically to a site of an arteriovenous malformation or to an area of tumor neovascularization bearing the radiosensitizing compositions of the present invention, to be followed by an appropriate dose of electromagnetic radiation. Other uses for the inventive compositions and methods in treating benign and malignant diseases will be readily envisioned by practitioners in the relevant arts, using no more than routine experimentation.

The use of the subject invention has been shown to provide more effective therapies than treatment with electromagnetic radiation alone (see the appended Examples). In those examples, the increase in the mass of the implanted tumor was delayed to a greater extent upon treatment with the subject compositions in conjunction with electromagnetic radiation as compared to the use of the same course of electromagnetic radiation alone. Also, in certain embodiments, the tumor no longer increased in volume or even decreased upon use of the subject compositions in conjunction with electromagnetic radiation. Alternatively, treatment with the subject compositions in connection with electromagnetic radiation treatment resulted in improved growth inhibition. For example, it may be the case that the rate of increase in volume of tumor upon use of the subject composition in conjunction with electromagnetic radiation is three-quarters, two-thirds, one-half, one-third, one-quarter, one-fifth or even less than the rate of increase of the tumor volume for treatment with electromagnetic radiation without administering the subject composition. Also, in certain embodiments, it may take the tumor fifty percent, seventy-five percent, one hundred percent, two hundred percent or even more time (if ever) for the tumor volume observed upon treatment with the combined therapy to reach that observed for the tumor volume resulting from treatment with electromagnetic radiation alone.

In certain embodiments, use of the subject compositions encapsulating a radiosensitizer in conjunction with electromagnetic radiation should result in a therapeutic index of the electromagnetic radiation greater than that that obtained upon treatment of said patient with electromagnetic radiation without administering said composition. In certain of such embodiments, the therapeutic index of the combined therapy should be three, five ten, fifty, one hundred or even larger multiples greater than that obtained for treatment with radiation alone.

Likewise, remission of a disease or condition, often a neoplasm, may be more likely, a lower dose of radiation may be used to achieve remission, and remission periods may be longer using the subject invention in conjunction with electromagnetic radiation as compared to the use of electromagnetic radiation alone. For example, in certain embodiments, it may be the case that the dose of radiation necessary to achieve remission using the subject compositions will be three-quarters, two-thirds, one half, a third, a quarter or even less that that required to achieve remission without the use of such compositions. Also, it may be the case that longer remission times are achieved, such as 25%, 50%, 75%, two, three or even longer times, using the subject compositions in conjunction with electromagnetic radiation as compared to the use of such radiation alone.

Also, the survival times of patients treated with the combined therapy using the subject invention may be greater than those observed for patients treated with electromagnetic radiation alone. In certain embodiments, the survival rates for patients treated with the combined therapy may be greater by a factor of 50%, 100%, 200% or even five and ten times those obtained for patients treated with electromagnetic radiation alone.

In considering the effectiveness of the subject invention, in vitro and in vivo models, like those described in the appended Examples, and others known to those of skill in the art, may be used in certain instances to gauge the effectiveness of the subject invention in accordance with the metrics described above.

H. Exemplary Methods of Making Subject Compositions

In general, the polymers of the present invention may be prepared by melt polycondensation, solution polymerization or interfacial polycondensation. Techniques necessary to prepare the subject polymers are known in the art, and reference is made in particular to U.S. Provisional Application Serial No. 60/216,462 filed Jul. 6, 2000 and U.S. Provisional Application Serial No. 60/228,729 filed Aug. 29, 2000, both of which are hereby incorporated in their entirety.

The most common general reaction in preparing the subject compositions is a dehydrochlorination between a phosphodichloridate and a diol according to the following equation:

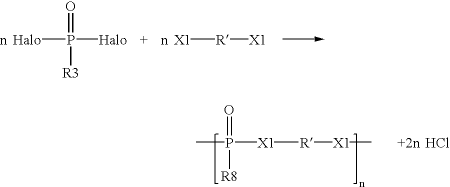

Certain of the subject polymers may be obtained by condensation between appropriately substituted dichlorides and diols.

An advantage of melt polycondensation is that it avoids the use of solvents and large amounts of other additives, thus making purification more straightforward. This method may also provide polymers of reasonably high molecular weight. Somewhat rigorous conditions, however, are often required and may lead to chain acidolysis (or hydrolysis if water is present). Unwanted, thermally-induced side reactions, such as cross-linking reactions, may also occur if the polymer backbone is susceptible to hydrogen atom abstraction or oxidation with subsequent macroradical recombination.

To minimize these side reactions, the polymerization may also be carried out in solution. Solution polycondensation requires that both the prepolymer and the phosphorus component be sufficiently soluble in a common solvent. Typically, a chlorinated organic solvent is used, such as chloroform, dichloromethane or dichloroethane. The solution polymerization is generally run in the presence of equimolar amounts of the reactants and, preferably, an excess of an acid acceptor and a catalyst, such as 4-dimethylaminopyridine (DMAP). Useful acid acceptors include tertiary amines as pyridine or triethylamine. The product is then typically isolated from the solution by precipitation in a non-solvent and purified to remove the hydrochloride salt by conventional techniques known to those of ordinary skill in the art, such as by washing with an aqueous acidic solution, e.g., dilute HCl.

Reaction times tend to be longer with solution polymerization than with melt polymerization. However, because overall milder reaction conditions may be used, side reactions are minimized, and more sensitive functional groups may be incorporated into the polymer. The disadvantages of solution polymerization are that removal of solvents may be difficult.

Interfacial polycondensation may be used when high molecular-weight polymers are desired at high reaction rates. By such methods, mild conditions minimize side reactions, and the dependence of high molecular weight on stoichiometric equivalence between diol and dichloridate inherent in solution methods is removed. However, hydrolysis of the acid chloride may occur in the alkaline aqueous phase, and sensitive dichloridates that have some solubility in water are generally subject to hydrolysis rather than polymerization. Phase transfer catalysts, such as crown ethers or tertiary ammonium chloride, may be used to bring the ionized diol to the interface to facilitate the polycondensation reaction. The yield and molecular weight of the resulting polymer after interfacial polycondensation are affected by reaction time, molar ratio of the monomers, volume ratio of the immiscible solvents, the type of acid acceptor, and the type and concentration of the chase transfer catalyst.

Methods for making the present invention may take place at widely varying temperatures, depending upon whether a solvent is used and, if so, which one; the molecular weight desired; the susceptibility of the reactants to form side reactions; and the presence of a catalyst. Usually, the process takes place at a temperature ranging from about 0 to about 235° C. for melt conditions. Somewhat lower temperatures, e.g., for example from about −50 to about 100° C., may be possible with solution polymerization or interfacial polycondensation with the use of either a cationic or anionic catalyst.

The time required for the process may vary widely, depending on the type of reaction being used, the molecular weight desired and, in general, the need to use more or less rigorous conditions for the reaction to proceed to the desired degree of completion. Typically, however, the synthetic process takes place during a time between about 30 minutes and about 7 days.

Although the process may be in bulk, in solution, by interfacial polycondensation, or any other convenient method of polymerization, in many instant embodiments, the process takes place under solution conditions. Particularly useful solvents include methylene chloride, chloroform, tetrahydrofuran, di-methyl formamide, dimethyl sulfoxide or any of a wide variety of inert organic solvents.

In greater detail, polymers of Formula VI may be prepared, at least in part, by reacting a compound having a formula H-Y1-L1-Y1-H, such as 2-aminoethanol, ethylene glycol, ethane dithiol, etc., with a cyclic compound, e.g., having one of the following structures: for example, caprolactone or lactide (lactic acid dimer).

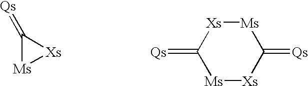

Thus, the cyclic compound may include one or two subunits ts. For cyclic compounds containing two subunits, the two subunits contained therein may be the same or different.

For synthesizing, for example, a compound of Formula VI, wherein x and y are on average about 10, an equivalent of ethylene glycol as H-Y1-L1-Y1-H may be reacted with 20 equivalents of

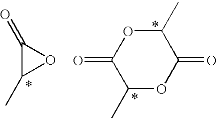

because lactic acid dimer contains two monomer units for each equivalent of the cyclic compound. Variation of the ratio of cyclic compound to ethylene glycol or other bifunctional core will likewise vary the values of x and y, although x and y will be substantially equal for a symmetrical bifunctional core (e.g., ethylene glycol) for subject polymers prepared by this method. For an unsymmetrical bifunctional core (e.g., 2-aminoethanol), the ratio of x:y may vary considerably, as will be understood by one of skill in the art and may be determined without undue experimentation.

Polymers of the present invention may generally be isolated from the reaction mixture by conventional techniques, such as by precipitating out, extraction with an immiscible solvent, evaporation, filtration, crystallization and the like. Typically, the subject polymers are both isolated and purified by quenching a solution of polymer with a non-solvent or a partial solvent, such as diethyl ether or petroleum ether.

In certain embodiments, the subject polymers are soluble in one or more common organic solvents for ease of fabrication and processing. Common organic solvents include such solvents as chloroform, dichloromethane, dichloroethane, 2-butanone, butyl acetate, ethyl butyrate, acetone, ethyl acetate, dimethylacetamide, N-methyl pyrrolidone, dimethylformamide, and dimethylsulfoxide.

I. Dosages and Formulations

In most embodiments, the subject polymers will incorporate the substance to be delivered in an amount sufficient to deliver to a patient a therapeutically effective amount of an incorporated therapeutic agent or other material as part of a prophylactic or therapeutic treatment. The desired concentration of active compound in the particle will depend on absorption, inactivation, and excretion rates of the drug as well as the delivery rate of the compound from the matrix, as well as the use of electromagnetic radiation in conjunction with the subject compositions. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

The present invention allows a significant percentage of any radiosensitizer to be administered to a patient in one administration of a subject composition as opposed to admininstration of smaller doses of the radiosensitizer more frequently, e.g., before each treatment with electromagnetic radiation as would be the case for other forms of administration of a radiosensitizer not so encapsulated. For example, 25%, 50%, 75% even substantially all of the dosage of a radiosensitizer may be administered once to a patient contemplating a course of electromagnetic radiation. Alternatively, the subject polymer matrices may be divided into a number of smaller doses to be administered at varying intervals of time as appropriate.

In certain embodiments, the subject compositions comprise about 5% to about 60%, alternatively about 10% to about 50% of an radiosensitizer, such as IUdR, in a biodegradable polymer, such as a phosphorous-based polymer, e.g., P(trans-CHDM/HOP). In certain embodiments, a composition comprises at least about 10% of an radiosensitizer, more particularly at least about 20%, at least about 25%, 30%, 40%, 50% or even more than about 50% of a radiosensitizer.

The polymers of the present invention may be administered by various means, depending on its intended use, as is well known in the art, some of which have been described above. In addition, the subject compositions may be formulated using techniques and methods known to those of skill in the art. In addition, in certain embodiments, polymer matrices of the present invention may be lyophilized.

### EXEMPLIFICATION

The invention now being generally described, it will be more readily understood by reference to the following examples which are included merely for purposes of illustration of certain aspects and embodiments of the present invention, and are not intended to limit the invention.

### Example 1

### First Synthesis of D,L-PL(PG)EOP

All glassware was dried for a minimum of 2 hours at 105° C. and allowed to cool in a desiccator or cooled under a stream of argon gas. A 28.5 g portion of D,L-lactide and 1.5 g of 1,2-propanediol (PG), obtained from Aldrich, Catalog No. 39,803, 99.5+%, in a molar ratio of 10:1, were weighed into a 250 mL 3-neck round-bottom flask. The flask was equipped with a gas joint and a stirrer bearing/shaft/paddle assembly. The mixture was evacuated and pressurized with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution (about 130 mg/ml in toluene of chloroform) equivalent to 3.6 mg tin (120 ppm stannous octoate or equivalent to 35 ppm tin based upon weight of the prepolymer) was added to the melt using a 50 μl syringe. The reaction mixture was allowed to stir under a slight argon pressure for approximately 16 hours. The oil bath temperature was then reduced to about 110° C. and the residual monomer was removed under vacuum. The upper parts of the reaction assembly were heated gently with a heat gun to aid in the monomer removal. The total time under vacuum was 2-3 hours. A reflux condenser was then inserted between the gas joint and the flask in the prepolymer apparatus described above. The molten prepolymer was dissolved by adding 100 mL of chloroform to the reaction flask with stirring.

Next, 6.9 mL of triethylamine (TEA) and 1.21 g of DMAP were added to the stirring reaction mixture. The reaction mixture was then chilled to about 4° C. in an ice bath. A solution of approximately 2.5 mL of freshly distilled ethyl dichlorophosphate (EOPCl₂) in 25 mL of chloroform was prepared in a dropping funnel. The solution in the funnel was added drop wise to the reaction mixture over a period of about 30 minutes. After the addition was complete the reaction mixture was allowed to continue stirring at about 4° C. for 10 minutes and then the ice bath was removed. The reaction mixture was allowed to warm to room temperature over about 1 hour. At this time a significant increase in viscosity of the clear solution was observed. The reaction mixture was then heated to reflux using an oil bath. Over the next hour the solution became cloudy. The reaction mixture was allowed to reflux over two nights, about 38 hours total.

At this time, a Barret trap was inserted between the condenser and the flask and 88 mL of solvent (2/3 of the total volume) were distilled from the reaction mixture. The Barret trap was removed and the reaction mixture was allowed to reflux for an additional 16 hours with the oil bath temperature between 98-102° C. Next, the oil bath temperature was increased to 115° C. for 2 hours. After this time, the reaction mixture was allowed to cool to room temperature, and 200 mL of dichloromethane was added and transferred to a separatory funnel. The reaction mixture was extracted twice with 100 mL of 0.1 M HCl and twice with 100 mL of saturated sodium chloride solution. The organic layer was isolated, dried overnight in the freezer at about −15° C. over 50 g of sodium sulfate, and filtered twice. The resulting polymer solution was poured into 1500 mL of hexane plus 500 mL of ether. The resulting mass of polymer was dried under vacuum. The Inherent Viscosity (IV) of this material was measured to be 0.39 dL/g.

### Example 2

### Second Synthesis of D,L-PL(PG)EOP

All glassware was dried for a minimum of 2 hours at 105° C. and allowed to cool in a desiccator or cooled under a stream of argon gas. A 28.5 g portion of D,L-lactide and 1.5 g of PG (molar ratio, 10:1) were weighed into a 250 ml 3-neck round bottom flask. The flask was equipped with a gas joint and a stirrer bearing/shaft/paddle assembly. The mixture was evacuated and filled with argon five times to remove residual air and moisture. Each time the polymerization vessel was evacuated to a pressure between 0.5 and 10 Torr. The reaction apparatus was immersed in a preheated oil bath at 125° C., connected to an argon source with an oil bubbler, and stirred at a moderate speed until all of the solid monomer had melted. At this time, a volume of stock stannous octoate solution (about 130 mg/ml in toluene) equivalent to 100 ppm stannous octoate (29 ppm Sn) was added to the melt using a syringe. The reaction mixture was allowed to stir under a slight argon pressure for 3 hours. The oil bath temperature was then reduced to about 105° C. and the residual monomer was removed under vacuum. The pressure was maintained as low as possible, typically between 0.5 and 10 Torr. The upper parts of the reaction assembly were heated gently with a heat gun to aid in the monomer removal. The total time under vacuum was 1 hour.

The prepolymer was cooled to room temperature under argon gas and allowed to stand for 12-18 hours at ambient temperature. The prepolymer was dissolved in 84 ml of chloroform with stirring and 2.5 equivalents of TEA and 0.5 equivalents of DMAP were added to the stirring reaction mixture using a powder funnel. The reaction mixture was chilled to about −5° C. to about −15° C. in a cold bath. A solution of about 1 equivalent of distilled ethyl dichlorophosphate (EOPCl₂) in 10 ml of chloroform was prepared in a dropping funnel. The solution in the funnel was added slowly to the reaction mixture over a period of 0.5 hour.

After the addition was complete, the reaction mixture was allowed to stir at low temperature for 1 hour at −5° C. The reaction was then quenched with 1 ml of anhydrous methanol and stirred for another five minutes. Next, the reaction mixture was transferred to a 0.5 gallon vessel and mixed with 37 g of Dowex DR-2030 IER and 30 g of Dowex M-43, and shaken on a mechanical shaker for 2 hour to remove residual DMAP and TEA free base and salts (the IERs had been washed with several bed volumes of methanol and chloroform and dried under vacuum at ambient temperature for about 18 hours). The resin was removed from the reaction mixture by vacuum filtration through Whatman 54 filter paper.

The resin was washed with about one bed volume of dichloromethane and the filtrate was concentrated to approximately 50 ml. The viscous filtrate was poured into 200 ml of petroleum ether to precipitate the polymer. The polymer mass was washed with 100 ml of petroleum ether and dried under vacuum. Molecular weights of the polymers were obtained from gel permeation chromatography (GPC) using both differential refractive index detection and a polystyrene calibration curve (CC) and by light scattering detection. The molecular weight and IV data for the polymers prepared by this process are listed in the table below.

| Sample | Mw (LS), daltons | Mw (CC), daltons | IV, dL/g |
|---|---|---|---|
| 1 | 101,200 | 107,500 | 0.62 |
| 2 | 150,100 | 155,900 | 0.80 |
| 3 | 85,200 | 84,300 | — |
| 4 | 92,600 | 89,900 | — |

### Example 3

### Synthesis of D,L-PL(EG)EOP

All glassware was dried for a minimum of 2 hours at 105° C. and allowed to cool in a desiccator or cooled under a stream of argon gas. A 100.0 g portion of D,L-lactide and 4.3 g of ethylene glycol (EG) (molar ratio, 10:1) were weighed into a 1000 ml 3-neck round bottom flask. The flask was equipped with a gas joint and a stirrer bearing/shaft/paddle assembly. The mixture was evacuated and filled with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution (about 130 mg/ml in toluene) equivalent to 120 ppm stannous octoate or 35 ppm Sn was added to the melt using a syringe. The reaction mixture was allowed to stir under a slight argon pressure for approximately 16 hours. The oil bath temperature was then reduced to about 110° C. and the residual monomer was removed under vacuum. The upper parts of the reaction assembly were heated gently with a heat gun to aid in the monomer removal. The total time under vacuum was 2-3 hours.

The molten prepolymer was dissolved in 350 ml of chloroform with stirring and 2.5 equivalents of TEA and 0.5 equivalents of DMAP were added to the stirring reaction mixture using a powder funnel. The reaction mixture was chilled to about −5° C. in a cold bath. A solution of about 1 equivalent of distilled ethyl dichlorophosphate (EOPCl₂) in 97 ml of chloroform was prepared in a dropping funnel. The solution in the funnel was added slowly to the reaction mixture over a period of 2 hours. After the addition was complete, the reaction mixture was allowed to stir at low temperature for 45 minutes at −5° C. After 2 hours a significant increase in viscosity of the clear solution was observed. The reaction was then quenched with 6.8 ml of anhydrous methanol and stirred for another five minutes.

Next, the reaction mixture was transferred to a 0.5 gallon vessel and mixed with 87 g of Dowex HCR-S IER and 104 g of Dowex-43, and shaken on a mechanical shaker for 1 hour to remove residual DMAP and TEA free base and salts (the IERs had been washed with several bed volumes of methanol and dried under vacuum at ambient temperature for about 18 hours). The resin was removed from the reaction mixture by vacuum filtration through Whatman 54 filter paper. The resin was washed with about one bed volume of dichloromethane and the filtrate was concentrated to approximately 150 ml. The viscous filtrate was poured into 2000 ml of hexane to precipitate the polymer. The polymer mass was washed with 2×200 ml of hexane and dried under vacuum. The molecular weights were determined by GPC were 40,400 for Mw (LS) and 42,000 for Mw (CC).

### Example 4

### Synthesis of D,L-PL(HD)EOP

All glassware was dried for a minimum of 2 hours at 105° C. and allowed to cool in a desiccator or cooled under a stream of argon gas. A 100.0 g portion of D,L-lactide and 8.2 g of 1,6 hexane diol (HD) (molar ratio, 10:1) were weighed into a 1000 ml 3-neck round bottom flask. The flask was equipped with a gas joint and a stirrer bearing/shaft/paddle assembly. The mixture was evacuated and filled with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution equivalent (about 130 mg/ml in toluene) to 120 ppm stannous octoate or 35 ppm Sn was added to the melt using a syringe. The reaction mixture was allowed to stir under a slight argon pressure for approximately 16 hours. The oil bath temperature was then reduced to about 110° C. and the residual monomer was removed under vacuum. The upper parts of the reaction assembly were heated gently with a heat gun to aid in the monomer removal. The total time under vacuum was 2-3 hours.

The molten prepolymer was dissolved in 350 ml of chloroform with stirring and 2.5 equivalents of TEA and 0.5 equivalents of DMAP were added to the stirring reaction mixture using a powder funnel. The reaction mixture was chilled to about −5° C. in a cold bath. A solution of about 1 equivalent of distilled ethyl dichlorophosphate (EOPCl₂) in 97 ml of chloroform was prepared in a dropping funnel. The solution in the funnel was added slowly to the reaction mixture over a period of 2 hours. After the addition was complete, the reaction mixture was allowed to stir at low temperature for 45 minutes at −5° C. After 2 hours a significant increase in viscosity of the clear solution was observed. The reaction was then quenched with 6.8 ml of anhydrous methanol and stirred for another five minutes.

Next, the reaction mixture was transferred to a 0.5 gallon vessel and mixed with 87 g of Dowex HCR-S IER and 104 g of Dowex-43, and shaken on a mechanical shaker for 1 hour to remove residual DMAP and TEA free base and salts (the IERs had been washed with several bed volumes of methanol and dried under vacuum at ambient temperature for about 18 hours). The resin was removed from the reaction mixture by vacuum filtration through Whatman 54 filter paper. The resin was washed with about one bed volume of dichloromethane and the filtrate was concentrated to approximately 150 ml. The viscous filtrate was poured into 2000 ml of hexane to precipitate the polymer. The polymer mass was washed with 2×200 ml of hexane and dried under vacuum. The molecular weights were determined by GPC were 36,700 for Mw (LS) and 34,100 for Mw (CC). The value for IV was 0.33 dL/g.

### Example 5

### Polymer of PG, D,L-lactide, Glycolide, and Ethyl Dichlorophosphate

All glassware was dried for a minimum of 2 hours at 105° C. and allowed to cool in a desiccator or cooled under a stream of argon gas. A 28.5 g portion of D,L-lactide and 1.5 g of PG (molar ratio, 10:1) were weighed into a 250 ml 3-neck round bottom flask. The flask was equipped with a gas joint and a stirrer bearing/shaft/paddle assembly and a 125 ml dropping funnel containing 4.6 g of glycolide. The mixture was evacuated and filled with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution (about 130 mg/ml in toluene) equivalent to 3.6 mg tin (120 ppm stannous octoate or 35 ppm tin) was added to the melt using a 50 μl syringe. The reaction mixture was allowed to stir under a slight argon pressure for approximately 16 hours. At this time the glycolide was melted using a heat gun and added to the polymer melt in the flask. The melt was stirred for an additional 2 hours. The oil bath temperature was then reduced to about 115° C. and the residual monomer was removed under vacuum. The upper parts of the reaction assembly were heated gently with a heat gun to aid in the monomer removal. The total time under vacuum was 2 hours.

The molten prepolymer was suspended in 84 ml of chloroform with stirring and 2.5 equivalents of TEA and 0.5 equivalents of DMAP were added to the stirring reaction mixture using a powder funnel. The reaction mixture was chilled to about 4° C. in a cold bath. A solution of about 1 equivalent of distilled ethyl dichlorophosphate (EOPCl₂) in 27.5 ml of chloroform was prepared in a dropping funnel. The solution in the funnel was added slowly to the reaction mixture over a period of 1 hour. After the addition was complete, the reaction mixture was allowed to stir at low temperature for another 1.75 hours and then the cold bath was removed. The reaction mixture was allowed to warm to room temperature and stirred for 2 to 18 hours. After 2 hours a significant increase in viscosity of the clear solution was observed. The reaction was then quenched with 1 ml of anhydrous methanol and stirred for another five minutes.

Next, 37 g of dry Dowex HCR-S IER and 30 g of dry Dowex M-43 were added to the reaction mixture and stirring was continued for another hour to remove residual DMAP and TEA free base and salts. The IERs were removed from the reaction mixture by vacuum filtration through Whatman 54 filter paper. The resin was washed with about one bed volume of dichloromethane and the filtrate was concentrated to approximately 50 ml. The viscous filtrate was poured into 700 ml of petroleum ether to precipitate the polymer and dried under vacuum.

### Example 6

### Synthesis of D,L-PL(PG)HOP

All glassware was dried for a minimum of 2 hours at 105° C. and allowed to cool in a desiccator or cooled under a stream of argon gas. A 28.5 g portion of D,L-lactide and 1.5 g of PG (molar ratio, 10:1) were weighed into a 250 ml 3-neck round bottom flask. The flask was equipped with a gas joint and a stirrer bearing/shaft/paddle assembly. The mixture was evacuated and filled with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution (about 130 mg/ml in toluene) equivalent to 3.6 mg tin (120 ppm stannous octoate or 35 ppm tin) was added to the melt using a 50 μl syringe. The reaction mixture was allowed to stir under a slight argon pressure for approximately 16 hours. The oil bath temperature was then reduced to about 110° C. and the residual monomer was removed under vacuum. The upper parts of the reaction assembly were heated gently with a heat gun to aid in the monomer removal. The total time under vacuum was 2-3 hours.

The molten prepolymer was dissolved in 100 ml of chloroform with stirring and TEA and DMAP were added to the stirring reaction mixture using a powder funnel. The funnel was rinsed with 10 ml of chloroform. The reaction mixture was chilled to about 4° C. in a cold bath. A solution of about 1 equivalent of distilled hexyl dichlorophosphate (HOPCl₂) in 27.5 ml of chloroform was prepared in a dropping funnel. The solution in the funnel was added slowly to the reaction mixture over a period of 1 hour. After the addition was complete, the reaction mixture was allowed to stir at low temperature for another hour and then the cold bath was removed. The reaction mixture was allowed to warm to room temperature and stirred for 2 to 18 hours. After 2 hours a significant increase in viscosity of the clear solution was observed. The reaction was then quenched with 800 μl of anhydrous methanol and stirred for another five minutes.

Next, Dowex MR-3C ion exchange resin (IER) was added to the reaction mixture and stirring was continued for another hour to remove residual DMAP and TEA free base and salts (the Dowex resin had been washed with several bed volumes of methanol and dried under vacuum at ambient temperature for about 18 hours). The resin was removed from the reaction mixture by vacuum filtration through Whatman 54 filter paper. The resin was washed with about one bed volume of dichloromethane and the filtrate was concentrated to approximately 100 ml. The viscous filtrate (now a somewhat cloudy solution) was poured into 1000 ml of hexane to precipitate the polymer. The polymer mass was washed with 2×200 ml of hexane and dried under vacuum. The molecular weight and IV data for the polymers prepared by this process are listed in the table below.

| Sample | Mw (LS), daltons | Mw (CC), daltons | IV, dL/g |
|---|---|---|---|
| 1 | 64,200 | 58,000 | 0.48 |
| 2 | 68,000 | 62,700 | 0.43 |

### Example 7

### Synthesis of D,L-PL(PG)EP

All glassware was dried for a minimum of 2 hours at 105° C. and allowed to cool in a desiccator or cooled under a stream of argon gas. A 28.5 g portion of D,L-lactide and 1.5 g of PG (molar ratio, 10:1) were weighed into a 250 ml 3-neck round bottom flask. The flask was equipped with a gas joint and a stirrer bearing/shaft/paddle assembly. The mixture was evacuated and filled with argon five times to remove residual air and moisture. The reaction apparatus was immersed in a preheated oil bath at 130° C., connected to an argon source with an oil bubbler, and stirred at a moderate speed until all of the solid monomer had melted.

At this time, a volume of stock stannous octoate solution (about 130 mg/ml in toluene) equivalent to 120 ppm stannous octoate or 35 ppm Sn was added to the melt using a syringe. The reaction mixture was allowed to stir under a slight argon pressure for 4 hours. The oil bath temperature was then reduced to about 110° C. and the residual monomer was removed under vacuum. The upper parts of the reaction assembly were heated gently with a heat gun to aid in the monomer removal. The total time under vacuum was 2 hours.

The molten prepolymer was dissolved in 84 ml of chloroform with stirring and 2.5 equivalents of TEA and 0.5 equivalents of DMAP were added to the stirring reaction mixture using a powder funnel. The reaction mixture was chilled to about −5° C. in a cold bath. A solution of about 1 equivalent of distilled ethyl dichlorophosphonate (EPCl₂) in 9 ml of chloroform was prepared in a dropping funnel. The solution in the funnel was added slowly to the reaction mixture over a period of 0.5 hour. After the addition was complete, the viscosity of the solution had increased significantly and the reaction mixture was allowed to stir at low temperature for 1 hour at −5° C. The reaction was then quenched with 1 ml of anhydrous methanol and stirred for another five minutes.

Next, the reaction mixture was transferred to a 0.5 gallon vessel and mixed with 37 g of Dowex DR-2030 IER and 30 g of Dowex-43, and shaken on a mechanical shaker for 2 hour to remove residual DMAP and TEA free base and salts (the IERs had been washed with several bed volumes of methanol and chloroform and dried under vacuum at ambient temperature for about 18 hours). The resin was removed from the reaction mixture by vacuum filtration through Whatman 54 filter paper. The resin was washed with about one bed volume of dichloromethane and the filtrate was concentrated to approximately 50 ml. The viscous filtrate was poured into 200 ml of petroleum ether to precipitate the polymer. The polymer mass was washed with 100 ml of petroleum ether and dried under vacuum. The molecular weight data for the polymers prepared by this process are listed in the table below.

| Sample | Mw (LS), daltons | Mw (CC), Daltons |
|---|---|---|
| 1 | 339,900 | 327,600 |
| 2 | 369,800 | 360,900 |

### Example 8

### Synthesis of P(cis- and trans-CHDM/HOP)

All glassware was dried for a minimum of two hours at 105° C. and allowed to cool in a desiccator or cooled under a stream of argon gas. A reaction assembly consisting of a 1 L three neck round bottom flask equipped with a gas joint, a stirrer bearing/shaft/paddle and a dropping funnel. A solution of 20.0 g of 1,4-cyclohexane dimethanol (CHDM) was prepared in 75 ml of anhydrous tetrahydrofuran (THF) and transferred to the reaction vessel. The beaker was rinsed with 25 ml of THF and the wash was transferred to the reaction vessel.

Next, 29.0 ml of N-methylmorpholine (NMM) and 1.61 g of DMAP were added to the reaction mixture through a powder funnel. A solution of 28.86 g of hexyl dichlorophosphate (HOPCl₂) in 30 ml of THF was prepared under argon and transferred to the dropping funnel while the reaction mixture was cooled to 4° C. in a cold bath. The solution in the funnel was added to the reaction mixture over a period of one hour. With 5 to 10 minutes after the start of addition, a white precipitate, presumably the hydrochloride salts of NMM and DMAP, began to form. After the addition was complete the funnel was rinsed with 30 ml of THF. The reaction mixture was stirred for 1 hour at 4° C. and then for either 2 or 18 hours at ambient temperature.

At the prescribed time, the precipitate was removed from reaction mixture by vacuum filtration. The filtrate was diluted with 100 ml of dichloromethane, transferred to a half-gallon jar and 86.5 of dried Dowex HCR-S IER and 103.8 g of dried Dowex M-43 IER were added to the filtrate. The jar was sealed with a Teflon lined lid and the mixture was agitated on a mechanical shaker for two hours.

At this time, the IERs were removed by vacuum filtration and the filtrate was concentrated to approximately 100 ml under vacuum. The polymer solution was poured in 2 L of hexane and the resulting fluid material that precipitated was isolated and transferred to a Teflon lined glass dish. The polymer was dried under vacuum to yield a sticky, free flowing viscous liquid. The Mw (LS) data for the polymers prepared by this process are listed in the table below.

| Sample | Mw (LS), daltons | Mw (CC), daltons | IV, dL/g |
|---|---|---|---|
| 1 | 4400 | 5500 | 0.14 |
| 2 | 5000 | 6500 | 0.11 |
| 3 | 4000 | 4600 | 0.10 |

The same method may be used to prepare the trans-CHDM version of the polymer, P(trans-CHDM/HOP).

### Example 9

### Synthesis of P(BHET/EOP)

All glassware was dried for a minimum of two hours at 105° C. and allowed to cool in a desiccator or cooled under a stream of argon gas. A reaction assembly consisting of a 500 ml three neck round bottom flask equipped with a gas joint, a stirrer bearing/shaft/paddle and a dropping funnel. First, 30.0 g of bis(hydroxyethyl) terephthalate (BHET) and 28.83 g of DMAP were added to the reaction vessel using a powder funnel and mixed with 81 ml of THF. The solids were dissolved with stirring and gentle heating using a heat gun.

After all solids had dissolved, the reaction mixture was cooled to 4° C. in a cold bath. A solution of 19.2 g of ethyl dichlorophosphate (EOPCl₂) in 24 ml of THF was prepared in a 125 ml addition funnel. The solution in the funnel was added to the solution in the flask over a period of 1 hour. Shortly after the addition had begun, a white precipitate, presumably DMAP hydrochloride, began to precipitate from the reaction mixture. After all of the solution in the funnel had been added, the stirrer shaft/paddle became entrapped in a thick, stiff precipitate and stirring ceased. It appears the polymer that had formed at this time was insoluble in the reaction mixture.

Next, 125 ml of dichloromethane were added and the reaction mixture was swirled by hand until mechanical stirring could be resumed. The reaction mixture was now a homogenous solution containing a white free flowing powder. The reaction mixture was stirred at 4° C. for one hour. The cold bath was removed and the reaction mixture was allowed to warm to ambient temperature and stirred for 16 hours. At this time, the white precipitate was removed from the reaction mixture by vacuum filtration and the filter cake was washed with 100 ml of dichloromethane.

The resulting filtrate was transferred to a half-gallon jar and treated with 156.92 g of undried Dowex HCR-S IER and 160.92 g of undried Dowex M-43 IER. The resins were washed with 2 bed volumes of methanol and 2 bed volumes of dichloromethane prior to use. The jar was sealed with a Teflon lined lid and shaken on a mechanical shaker for two hours. The resin was removed by vacuum filtration and the filtrate, ˜600 ml, was concentrated to ˜150 ml. The clear solution was poured into 1.2 L of hexane. The thick oil that precipitated was washed with 400 ml of hexane and transferred to a Teflon lined glass dish, dried under vacuum. The molecular weights were determined by GPC were 2200 for Mw (LS) and 2100 for Mw (CC). The value obtained for IV was 0.10 dL/g.

### Example 10

### Synthesis of P(BHET-EOP/TC)

All glassware was dried for a minimum of two hours at 105° C. and allowed to cool in a desiccator or cooled under a stream of argon gas. A reaction assembly consisting of a 500 ml three neck round bottom flask equipped with a gas joint, a stirrer bearing/shaft/paddle and a dropping funnel. First, 30.0 g of BHET and 28.83 g of DMAP were added to the reaction vessel using a powder funnel and mixed with 81 ml of THF and 125 ml of dichloromethane.

The solids were dissolved with stirring and gentle heating using a heat gun. After all solids had dissolved, the reaction mixture was cooled to 4° C. in a cold bath. A solution of 19.2 g of EOPCl₂ in 24 ml of THF was prepared in a 125 ml addition funnel. The solution in the funnel was added to the solution in the flask over a period of 1 hour. Shortly after the addition had begun, a white precipitate, presumably DMAP hydrochloride, began to precipitate from the reaction mixture. The reaction mixture was stirred at 4° C. for one hour. Next, a solution of 4.79 g of terephthaloyl chloride (TC) in 18 ml of THF was prepared in the addition funnel and added to the solution in the flask over a 30-minute period. The reaction mixture was stirred for one hour at 4° C.

At this time the cold bath was removed and the reaction was allowed to warm to room temperature and stir for another 20 hours. At this time, the white precipitate was removed from the reaction mixture by vacuum filtration. The resulting filtrate was transferred to a half-gallon jar and treated with 88.5 g of dried Dowex HCR-S IER and 73.8 g of dried Dowex M-43 IER. The jar was sealed with a Teflon lined lid and shaken on a mechanical shaker for two hours. The resin was removed by vacuum filtration and the filtrate was concentrated to ˜100 ml. The clear solution was poured into 2 L of hexane. The thick oil that precipitated was transferred to a Teflon lined glass dish, dried under vacuum. The molecular weights were determined by GPC were 7200 for Mw (LS) and 4000 for Mw (CC). The value obtained for IV was 0.09 dL/g.

### Example 11

### Large-Scale Preparation of D,L-PL(PG)EOP

A 100 g portion of propylene glycol was added to a 3000 ml 3-necked round bottom flask equipped with a gas joint, a stirrer bearing/shaft/paddle assembly, and a Teflon-coated thermocouple. The reaction apparatus was placed in a preheated oil bath at 130° C. and purged with nitrogen for one minute. A 2000 g portion of D,L-lactide was added using a powder addition funnel over a period of 45 minutes. The reaction apparatus was then immersed in the oil so that the oil level was at the bottom of the ground glass joints. The mixture was stirred until all of the solid monomer had melted and the internal temperature had reached approximately 125° C. At this time, a volume of solution of stannous octoate in chloroform equivalent to approximately 400 ppm (117 ppm Sn) was added to the melt using a syringe. The mixture was allowed to stir for approximately 3-16 hours. Then oil bath set point was decreased to approximately 125° C. and any residual unreacted monomer removed using vacuum over approximately 1 hour.

A 2500 ml portion of chloroform was used to dissolve and transfer the prepolymer to a pre-chilled, 20-liter jacketed reactor, which contained 2.5 equivalents (based on propylene glycol) of triethylamine and 0.5 equivalents of DMAP dissolved in 3600 ml of chloroform. The reactor was equipped with a stirrer bearing/shaft/turbine assembly, a gas joint, a tubing adapter, and a Teflon-coated thermocouple. With stirring and chilled recirculation on the jacket, the solution was cooled to below −15° C. A solution of 1 equivalent (based on propylene glycol, approximately 215 g) of distilled ethyl dichlorophosphate (EOPCl₂) in 650 ml chloroform was prepared in a 1000 ml 3-necked round bottom flask equipped with a tubing adapter and a gas joint. The EOPCl₂/chloroform solution was added using a piston pump and Teflon tubing over a period of 50 minutes, maintaining the internal temperature at approximately −10° C. Tubing was connected to the gas joints of the flask and reactor to equalize the pressure during the addition. Following the addition, a 50 ml portion of chloroform was added to rinse the flask, feed lines, and pump. The reaction mixture was stirred for 1 hour at low temperature (−8° C. after 1 hour) before the reaction was quenched with 140 ml of anhydrous methanol.

The reactor was then charged with 3 kg of Dowex DR-2030 IER and 3 kg of Dowex M-43 wetted with approximately 6.5 liters of methylene chloride. The polymer/resin mixture was mixed at low temperature for 3-15 hours, after which it was transferred by vacuum to a stainless steel laboratory Nutsche filter. After filtering off the resin, the polymer solution was pulled through the in-line 8 micron cartridge filter into the concentrator (a similar 10-liter jacketed reactor) where the solution was concentrated with the aid of heated recirculating fluid on the jacket. The 20-liter reactor and the resin in Nutsche were washed with 5 liters of methylene chloride, which were transferred to the concentrator after being stirred for 1 hour. An additional 5 liters of methylene chloride were added to the resin in the Nutsche and added to the concentrator when the solution had been reduced to approximately 6 liters.

Concentration of the polymer solution continued until approximately 4-5 liters of a viscous solution remained. A portion of 1500 ml of ethyl acetate was then added to the polymer solution. The mixture was mixed until homogenous and precipitated in approximately 10 liters of petroleum ether. After the precipitation mixture was stirred for approximately 5 minutes, the supernatant liquid was decanted. The polymer was then washed with 5 liters of petroleum ether. After the mixture was stirred for 5 minutes. The liquid was again decanted. The polymer was poured into a Teflon-coated pan and placed in the vacuum oven at NMT 50° C. After drying for 24 hours, the polymer was ground into smaller pieces and dried for additional time in a vacuum oven at ambient temperature.

### Example 12

### IUdR Size Reduction by Spray Drying

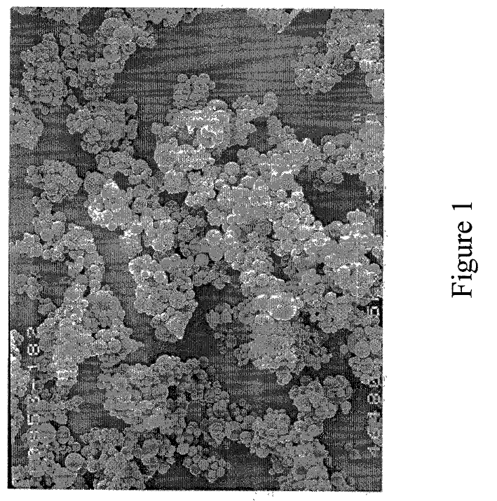

3.0 gram of IUdR was accurately weighed into a 2000 ml beaker, methanol was added to dissolve IUdR as 0.2% concentration (w/w). The clear solution was spray dried by using a mini spray dryer, Büchi B-191. The process conditions were: inlet temperature 63° C., outlet temperature 47° C., aspiration setting 100%, pump rate 4.6 gram/min, spray flow 600 L/hr. Drug particles were collected, and stored at 4° C. Spherical microspheres were achieved with typical yield of 20% to 30%. Volume weighted particle size median is 70 um, individual microspheres look to be about 2 to 3 um, the particle size results indicate aggregations of the micropsheres. The morphology of size-reduced IUdR particles was analyzed by Scanning Electron Microscopy (SEM), which is presented in FIG. 1.

### Example 13

### IUdR Size Reduction by Precipitation

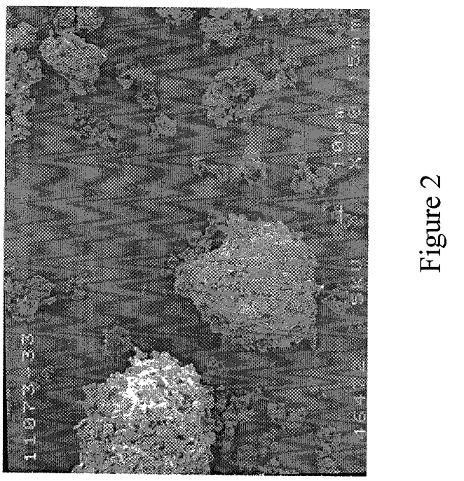

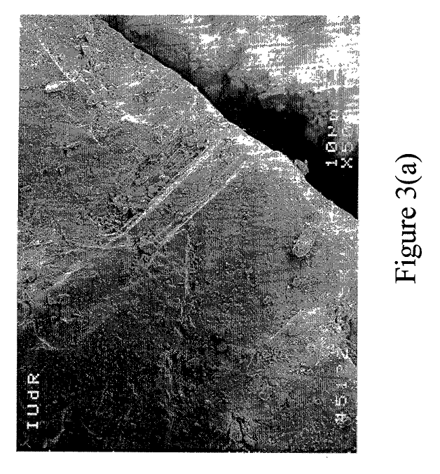

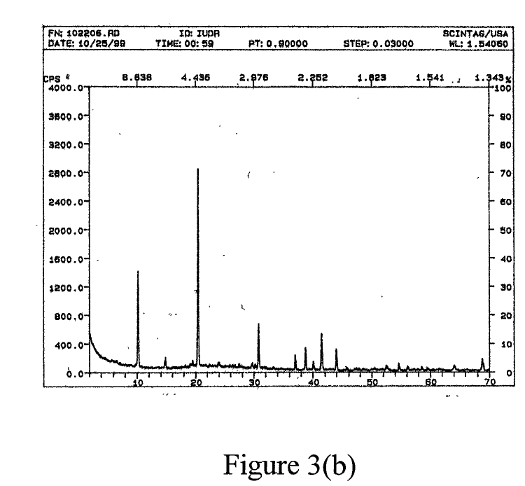

5.0 gram of IUdR was accurately weighed into a 20 ml scintillation vial, 15 ml of dimethyl sulfoxide was added to dissolve IUdR with vortexing. The light-yellow, clear solution was transferred into a 200 ml beaker before dichloromethane was added slowly. With sonication, the solution became cloudy once the volume ratio between dichloromethane and DMSO reached 7:1, and IUdR began to precipitate into the dichloromethane. Continue sonication for another 5 minutes after IUdR stopped precipitation. The mixture was allowed to settle at room temperature for 2 hours before the supernatant was decanted. The precipitation was freeze and dried for two days. The dried IUdR powder was grinded, and sieved through a 100 um sieve, collected, and stored at 4° C. The typical yield for this procedure was 60 to 70%. The volume weighted particle size median was 20 to 30 um. The morphology of size-reduced IUdR particles was analyzed by SEM, which is presented in FIG. 2. In order to compare the morphology change after size-reduction process, the SEM and X-Ray data for IUdR raw material is presented in FIGS. 3a and 3b respectively.

### Example 14

### Preparation of 20% IUdR/D,L-PL(PG)EOP Microspheres by Spray Drying Emulsion; In Vitro Release

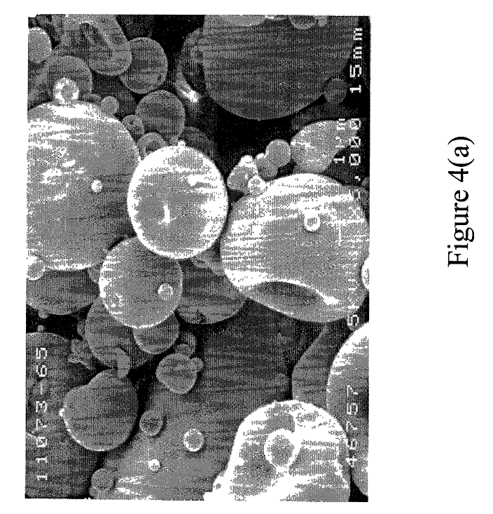

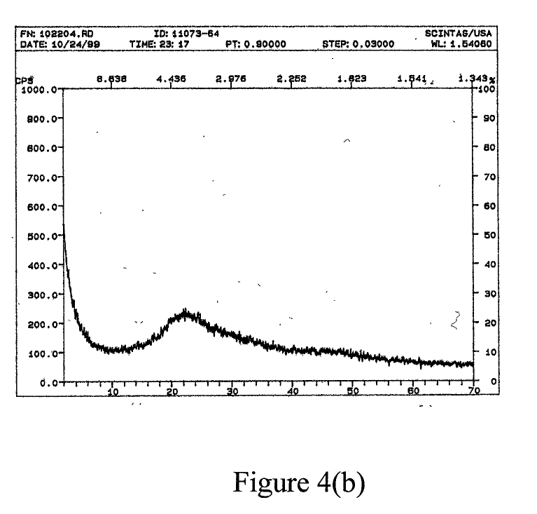

5.0 gram of D,L-PL(PG)EOP as prepared in Example 11 was accurately weighed into a 200 ml beaker, and dichloromethane was added to make 15% polymer concentration (w/w). 5.0 gram of IUdR was accurately weighed into a 20 ml scintillation vial, concentrated sodium hydroxide solution and 18 ml of deionized water were added to dissolve the IUdR (pH=13). Polymer and IUdR solutions were mixed, homogenized at speed setting of 40 on the vertic cyclone vertishear homogenizer for 2 to 3 minutes. With constant stirring, the milky emulsion was spray dried immediately with a mini spray dryer, Büchi B-191. The process conditions were: inlet temperature 45° C., outlet temperature 35° C., aspiration setting 70% to 80%, pump rate 8.0 gram/min, spray flow 600 L/hr. The spray dried products were collected and stored at 4° C. Roughly spherical microparticles were achieved with volume weighted particle size median around 25 um. SEM and X-Ray are presented in FIGS. 4(a) and 4(b).

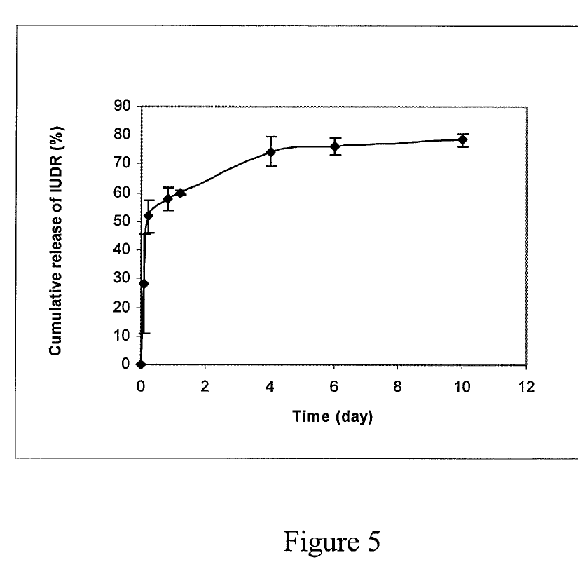

In triplicate, 20 mg of the spray dried materials was accurately weighed into 50 ml centrifuge tubes, 20 ml of PBS buffer (pH7.4, 0.1M) was added. At each specific time point, the buffer was replaced, and IUdR concentration was analyzed by a reversed phase HP-LC method at 282 um. In vitro release results are summarized in FIG. 5.

### Example 15

### Preparation of 50% IUdR/D,L-PL(PG)EOP Microspheres by Spray Drying Emulsion; In Vitro Release

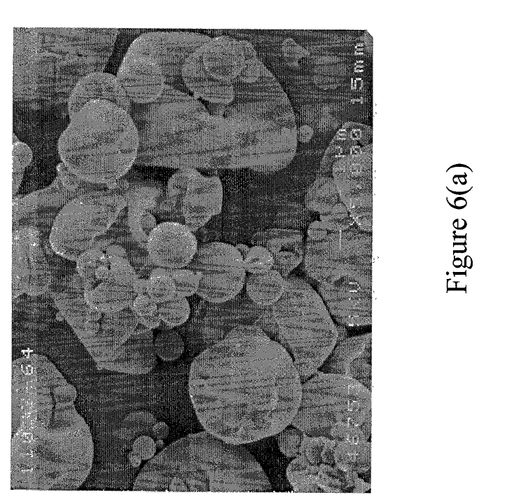

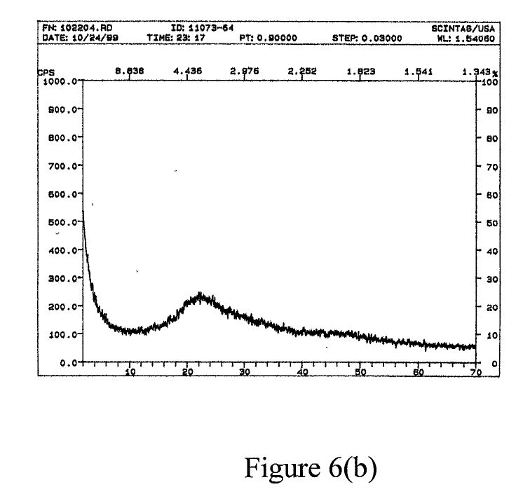

5.0 gram of D,L-PL(PG)EOP as prepared in Example 11 was accurately weighed into a 200 ml beaker, and dichloromethane was added to make 15% polymer concentration (w/w). 5.0 gram of IUdR was accurately weighed into a 20 ml scintillation vial, concentrated sodium hydroxide solution and 18 ml of deionized water were added to dissolve the IUdR (pH=13). Polymer and IUdR solutions were mixed, homogenized at speed setting of 40 on the vertic cyclone vertishear homogenizer for 2 to 3 minutes. With constant stirring, the milky emulsion was spray dried immediately with a mini spray dryer, Büchi B-191. The process conditions were: inlet temperature 45° C., outlet temperature 35° C., aspiration setting 70% to 80%, pump rate 8.0 gram/min, spray flow 600 L/hr. The spray dried products were collected and stored at 4° C. Roughly spherical microparticles were achieved with volume weighted particle size median around 25 um. SEM and X-Ray are presented in FIGS. 6(a) and 6(b).

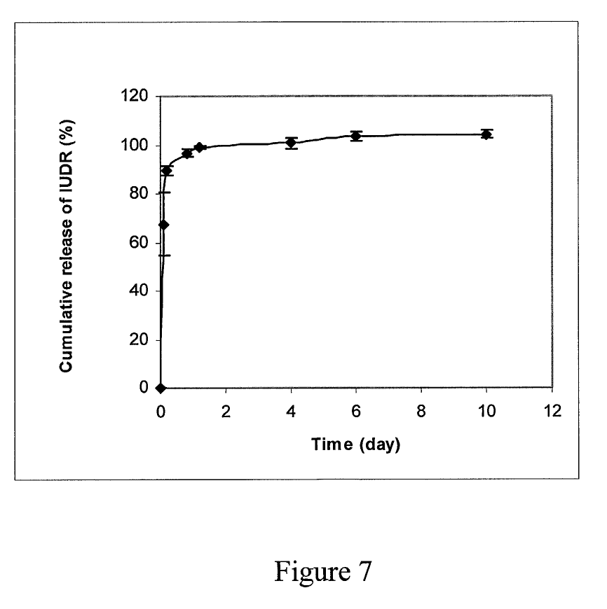

In triplicate, 20 mg of the spray dried materials was accurately weighed into 50 ml centrifuge tubes, 20 ml of PBS buffer (pH7.4, 0.1M) was added. At each specific time point, the buffer was replaced, and IUDR concentration was analyzed by a reversed phase HPLC method at 282 nm. In vitro release result is summarized in FIG. 7.

### Example 16

### Preparation of 15% IUdR/D,L-PL(PG)EOP Microspheres by Spray Drying Dispersion

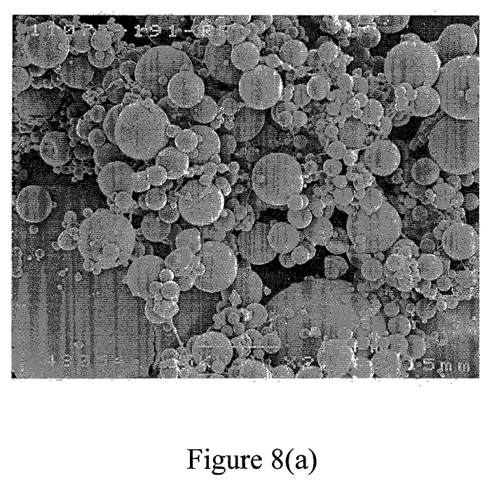

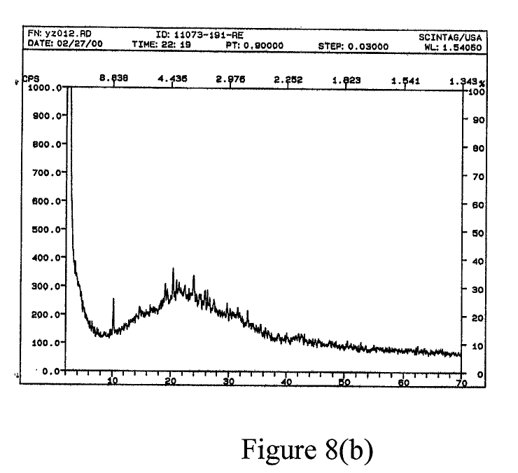

6.0 gram of D,L-PL(PG)EOP as prepared in Example 11 was accurately weighed into a 200 ml beaker, dichloromethane was added to make a 20% polymer solution (w/w). 1.05 gram of size-reduced IUdR (obtained from Example 2) was accurately weighed into a 20 ml scintillation vial. The polymer solution and IUdR powder were mixed, followed by homogenization for 3 minutes at speed setting of 40 on vertic cyclone vertishear homogenizer. With constant stirring, the dispersion mixture was spray dried immediately with a mini spray dryer, Büchi B-191. The process conditions were: aspiration setting 70% to 80%, pump rate 8.0 gram/min, spray flow 600 L/hr. The spray dried products were collected and stored at 4° C. Spherical microparticles were achieved with volume weighted particle size median around 10 um. SEM and X-Ray are presented FIGS. 8(a) and 8(b) respectively, indicating spherical microspheres with some crystals present.

### Example 17

### Preparation of 23% IUdR/D,L-PL(PG)EOP Microspheres with Dilution Method; In Vitro Release

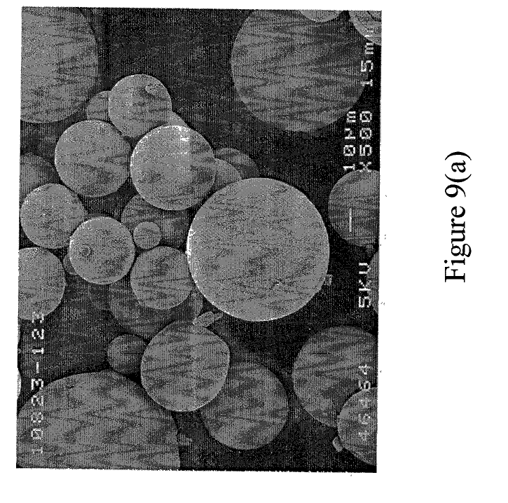

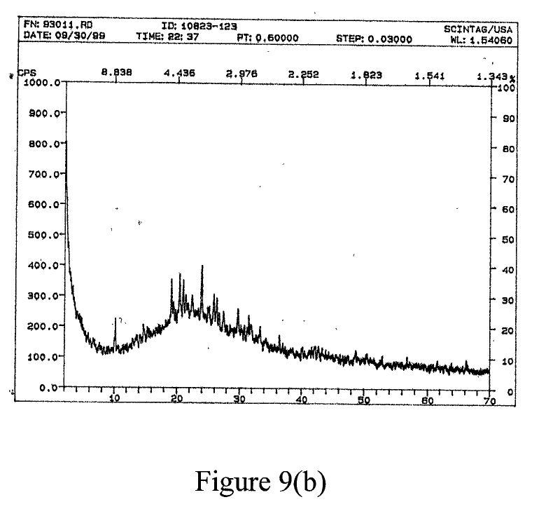

1.6 gram of IUdR was accurately weigh into a 20 ml scintillation vial, 5 ml of DMSO and 2 ml of acetone were added to dissolve the drug. 3.3 gram of D,L-PL(PG)EOP as prepared in Example 11 was accurately weighed and dissolved into dichloromethane as 50% concentration (w/w). The drug and polymer solutions were mixed and added to 200 ml of 0.5% PVA solution with agitation using Virtishear homogenizer at 60 setting for about 1 minutes. The mixture was immediately diluted with 200 ml of deionized water and stirred for 5 minutes and subsequently diluted to 1000 ml total volume with deionized water and stirred for another 20 minutes. The microspheres were collected, rinsed and lyophilized for 4 days. Spherical microparticles were achieved with volume weighted particle size median of 82 um. Encapsulation rate of IUdR was determined as 71%. The SEM and X-Ray results are presented in FIGS. 9(a) and 9(b) respectively, indicating dense and spherical microspheres with some crystals present.

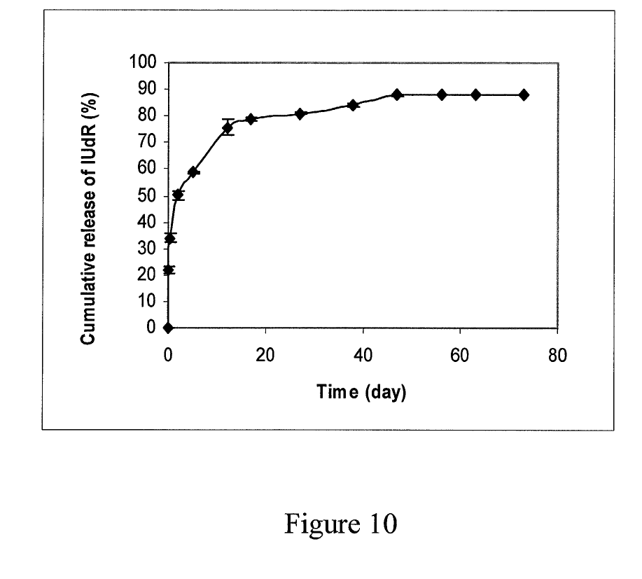

In triplicate, accurately weighed 20 mg of the microspheres into 50 ml centrifuge tubes, 20 ml of PBS buffer (pH7.4, 0.1M) was added. At each specific time point, the buffer was replaced, and IUdR concentration was analyzed by a reversed phase HPLC method at 282 nm. The in vitro release result is summarized in FIG. 10.

### Example 18

### Preparation of 20% IUdR/D,L-PL(PG)EOP Rod; In Vitro Release

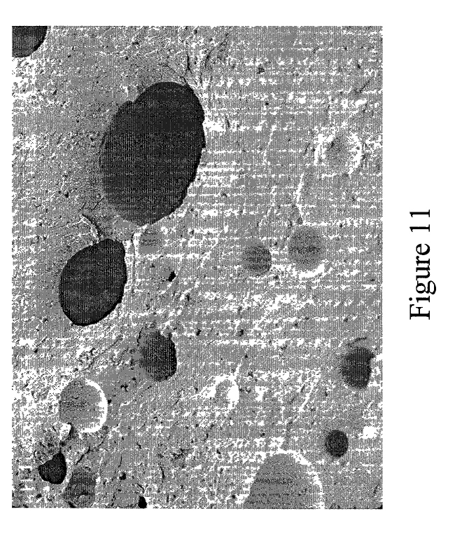

Preheat the oven at 80° C. to 90° C. Spray dried 50% IUdR/D,L-PL(PG)EOP microspheres (obtained from Example 14) were loaded into a 1 ml Hamilton syringe with the plunger removed. The sample syringe was placed into the oven for 5 minutes or until the microspheres were melted. The melt was compressed using the plunger until resistance was felt. Cool down the syringe at room temperature before cut into uniform pieces for characterization and in vitro release of IUdR. The typical size of the rod was 4.70 mm in diameter, and 1.5 mm for thickness. The morphology result is presented in FIG. 11.

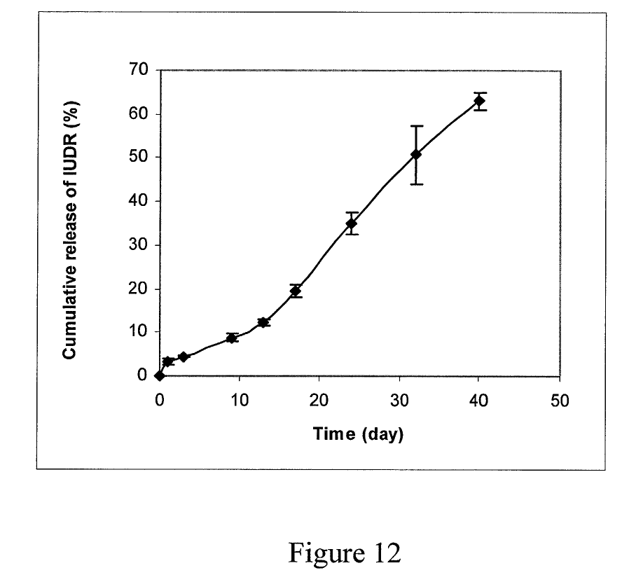

In triplicate, accurately weighed 20 mg of the rod into 50 ml centrifuge tubes, 20 ml of PBS buffer (pH7.4, 0.1M) was added. At each specific time point, the buffer was replaced, and IUdR concentration was analyzed by a reversed phase HPLC method at 282 um. The in vitro release result is summarized in FIG. 12.

### Example 19

### Preparation of 50% IUdR/D,L-PL(PG)EOP Rod: In Vitro Release

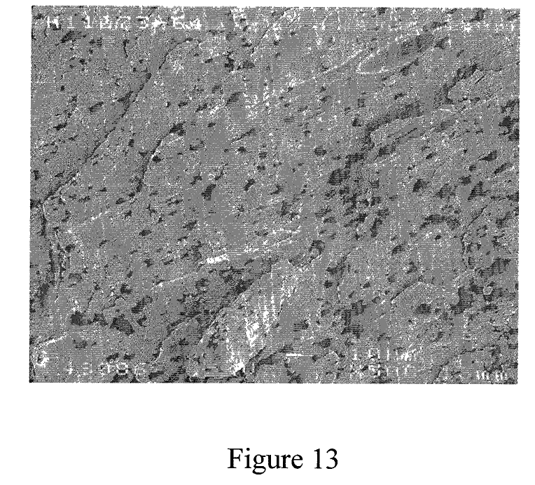

Preheat the oven at 80° C. to 90° C. Spray dried 50% IUdR/D,L-PL(PG)EOP microspheres (obtained from Example 15) were loaded into a 1 ml Hamilton syringe with the plunger removed. The sample syringe was placed into the oven for 5 minutes or until the microspheres were melted. The melt was compressed using the plunger until resistance was felt. Cool down the syringe at room temperature before cut into uniform pieces for characterization and in vitro release of IUdR. The typical size of the rod was 4.70 mm in diameter, and 1.5 mm for thickness. The morphology result is presented in FIG. 13.

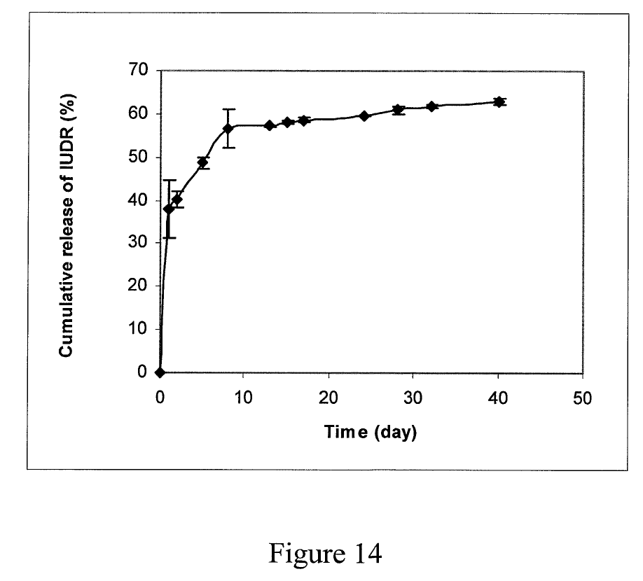

In vitro release of IUdR. In triplicate, accurately weighed 20 mg of the rod into 50 ml centrifuge tubes, 20 ml of PBS buffer (pH7.4, 0.1M) was added. At each specific time point, the buffer was replaced, and IUdR concentration was analyzed by a reversed phase HPLC method at 282 um. The in vitro release result is summarized in FIG. 14.

### Example 20

### Preparation of 20% IUdR/D,L-PL(PG)EOP Microparticles; In Vitro Release

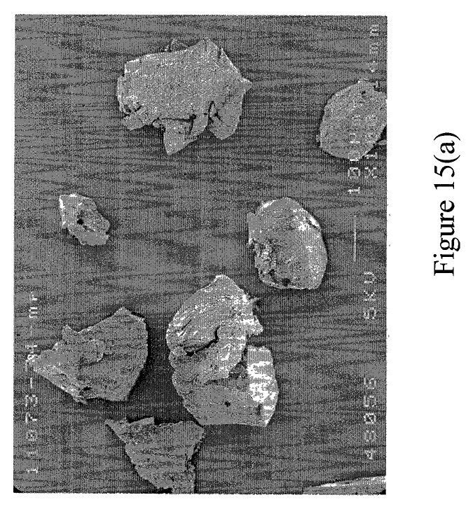

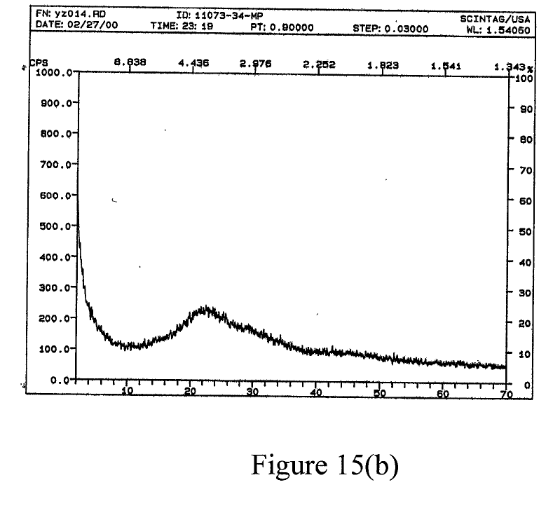

Preheat the oven at 80° C. to 90° C. Spray dried 20% IUdR/D,L-PL(PG)EOP microspheres (obtained from Example 14) were loaded into a 1 ml Hamilton syringe with the plunger removed. The sample syringe was placed into the oven for 5 minutes or until the microspheres were melted. The melt was compressed out of the syringe, grinded, sieved through a 250 um sieve, collected and stored at 4° C. The microparticles have volume weighted particle size median of 91 um. The SEM and X-Ray data are presented in FIGS. 15(a) and 15(b) respectively, indicating no crystals present.

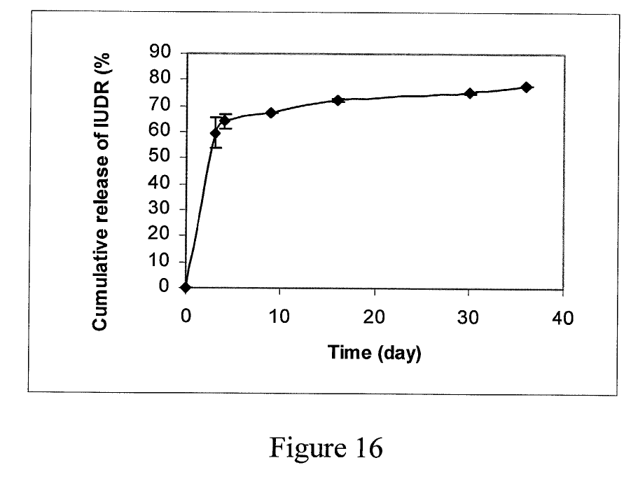

In triplicate, 20 mg of the microparticles was accurately weigh into 50 ml centrifuge tubes, 20 ml of PBS buffer (ph7.4, 0.1M) was added. At each specific time point, the buffer was replaced, and IUdR concentration was analyzed by a reversed phase HPLC method at 282 nm. The in vitro release result is presented in FIG. 16.

### Example 21

### Preparation of 50% IUdR/D,L-PL(PG)EOP Microparticles; In Vitro Release

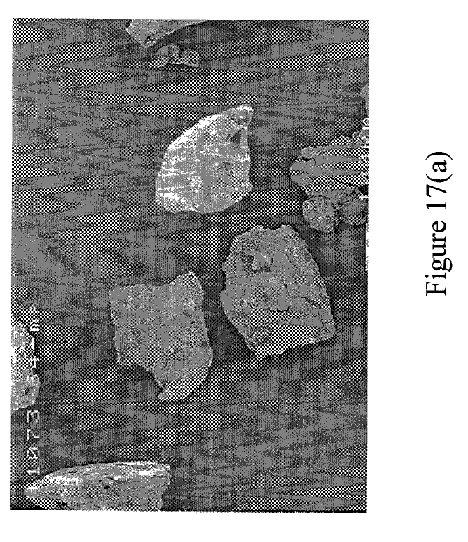

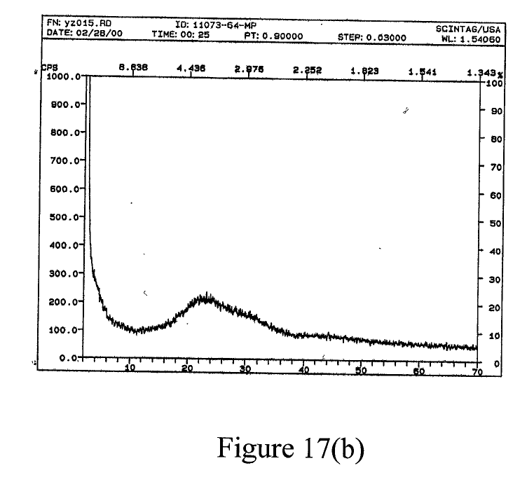

Preheat the oven at 80° C. to 90° C. Spray dried 50% IUdR/D,L-PL(PG)EOP microspheres (obtained from Example 15) were loaded into a 1 ml Hamilton syringe with the lunger removed. The sample syringe was placed into the oven for 5 minutes or until the microspheres were melted. The melt was compressed out of the syringe, grinded, sieved through a 250 um sieve, collected and stored at 4° C. The microparticles have volume weighted particle size median of 133 um. The SEM and X-Ray results are presented in FIGS. 17(a) and 17(b) respectively, indicating no crystals present.

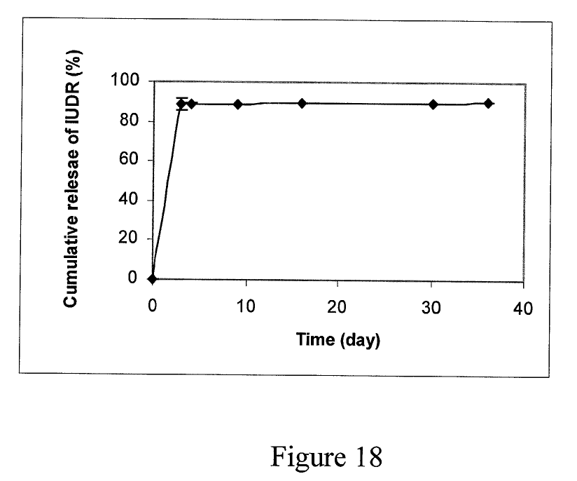

In triplicate, 20 mg of the microparticles was accurately weigh into 50 ml centrifuge tubes, 20 ml of PBS buffer (ph7.4, 0.1M) was added. At each specific time point, the buffer was replaced, and IUdR concentration was analyzed by a reversed phase HPLC method at 282 nm. The in vitro release result is presented in FIG. 18.

### Example 22

### Preparation of 25% IUdR/P(trans-CHDM/HOP) Paste; In Vitro Release

250 mg of size-reduced IUdR (obtained from Example 12) was accurately weighed into a 20 ml scintillation vial, 750 mg of P(trans-CHDM/HOP) using the same molar ratios as in Example 8 was accurately weighed into another 20 ml scintillation vial. IUdR powder and polymer were mixed using physical blending until visible uniformity was achieved.

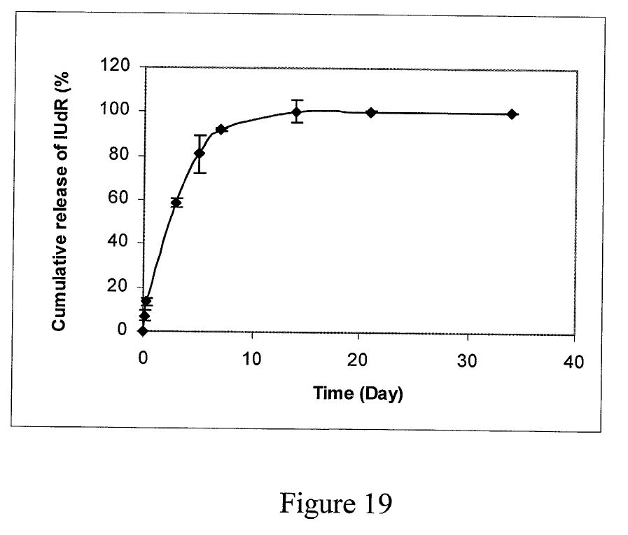

In triplicate, 20 mg of the paste was accurately weighed into 50 ml centrifuge tubes, 20 ml of PBS buffer (pH7.4, 0.1M) was added. At each specific time point, the buffer was replaced, and IUdR concentration was analyzed by a reversed phase HPLC method at 282 nm. The in vitro release result is summarized in FIG. 19. IUdR was completely released within ten days following a typical first-order kinetics.

### Example 23

### Preparation of 20% IUdR/P(BHET-EOP/TC) Film with Dispersion; In Vitro Release

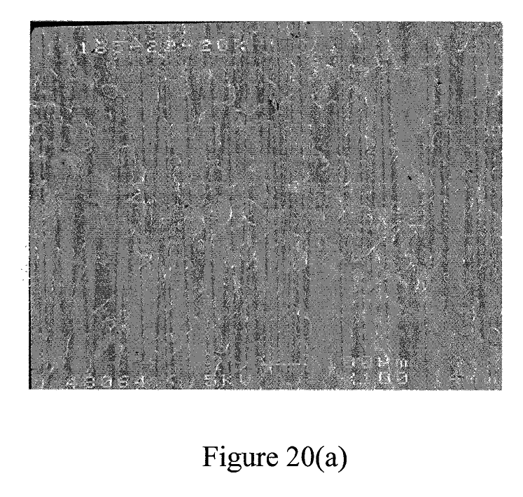

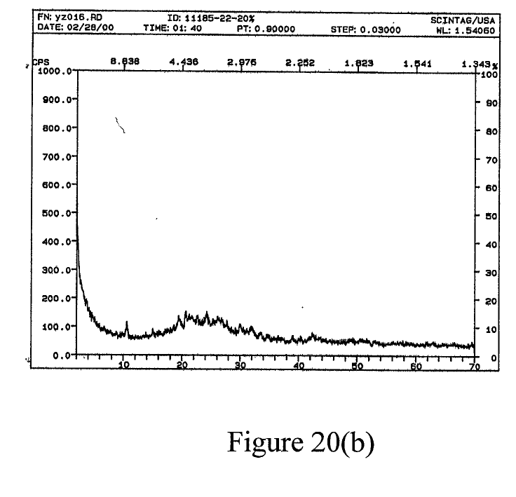

2.0 gram of P(BHET-EOP/TC) using the same molar ratios as for Example 10 was accurately weighed into a 20 ml scintillation vial, dichloromethane was added to dissolve the polymer as 50% polymer concentration (w/w). 500 mg of IUdR was accurately weighed into another 20 ml of scintillation vial. Polymer solution and IUdR powder were mixed, and vortexed until a stable dispersion was achieved. The mixture was poured onto a chilled Teflon mold. The Teflon mold was covered with aluminum foil, air dried at room temperature overnight, and lyophilized for two days. The SEM and X-Ray results are presented in FIGS. 20(a) and 20(b) respectively, indicating roughness of the surface, and some crystals present.

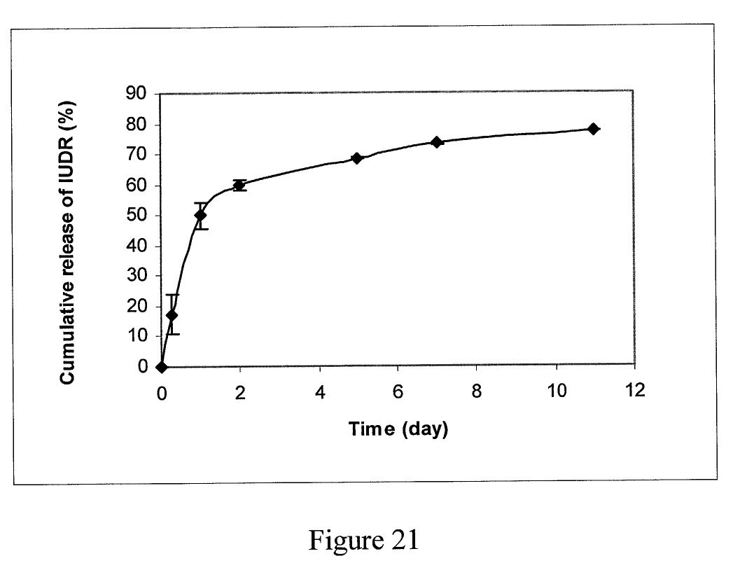

In triplicate, the dried films were cut into small wafers with 3/32 inch of punch and die, accurately weighed into 50 ml centrifuge tubes, 20 ml of PBS buffer (pH7.4, 0.1M) was added. At each specific time point, the buffer was replaced, and IUdR concentration was analyzed by a reversed phase HPLC method at 282 nm. The in vitro release result is summarized in FIG. 21.

### Example 24

### Preparation of 50% IUdR/P(BHET-EOP/TC) Film with Dispersion; In Vitro Release

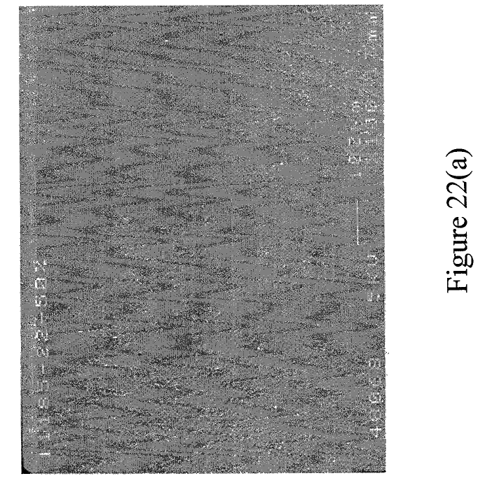

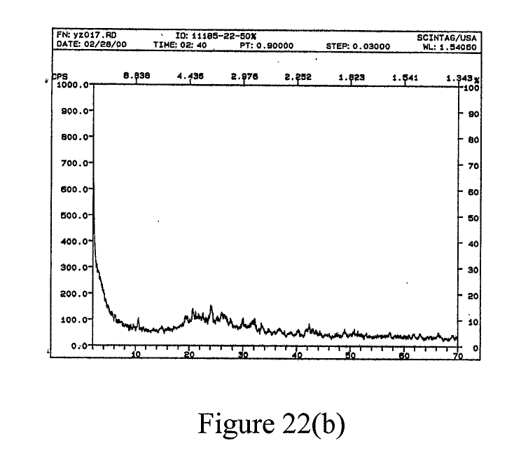

2.0 gram of P(BHET-EOP/TC) using the same molar ratios as for Example 10 was accurately weighed into a 20 ml scintillation vial, dichloromethane was added to dissolve the polymer as 50% polymer concentration (w/w). 2.0 gram of size-reduced IUdR (obtained from Example 2) was accurately weighed into another 20 ml of scintillation vial. Polymer solution and IUdR powder were mixed, and vortexed until a stable dispersion was achieved. The mixture was poured onto a chilled Teflon mold. The Teflon mold was covered with aluminum foil, air dried at room temperature overnight, and lyophilized for two days. The SEM and X-Ray are presented in FIGS. 22(a) and 22(b) respectively, indicating some crystals present.

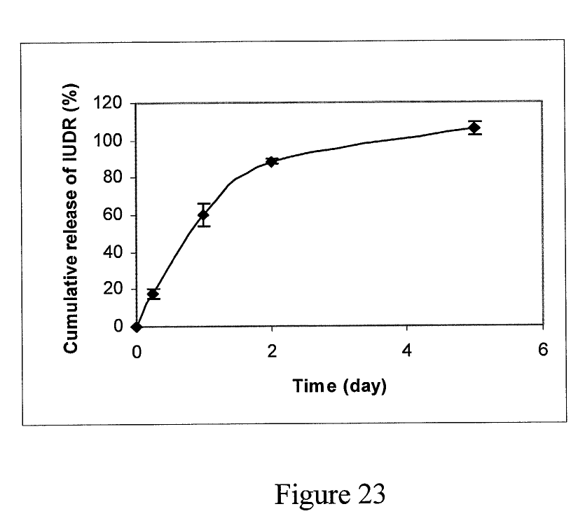

In triplicate, the dried films were cut into small wafers with 3/32 inch of punch and die, accurately weighed into 50 ml centrifuge tubes, 20 ml of PBS buffer (pH7.4, 0.1M) was added. At each specific time point, the buffer was replaced, and IUdR concentration was analyzed by a reversed phase HPLC method at 282 nm. The in vitro release result is summarized in FIG. 23.

### Example 25

### Preparation of 20% IUdR/P(BHET-EOP/TC) Film with Co-Solvent

2.0 gram of P(BHET-EOP/TC) using the same molar ratios as for Example 10 was accurately weighed into a 20 ml scintillation vial, dichloromethane was added to dissolve the polymer as 50% polymer concentration (w/w). 500 mg of IUdR was weighed into another 20 ml scintillation vial, dimethyl sulfoxide was gradually added to dissolve IUdR as a clear, light yellow solution. Polymer and IUdR solutions were mixed well and poured onto a chilled Teflon mold. The Teflon mold was covered with aluminum foil, air dried at room temperature overnight, and lyophilized for 2 days.

### Example 26

### Preparation of 50% IUdR/P(BHET-EOP/TC) Film with Co-Solvent

1.25 gram of P(BHET-EOP/TC) using the same molar ratios as for Example 10 was accurately weighed into a 20 ml scintillation vial, dichloromethane was added to dissolve the polymer as 50% polymer concentration. 1.25 gram of IUdR was accurately weighed into another 20 ml of scintillation vial, dimethyl sulfoxide was gradually added to dissolve IUdR as a clear, light yellow solution. Polymer and IUdR solutions were mixed well and poured onto a chilled Teflon mold. The Teflon mold was covered with aluminum foil, air dried at room temperature overnight, and lyophilized for 2 days.

### Example 27

### In Vitro Radiosensitivity

To have log phase growth, U251 human malignant glioma cells were trypsinized and re-plated in triplicate three days prior to irradiation in media containing either no radiosensitizer (control) or 10 μM IUdR (Sigma, St. Louis, Mo.). The cells were acutely (1.1 gy/min) irradiated (AECL Gamma-cell 40 irradiator, Canada) with increasing single fractions (0, 2.5, 5.0 or 10 gy).

Immediately after irradiation, the cells were trypsinized, counted, and re-plated in media having no IUdR in numbers to yield between 20 and 200 colonies per plate. After 10 days, the plates were fixed with methanol and acetic acid, stained with crystal violet, and scored for colonies containing more than 50 cells. The resulting radiation survival data from IUdR-treated cells were corrected for plating efficiency in IUdR alone.

### Example 28

### In Vivo Animal Studies using IUdR Loaded D,L-PL(PG)EOP

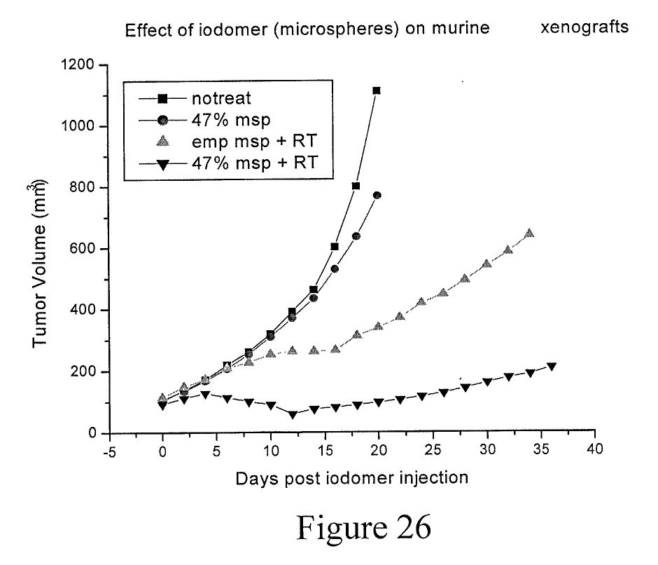

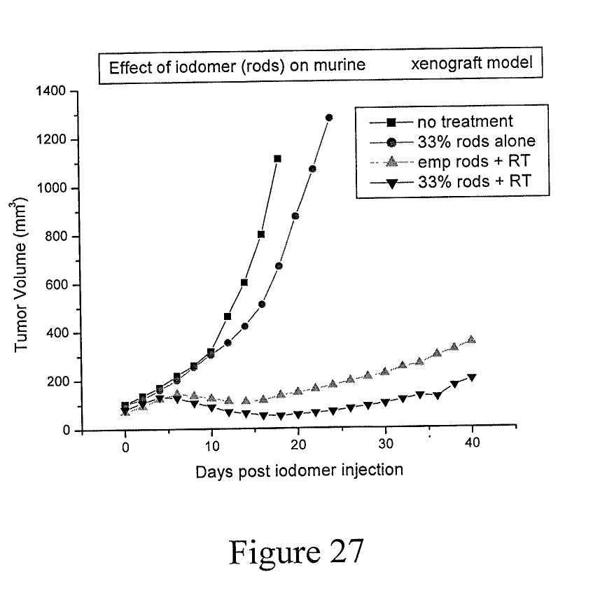

The efficacy of the subject compositions was tested in the presence and absence of radiation in a murine cancer orthotopic model. 1×10⁶ SCC VII/SF cells were injected subcutaneously on the right flank of C3H mice. Seven days post cell injection, tumors were approximately 100 mm³. Microspheres and rods of D,L-PL(PG)EOP prepared as described above in Example 11 and otherwise, with 47% loading and 33 loading of IUdR, respectively, were injected directly into the tumor. As appropriate, 24 hours after injection, tumors were irradiated 4 gy/day for 5 consecutive days. Each of the microspheres and rods at the different loading levels were compared to no treatment control, loaded rods/microspheres minus RT, placebo rods/microspheres plus RT. As shown in FIGS. 26 and 27, loaded rods/microspheres plus RT was significantly better (p<0.001) at decreasing tumor size compared to all controls.

In another in vivo experiment, 1×10⁷ cells from the human cancer cell line JHU 012 were injected into the anterior floor of the mouth of immunocompromised (nude) mice. Ten days following injection, animals were anesthetized, tumors measured, intumoral injections of the same compositions identified above were completed followed by the same radiation treatment (as appropriate). At approximately twenty days after injection, animals sacrificed and tumors measured. The following approximate tumor sizes (mm³) were observed: no treatment, 750; radiation alone, 300; empty microspheres, 500; 47% IUdR microspheres (1.175 mg IUdR in injection volume of 50 ul), 425; empty rods, 250; 33% IUdR rods, 350; and direct intrumoral injection of IUdR (0.065 mg to 0.12 mg in injection volume of 50 ul), 100.

### Example 29

### Treatment Using Subject Polymer Compositions

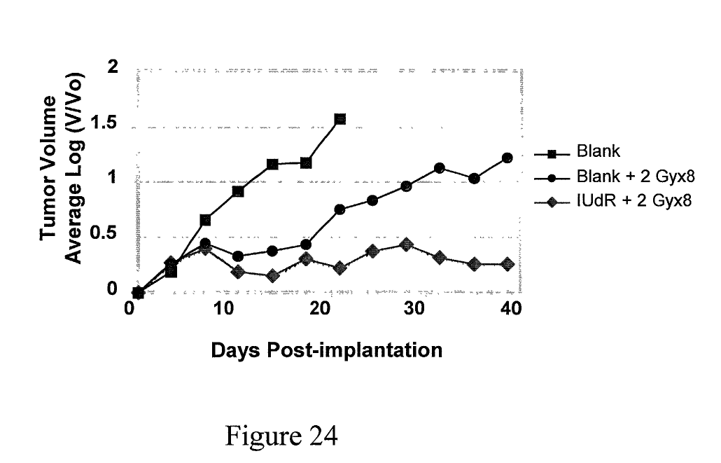

The effect of irradiation in conjunction with radiosensitizers delivered by P(trans-CHDM/HOP) paste was tested in a U251 xenograft model. The polymer had a weight average molecular weight of 8080, and number average molecular weight of 3100 (polystyrene as calibration standards). P(trans-CHDM-HOP) had a viscosity of 209 cps at room temperature. The drug IUdR was blended into the polymer paste at room temperature and at a loading level of 16.7 weight %. As shown in FIG. 24, growth delay of the flank xenograft was observed in animals receiving such paste in addition to external beam irradiation. Animals were irradiated on day 4 after implantation, with 2 Gy twice a day for 4 days.

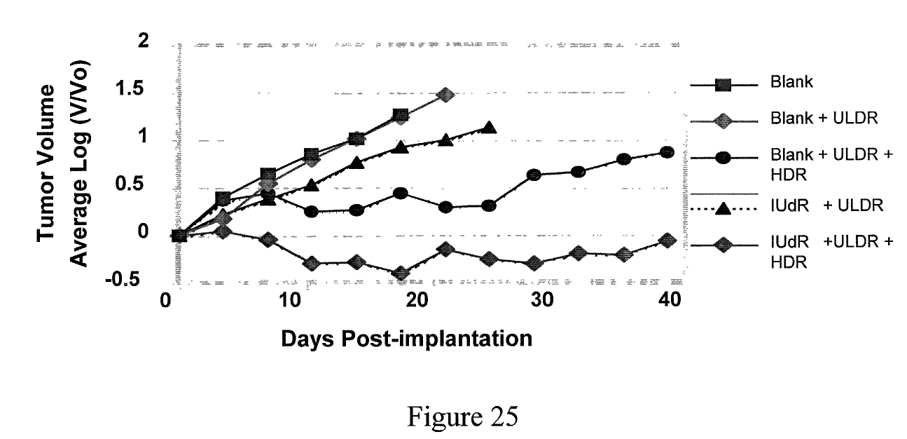

To test the effect of protracted exposure sensitization, a 137-Cs source was mounted a fixed distance above the cages of animals bearing xenografts for whole body continuous ULDR (as defined below). FIG. 25 shows that ULDR alone has minimal beneficial effect on growth delay. Combining that with HDR leads to a significant delay, which is more potent than the combination of IUdR and ULDR. Most remarkable results, with tumor regression, however, was observed with the combination of IUdR, ULDR, and HDR. These results confirm the hypothesis that the combined IUdR administration via polymeric controlled delivery, continuous ULDR and fractionated HDR treatments may markedly increase cell killing and growth delay in vivo. For purposes of this experiment, ULDR=0.03 Gy/hr for 72 hr started 24 hours after xenograft transplantation, and HDR=2 Gy twice a day for 4 days, started immediately after ULDR, which is 4 days after implantation.

References

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Patents and patent applications: U.S. Pat. Nos. 4,638,045, 4,757,128, 4,857,311, 4,866,168, 4,886,870, 4,906,474, 4,946,929, 5,194,581, 5,256,715, 5,620,883, 5,626,862, 5,176,907, 5,219,564, 5,099,060, 5,900,249, 5,747,060, 5,505,922, 5,856,342, 5,747,060, 5,633,000, 5,686,091, 5,759,582, 5,846,565, 5,849,738, 5,858,388, 5,861,159, 5,912,225, 5,954,439, 5,958,947, 5,972,707, 6,008,318, 5,942,241, 5,942,543, 5,922,340, 6,075,059, 6,031,007, 6,045,824, 6,046,187, and 5,993,836.

Publications and other references: Andersson-Engels et al, Anal. Chem. 62(1), 19A-27A (1990); Ertel et al., (1995) J. Biomedical Materials Res. 29:1337-1348; Choueka et al., (1996) J. Biomed. Materials Res., 31:35-41; Kessel, IEEE J. QUANTUM ELECTRON., QE 23(10):1718-20 (1987); Langer et al., (1983) Rev. Macro. Chem. Phys. C23(1):61; Leong et al., (1986) Biomaterials, 7:364; Leong, et al., J. Med. Biomed. Mater. Res. 19, 941 (1985); Leong, et al., J. Med. Biomed. Mater. Res. 20, 51 (1986); North et al., Blood Cells, 18:129-40 (1992); Penczek et al., Handbook of Polymer Synthesis Chapter 17: “Phosphorus-Containing Polymers”, (Hans R. Kricheldorf ed., 1992); Stein, C., (1988) Pharm. Biochem. Behavior, 3 1:445-451; Reza et al., J. Microencapsulation, 15(6):789-801 (1998); Rosen, et al., Biomaterials 4, 131 (1983); Suto et al., (1991) Anticancer Drug Des., 6:107-17; Williams et al., (1997) J. of Neuro-Oncology, 32:181-92; Williams et al., J. Radiat Oncol Biol Phys, 43(3):631-9 (1998); Van Gool et al., (1997) Eur. J. Biochem., 244:15-20.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A composition suitable for administration to a patient for treating a neoplasm, said composition comprising:
(a) a biocompatible polymer having phosphorous-based linkages; and
(b) one or more radiosensitizers in an aggregate amount equal to at least five percent by weight of said composition,
wherein a single dose of said composition provides extended release of at least one of said radiosensitizers over a period of at least about one day, and
wherein said composition is effective to inhibit the growth of said neoplasm upon (i) administration of said composition to said patient such that said composition is in at least partial contact with said neoplasm or tissue surrounding the site of said neoplasm, and (ii) subsequent treatment of said patient with electromagnetic radiation.

2. The composition of claim 1, wherein said amount is at least about 10% by weight of said composition.

3. The composition of claim 1, wherein said inhibition of said growth of said neoplasm is measured as a delay in doubling time as compared to no treatment.

4. The composition of claim 3, wherein said amount is at least about 10% by weight of said composition and wherein said doubling time is extended by a factor of at least about two.

5. The composition of claim 3, wherein said amount is at least about 30% by weight of said composition and wherein said doubling time is extended by a factor of at least about two.

6. The composition of claim 3, wherein said amount is at least about 20% by weight of said composition and wherein said doubling time is extended by a factor of at least about three.

7. The composition of claim 4, wherein said doubling time is extended by a factor of at least about four.

8. The composition of claim 1, wherein said inhibition of said growth of said neoplasm is measured by a reduction in the volume of said neoplasm as compared to no treatment on a date approximately thirty days after administration of said composition.

9. The composition of claim 8, wherein said amount is at least about 20% by weight of said composition and wherein said reduction in said volume is at least about 10%.

10. The composition of claim 8, wherein said reduction in said volume is at least about 10% on a date approximately sixty days after said administration.

11. The composition of claim 8, wherein said amount is at least about 30% by weight of said composition and wherein said reduction in said volume is at least about 30%.

12. The composition of claim 8, wherein said reduction in said volume is at least about 30% on a date approximately sixty days after said administration.

13. The composition of claim 8, wherein said reduction in said volume is at least about 50%.

14. The composition of claim 8, wherein said reduction in said volume is at least about 70%.

15. The composition of claim 2, wherein a single dose of said composition provides extended release of at least one of said radiosensitizers over a period of at least about 15 days.

16. The composition of claim 1, wherein a single dose of said composition provides extended release of at least one of said radiosensitizers over a period of at least about 30 days.

17. The composition of claim 1, wherein said composition releases a therapeutically effective amount of at least one of said radiosensitizers over a period of at least about three growth cycles of said neoplasm.

18. The composition of claim 1, wherein said amount is at least about 30% by weight of said composition and wherein said composition releases a therapeutically effective amount of at least one of said radiosensitizers over a period of at least about three growth cycles of said neoplasm.

19. The composition of claim 1, wherein said amount is at least about 30% by weight of said composition.

20. The composition of claim 1, wherein the therapeutic index for a course of electromagnetic radiation for treating said neoplasm is at least about three times greater when said composition is administered to said patient before said course.

21. The composition of claim 2, wherein the ED₅₀ for a course of electromagnetic radiation for treating said neoplasm is at least about three times greater when said composition is administered to said patient before said course.

22. The composition of claim 1, wherein said composition is in at least partial contact with said neoplasm or tissue surrounding the site of said neoplasm.

23. The composition of claim 1, wherein said composition is formulated as microspheres.

24. The composition of claim 23, wherein the mean diameter of said microspheres is less than about 250 microns.

25. The composition of claim 23, wherein the mean diameter of said microspheres is less than about 100 microns.

26. The composition of claim 2, wherein said composition is formulated as a solid particle or rod.

27. The composition of claim 23, wherein said microspheres are mixed with a pharmaceutically acceptable carrier.

28. The composition of claim 1, wherein said polymer is biodegradable.

29. The composition of claim 1, wherein said polymer has five or more units represented by the following formula: 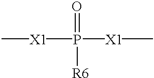
wherein, independently for each occurrence of said monomeric unit:
X1, each independently, represents —O— or —N(R5)-;
R5 represents —H, aryl, alkenyl or alkyl; and
R6 is any non-interfering substituent.

30. The composition of claim 29, wherein each occurrence of X1 for each of said units represents O.

31. The composition of claim 29, wherein each occurrence of R6 for each of said units represents H, alkyl, —O-alkyl, —O-cycloalkyl, aryl, —O-aryl, heterocycle or —O-heterocycle.

32. The composition of claim 2, wherein said polymer has two or more monomeric units represented by the following Formula V: 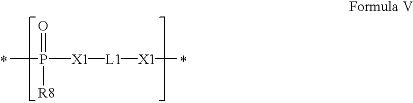
wherein, independently for each occurrence of said monomeric unit:
X1, each independently, represents —O— or —N(R7)-;
R7 represents —H, aryl, alkenyl or alkyl;
L1 represents any chemical moiety that does not materially interfere with the biocompatibility of said polymer;
R8 represents —H, alkyl, —O-alkyl, —O-cycloalkyl, aryl, —O-aryl, heterocycle, —O-heterocycle, or —N(R9)R10;
R9 and R10, each independently, represent a hydrogen, an alkyl, an alkenyl, —(CH₂)m—R11, or R9 and R10, taken together with the N atom to which they are attached complete a heterocycle having from 4 to about 8 atoms in the ring structure;
m represents an integer in the range of 0-10; and
R11 represents —H, alkyl, aryl, cycloalkyl, cycloalkenyl, heterocycle or polycycle.

33. The composition of claim 32, wherein said polymer comprises at least about five of said monomeric units.

34. The composition of claim 33, wherein all X1 are O.

35. The composition of claim 34, wherein L1 for at least a plurality of said units has 2 to about 20 atoms of carbon, oxygen, sulfur and nitrogen, wherein at least 60 percent of said atoms are carbon.

36. The composition of claim 1, wherein said polymer has one or more monomeric units represented by the following Formula VI: 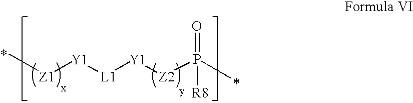
wherein Z1 and Z2, respectively, for each independent occurrence is: 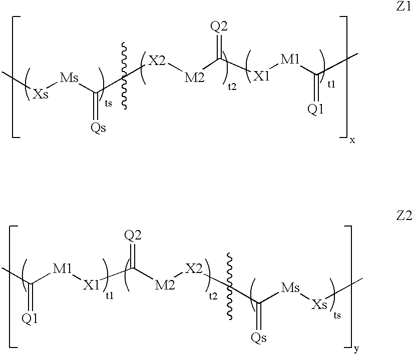
wherein, independently for each occurrence of said monomeric unit:
Q1, Q2 . . . Qs, each independently, represent —O— or —N(R7);
X1, X2 . . . Xs, each independently, represent —O— or —N(R7);
R7 represents —H, aryl, alkenyl or alkyl;
the sum of t1, t2 . . . ts is an integer and equal to at least one or more;
Y1 represents —O—, —S— or —N(R7)—;
x and y are each independently integers from 1 to about 1000 or more;
L1 represents any chemical moiety that does not materially interfere with the biocompatibility of said polymer;
M1, M2 . . . Ms each independently, represents any chemical moiety that does not materially interfere with the biocompatibility of said polymer;
R8 represents —H, alkyl, —O-alkyl, —O-cycloalkyl, aryl, —O-aryl, heterocycle, —O-heterocycle, or —N(R9)R10;
R9 and R10, each independently, represent a hydrogen, an alkyl, an alkenyl, —(CH₂)m—R11, or R9 and R10, taken together with the N atom to which they are attached complete a heterocycle having from 4 to about 8 atoms in the ring structure;
m represents an integer in the range of 0-10; and
R11 represents —H, alkyl, aryl, cycloalkyl, cycloalkenyl, heterocycle or polycycle.

37. The composition of claim 36, wherein said monomeric units comprise at least about 95 percent of the repeating units of said polymer.

38. The composition of claim 37, wherein the average molar ratio of (x or y):L1, when ts is equal to one, is from about 10:1 to about 4:1.

39. The composition of claim 37, wherein L1 represents a divalent branched or straight chain or cyclic aliphatic group or divalent aryl group.

40. The composition of claim 37, wherein each Q1, Q2 . . . Qs and each X1, X2 . . . Xs of each of said monomeric units of said polymer is O and the sum of t1, t2 . . . ts equals one for each of Z1 and Z2.

41. The composition of claim 36, wherein each M1, M2 . . . Ms of each of said monomeric units of said polymer represents a divalent aliphatic moiety having from 1 to about 7 carbon atoms.

42. The composition of claim 36, wherein said monomeric units are represented by the following Formula VIf:

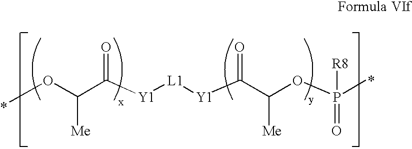

43. The composition of claim 36, wherein each of Z1 and Z2 is represented by: 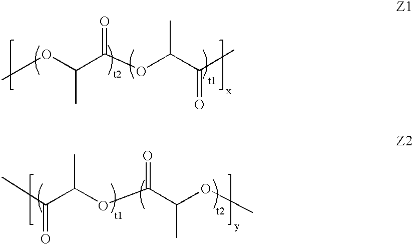
wherein the configuration of the chiral carbons independently for each unit x for Z1 and unit y for Z2 is either D for t1 and L for t2, or L for t1 and D for t2.

44. The composition of claim 43, wherein each of Y1 is O and L1 is —CH(CH₃)CH₂—.

45. The composition of claim 1, wherein said polymer has one or more monomeric units represented by the following Formula VII: 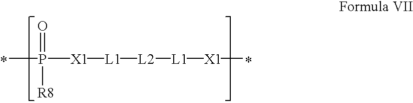
wherein, independently for each occurrence of said monomeric unit:
X1, each independently, represents —O— or —N(R7)-;
R7 represents —H, aryl, alkenyl or alkyl;
L1 represents any chemical moiety that does not materially interfere with the biocompatibility of said polymer;
R8 represents —H, alkyl, —O-alkyl, —O-cycloalkyl, aryl, —O-aryl, heterocycle, —O-heterocycle, or —N(R9)R10;
R9 and R10, each independently, represent a hydrogen, an alkyl, an alkenyl, —(CH₂)m13 R11, or R9 and R10, taken together with the N atom to which they are attached complete a heterocycle having from 4 to about 8 atoms in the ring structure;
m represents an integer in the range of 0-10, preferably 0-6; and
R11 represents —H, alkyl, aryl, cycloalkyl, cycloalkenyl, heterocycle or polycycle; and
L2 represents a divalent, branched or straight chain aliphatic group, a divalent cycloaliphatic group, a phenylene group, or a group of the formula:

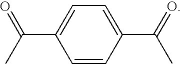

46. The composition of claim 45, wherein each of L1 is —CH₂—.

47. The composition of claim 46, wherein each X1 of each of said units is O.

48. The composition of claim 45, wherein said polymer has one or more monomeric units represented by the following Formula VIII: 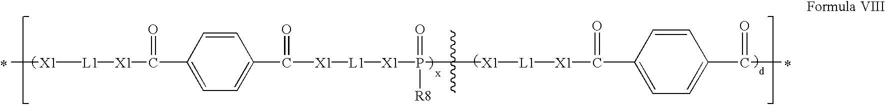
wherein d is equal to one or more and x is equal to or greater than one.

49. The composition of claim 48, wherein each L1 independently represents an alkylene group, a cycloaliphatic group, a phenylene group or a divalent group of the formula: 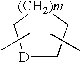
wherein D is O, N or S and m is an integer from 0 to 3.

50. The composition of claim 1, wherein one of said radiosensitizers is a halogenated pyrimidine.

51. The composition of claim 50, wherein said halogenated pyrimidine is IUdR.

52. The composition of claim 1, wherein all of said radiosensitizers are a halogenated pyrimidine.

53. The composition of claim 52, wherein said halogenated pyrimidine is IUdR.

54. A method for treating unwanted cell proliferation of a subject, comprising:
(a) administering to said subject a therapeutically effective amount of one of the compositions claimed above; and (b) treating said subject with electromagnetic radiation.

55. The method of claim 54, wherein said composition is administered by injection into a tumor arising from said unwanted cell proliferation.

56. The method of claim 54, wherein said composition is administered to an anatomic area resulting from removal of a tumor caused by said unwanted cell proliferation.

57. The method of claim 54, wherein said composition is inserted into said patient such that said composition is in at least partial contact with a tumor caused by said unwanted cell proliferation or tissue surrounding the site of said tumor.

58. The method of claim 54, wherein said composition is administered to the central nervous system.

59. The use of a composition in the manufacture of a medicament to treat or prevent a neoplastic growth in a subject, wherein said composition is one of the compositions claimed above.

60. A kit containing a drug delivery system, comprising (a) a composition claimed above, and (b) instructions for administering said composition to a subject with a neoplasm and a treatment of said subject with electromagnetic radiation following said administration.
